(19) United States
(12) Patent Application Publication
(10) Pub. No.: US 2018/0177749 A1
(43) Pub. Date: Jun. 28, 2018

(54) USE OF CYP EXPRESSION TO DIRECT THERAPEUTIC INTERVENTION IN CANCER

(71) Applicant: REGENTS OF THE UNIVERSITY OF MINNESOTA, Minneapolis, MN (US)

(72) Inventors: David Potter, Minneapolis, MN (US); Zhijun Guo, Minneapolis, MN (US)

(21) Appl. No.: 15/831,018

(22) Filed: Dec. 4, 2017

Related U.S. Application Data
(60) Provisional application No. 62/429,647, filed on Dec. 2, 2016.

(51) Int. Cl. A61K 31/155; A61P 35/00; A61K 31/4409; A61K 31/426; A61K 31/417; G01N 33/574

(52) U.S. Cl. CPC A61K 31/155; G01N 2333/90245; A61K 31/4409; A61K 31/426; A61K 31/417; G01N 33/57415; G01N 33/57449; G01N 33/57442; G01N 33/57423; G01N 33/57407; C12Y 303/02009; C12Y 114/14001; G01N 2800/52; G01N 2333/914; A61P 35/00

(57) ABSTRACT

Certain embodiments of the invention provide a method for identifying a cancer cell that is sensitive to a biguanide compound or other CYP epoxygenase inhibitor, comprising detecting increased expression of at least one cytochrome P450 (CYP) and/or decreased expression of soluble epoxide hydrolase (EPHX2) in the cancer cell, wherein increased CYP expression and/or decreased expression of EPHX2 in the cancer cell correlates with increased sensitivity of the cancer cell to the biguanide compound or other CYP epoxygenase inhibitor.

### RELATED APPLICATION

This application claims the benefit of priority of U.S. Provisional Application Ser. No. 62/429,647 filed on Dec. 2, 2016, which application is incorporated by reference herein.

### GOVERNMENT FUNDING

This invention was made with government support under R01-CA113570 awarded by the National Institutes of Health. The government has certain rights in the invention.

### BACKGROUND OF THE INVENTION

Somatic evolution is the accumulation of mutations in the cells of a body during a lifetime, and the effects of those mutations on the fitness of those cells. Somatic evolution is important in the process of aging, as well as the development of some diseases, including cancer. There are multiple levels of genetic heterogeneity associated with cancer, including single nucleotide polymorphism (SNP), sequence mutations, microsatellite shifts and instability, loss of heterozygosity (LOH), and copy number variation and karyotypic variations, including chromosome structural aberrations and aneuploidy. The identification of these mutations and their association with cancer has resulted in a number of clinical benefits, including for determining a patient's prognosis and for identifying patient populations that are likely to benefit from certain drugs.

Thus, there is a need to identify new mutations (e.g., somatic mutations) that are associated with cancer. In particular, there is a need to identify new mutations, which may be used for prognostic indices and to identify patients likely to benefit from certain drugs (e.g., biguanide drugs).

### SUMMARY OF THE INVENTION

Certain embodiments of the invention provide methods for identifying cancer patients likely to benefit from biguanide drugs or other CYP epoxygenase inhibitors. This technology may, for example, streamline clinical trials for new drug development by providing biomarkers that are predictive of clinical benefit. Furthermore, prognostic indices could be developed for certain cancer type, such as breast, ovarian, uterine, bladder cancer, as well as lung adneocarcinoma and glioma. By identifying patients likely to benefit from biguanide drugs, this approach is expected to enable personalized medicine. For example, this technology would allow for the identification of patients who may be more likely to benefit from adjuvant metformin in the NCI MA.32 trial [J Natl Cancer Inst. 2015 Mar. 4; 107(3)].

Accordingly, certain embodiments of the invention provide a method for identifying a cancer cell that is sensitive to a biguanide compound or other cytochrome P450 (CYP) epoxygenase inhibitor, comprising detecting increased expression of at least one CYP and/or decreased expression of soluble epoxide hydrolase (EPHX2) in the cancer cell, wherein increased CYP expression and/or decreased EPHX2 expression in the cancer cell correlates with increased sensitivity of the cancer cell to the biguanide compound or other CYP epoxygenase inhibitor.

Certain embodiments of the invention provide a method for identifying a cancer cell that is sensitive to a biguanide compound or other cytochrome P450 (CYP) epoxygenase inhibitor, comprising: 1) obtaining a nucleic acid or protein sample from the cancer cell; and 2) detecting whether expression of at least one CYP is increased and/or whether expression of soluble epoxide hydrolase (EPHX2) is decreased by measuring the expression level of at least one CYP and/or EPHX2; and 3) identifying the cancer as being sensitive to a biguanide compound or other CYP epoxygenase inhibitor when increased expression of at least one CYP and/or decreased expression of EPHX2 is detected.

Certain embodiments of the invention provide a method for identifying a patient having cancer that can be treated with a biguanide compound or other CYP epoxygenase inhibitor, comprising detecting increased expression of at least one CYP and/or decreased expression of soluble epoxide hydrolase (EPHX2) in a cancer cell sample obtained from the patient, wherein increased CYP expression and/or decreased EPHX2 expression is indicative of a patient with cancer that can be treated with a biguanide compound or other CYP epoxygenase inhibitor.

Certain embodiments of the invention provide a method for identifying a patient having cancer that can be treated with a biguanide compound or other CYP epoxygenase inhibitor, comprising: 1) obtaining a cancer cell sample from the patient; and 2) detecting whether expression of at least one CYP is increased and/or whether expression of soluble epoxide hydrolase (EPHX2) is decreased in a cancer cell from the sample, by measuring the expression level of the at least one CYP and/or EPHX2; and 3) identifying the patient having cancer as being treatable with a biagunide compound or other CYP epoxygenase inhibitor when increased expression of at least one CYP and/or decreased expression of EPHX2 is detected, as compared to a control. In certain embodiments, the method further comprises 4) administering an effective amount of a biguanide compound or other CYP epoxygenase inhibitor to the identified patient.

Certain embodiments of the invention provide a method for identifying a patient having cancer that can be treated with a biguanide compound or other CYP epoxygenase inhibitor, comprising: 1) obtaining a nucleic acid or protein sample from a cancer cell sample obtained from the patient; and 2) detecting whether expression of at least one CYP is increased and/or whether expression of soluble epoxide hydrolase (EPHX2) is decreased by measuring the expression level of the at least one CYP and/or EPHX2; and 3) identifying the patient having cancer as being treatable with a biagunide compound or other CYP epoxygenase inhibitor when increased expression of at least one CYP and/or decreased expression of EPHX2 is detected.

Certain embodiments of the invention provide a method for establishing a prognosis for a patient having cancer, comprising detecting increased expression of at least one cytochrome P450 (CYP) and/or decreased expression of soluble epoxide hydrolase (EPHX2) in a cancer cell sample obtained from the patient, wherein increased CYP expression and/or decreased EPHX2 expression is indicative of a poor prognosis.

Certain embodiments of the invention provide a method for establishing a prognosis for a patient having cancer, comprising: 1) obtaining a cancer cell sample from the patient; and 2) detecting whether expression of at least one CYP is increased and/or whether expression of soluble epoxide hydrolase (EPHX2) is decreased in a cancer cell from the sample, by measuring the expression level of the at least one CYP and/or EPHX2; and 3) establishing the prognosis is poor when increased expression of at least one CYP and/or decreased expression of EPHX2 is detected, as compared to a control. In certain embodiments, the method further comprises 4) administering an effective amount of a biguanide compound or other CYP epoxygenase inhibitor to the patient.

Certain embodiments of the invention provide a method for establishing a prognosis for a patient having cancer, comprising: 1) obtaining a nucleic acid or protein sample from a cancer cell sample obtained from the patient; and 2) detecting whether expression of at least one CYP is increased and/or whether expression of soluble epoxide hydrolase (EPHX2) is decreased by measuring the expression level of the at least one CYP and/or EPHX2; and 3) establishing the prognosis is poor when increased expression of at least one CYP and/or decreased expression of EPHX2 is detected.

Certain embodiments of the invention provide a method for treating a cancer cell comprising administering to the cancer cell an effective amount of a biguanide compound or other CYP epoxygenase inhibitor, wherein the cancer cell was determined to comprise increased expression of at least one CYP and/or decreased expression of soluble epoxide hydrolase (EPHX2).

Certain embodiments of the invention provide a method for treating cancer in a patient comprising administering an effective amount of a biguanide compound or other CYP epoxygenase inhibitor to the patient, wherein the cancer was determined to comprise increased expression of at least one CYP and/or decreased expression of soluble epoxide hydrolase (EPHX2).

Certain embodiments of the invention provide a method comprising 1) detecting increased expression of at least one CYP and/or decreased expression of soluble epoxide hydrolase (EPHX2) in a cancer cell sample obtained from a patient having cancer; 2) identifying the cancer as being sensitive to a biguanide compound or other CYP epoxygenase inhibitor when increased expression of at least one CYP and/or decreased expression of EPHX2 is detected; and 3) administering an effective amount of a biguanide compound or other CYP epoxygenase inhibitor to the patient.

Certain embodiments of the invention provide a method of screening a biguanide compound or other CYP epoxygenase inhibitor for anti-cancer activity, comprising contacting a cancer cell having determined to comprise increased expression of at least one CYP and/or decreased expression of soluble epoxide hydrolase (EPHX2) with a biguanide compound or other CYP epoxygenase inhibitor, wherein sensitivity of the cancer cell to the biguanide compound or other CYP epoxygenase inhibitor is indicative of anti-cancer activity.

Certain embodiments of the invention provide a kit comprising 1) at least one reagent for detecting increased expression of at least one cytochrome P450 (CYP) and/or decreased expression of soluble epoxide hydrolase (EPHX2) in a cancer cell sample; and 2) instructions for (a) using the reagent to detect increased expression of at least one cytochrome P450 (CYP) gene and/or decreased expression of soluble epoxide hydrolase (EPHX2); and (b) to administer a biguanide compound or other CYP epoxygenase inhibitor to a patient having cancer, wherein increased expression of at least one cytochrome P450 (CYP) and/or decreased expression of soluble epoxide hydrolase (EPHX2) is detected in a cancer cell sample from the patient.

Certain embodiments of the invention provide a biguanide compound or other CYP epoxygenase inhibitor for the prophylactic or therapeutic treatment of a cancer determined to comprise increased expression of at least one CYP and/or decreased expression of soluble epoxide hydrolase (EPHX2).

Certain embodiments of the invention provide the use of a biguanide compound or other CYP epoxygenase inhibitor to prepare a medicament for treating cancer in an animal (e.g. a mammal such as a human), wherein the cancer was determined to comprise increased expression of at least one CYP and/or decreased expression of soluble epoxide hydrolase (EPHX2).

### BRIEF DESCRIPTION OF THE FIGURES

FIGS. 1A-G. Cancer cell intrinsic CYP3A4 is required for tumor growth and synthesizes EETs that regulate mitochondrial homeostasis. FIG. 1A. CYP3A4 nanodisc-mediated synthesis of EETs from AA is NADPH-dependent. EETs were extracted and quantified by LC-ESI/MRM/MS. Results are expressed as mean of peak area±S.D. (n=3, * indicates P<0.05). FIG. 1B. Cellular EET regioisomer levels in MCF-7 CYP3A4 knockdown cell lines 3-18 and 4-14 as compared to the NT2 (non-target shRNA) control cell line. EETs were extracted and quantified by LC-ESI/MRM/MS. Results are expressed as mean of EET regioisomer/total protein±S.D. (n=3, * P<0.05). FIG. 1C. Lineweaver-Burk plot for determining the Kₘ of O₂ for CYP3A4 catalyzed epoxidation of arachidonic acid. The rate of O₂ consumption was measured with a continuingly sampling oxygen electrode at varying initial [O₂]: 10, 20, 50, 100, 130, and 220 μM. FIG. 1D. Growth of the NT2 cell line (Δ) or the 3-18 CYP3A4 knockdown cell line (▪) in the mammary fat pad of nude mice (Gompertzian curve fitting; P=0.0101 for difference between the two growth curves). FIG. 1E. Representative H&E-stained images of the 3-18 CYP3A4 knockdown-derived tumors (right) exhibiting central necrosis and control NT2 cell-line derived tumors (left) lacking necrosis (p=0.0152; two tailed Fisher's exact test for presence of necrosis in the knockdown tumors). Size bar 500 μm for both images. FIG. 1F. Co-localization of CYP3A4 and mitochondria, with or without CYP3A4 over-expression. Cells were co-stained with a polyclonal antibody to CYP3A4 (fluorescein secondary antibody) and MitoTracker Red. Arrows indicate peri-nuclear structures that co-stain with MitoTracker-Red and the CYP3A4 antibody. CYP3A4 over-expressing clone C14 and control empty vector clone P7 (Size bar=50 μm). Control images without primary antibody showed no fluorescence (not shown). FIG. 1G. MCF-7 cells treated with the indicated dose of the sEH inhibitor t-AUCB or DMSO vehicle beginning at time point “A” (time of addition) and assayed after addition at 18 minute intervals for OCR (left panel) and ECAR (right panel). OCR was significantly increased at the endpoint by t-AUCB in a concentration dependent fashion (t test at end point of 2.5 μM t-AUCB vs. DMSO vehicle P=0.024; 5 μM P=0.0033).

FIGS. 2A-L. CYP3A4 promotes pAMPK suppression through its EET products, while metformin binds the CYP3A4 active site heme and inhibits EET biosynthesis. FIG. 2A. Western blot of AMPK phosphorylation in MCF-7 CYP3A4 knockdown (shRNA) clones and NT2 (non-target) control line (n=3, P<0.05). FIG. 2B. Western blot of AMPK phosphorylation in the MCF-7 cell line after exposure to (±)-14,15-EET (1 μM) for 24 hours (n=3, P<0.05). Relative protein expression was estimated using GAPDH as an internal standard (A-B). FIG. 2C. CYP3A4 knockdown increases ATP. MCF-7 CYP3A4 knockdown cell lines (3-18 and 4-14) exhibited higher cellular ATP levels than the non-target (NT2) control line. Results are represented as mean±S.D. (n=3, * indicates p<0.05). FIG. 2D. CYP3A4 knockdown increases basal OCR and spare respiratory capacity. The MCF-7 CYP3A4 knockdown cell lines exhibited higher spare respiratory capacity as shown by increased OCR difference between oligomycin inhibition of ATP synthase and FCCP addition, which uncouples the hydrogen potential from ATP synthesis by acting as an H⁺ ionophore. (10⁵ cells per well, n=6) FIG. 2E. CYP3A4 knockdown increases basal ECAR. The MCF-7 CYP3A4 knockdown cell lines exhibited increased ECAR with inhibition of oligomycin and FCCP, suggesting transient catabolic compensation for loss of mitochondrial function. (10⁵ cells per well, n=6) FIG. 2F. Microsomal CYP3A4-mediated biosynthesis of (±)-8,9-, 11,12-, and 14,15-EET after 6 hours exposure to the indicated concentrations of metformin. Data are presented as percent of vehicle treated CYP3A4 microsomal control reactions. FIG. 2G. Cellular EET regioisomers in MCF-7 cells exposed to 5 mM metformin or vehicle for 6 hours. Total EETs were extracted and measured by LC-ESI/MS/MS (*, n=3, P<0.05). EET regioisomer measurements were normalized to cellular protein. FIG. 2H. Type I spin shift induced by metformin interaction with CYP3A4 nanodiscs. Results were derived from a difference spectrum comparing absorbance with and without metformin ligand. FIG. 2I. Determination of the spectral dissociation constant (Kₛ-400 μM) of metformin binding to CYP3A4 nanodiscs. The line is a fitted curve. FIG. 2J. Metformin co-crystalizes with CYP3A4 at the active site near the heme (FIG. 2K). FIG. 2L. Metformin forms a hydrogen bond with R212 in the F-F′ loop of CYP3A4 (stereo view).

FIGS. 3A-G. N1-hexyl-N5-benzyl-biguanide (HBB) potently inhibits MCF-7 cell proliferation, in part, through inhibition of CYP3A4 AA epoxygenase activity. FIG. 3A. Metformin structure. FIG. 3B. HBB structure. FIG. 3C. Type I spin shift induced by HBB interaction with CYP3A4 nanodiscs. (FIG. 3D). Determination of the spectral dissociation constant (Kₛ˜110 μM) of HBB binding to CYP3A4 nanodiscs. The line is a fitted curve. FIG. 3E. Microsomal CYP3A4-mediated biosynthesis of EET regioisomers after 6 hours exposure to the indicated concentrations of HBB. FIG. 3F. Microsomal CYP2C8-mediated biosynthesis of EETs after 6 hours exposure to the indicated concentrations of HBB. FIG. 3G. MCF-7 cell growth after treatment with metformin, buformin, phenformin, or HBB at the indicated concentrations in the presence or absence of concurrent treatment with (±)-14,15-EET (1 μM). Cell growth was measured by MTT assay at 24 hours after addition of biguanide or vehicle (PBS vehicle for metformin, DMSO for the other biguanides) and (±)-14,15-EET (1 μM) or vehicle (ethanol). Results are expressed as percent growth relative to control, mean±S.E.M, n=8. (* P<0.05)

FIGS. 4A-E. HBB inhibits OCR and ECAR and suppresses the ETC in part through reduction of (±)-14,15-EET. Basal oxygen consumption rate (OCR) and extra cellular acidification rate (ECAR) of MCF-7 cells were measured at 9-minute interval for 45 minutes (10⁵ cells per well). At time A, treatment was added and measurement continued for an additional 270 minutes at 18-minute intervals. FIG. 4A. OCR and ECAR after treatment with metformin (2.5 and 5 mM) or PBS control were not significantly different (by Student t test) after 270 minutes of treatment. Total cellular ATP levels were unchanged after 6 hours of metformin treatment (right panel). FIG. 4B. OCR and ECAR after treatment with HBB (20 and 40 μM) vs. DMSO. OCR was reduced by HBB vs. DMSO and was significant at the endpoint of A+270 minutes (by t test, HBB 20 μM vs. DMSO P=0.0014; HBB 40 μM vs. DMSO P=2.5×10⁻⁵). ECAR was reduced after treatment with HBB vs. DMSO and was significant at the endpoint of A+270 minutes (P=HBB 20 μM vs. DMSO P=0.0021 and HBB 40 μM vs. DMSO P=0.00050, by t test). Total cellular ATP levels were reduced at 6 hours of HBB treatment (P<0.05 by t test; right panel). FIG. 4C. Pre-treatment with soluble epoxide hydrolase inhibitor t-AUCB (5 μM) vs. DMSO control partially protects OCR but not ECAR from HBB-mediated inhibition. t-AUCB or DMSO was added before addition of HBB (20 μM) at time A (A−120 minutes). OCR and ECAR were measured at 18-minute intervals until the A+270 minute endpoint where OCR was higher in the t-AUCB (5 μM) pre-treatment condition (P=0.00011, by t test), but ECAR was not (P=0.33, by t test). All results are presented as mean±S.D (n=5). FIG. 4D. ΔΨm, visualized by JC-1 dye staining, is reduced by HBB in MCF-7 cells and partially restored by EET. Representative images are shown. Compared to control (left upper panel), HBB (20 μM) treatment for 4 hours caused mitochondrial depolarization in MCF-7 cells (right upper panel) and (±)-14,15-EET (1 μM) provided partial protection (right bottom panel); (±)-14,15-EET (1 μM) alone increased mitochondrial cross membrane potential (left bottom panel) relative to control. Size bar 200 μm. FIG. 4E. Red/green ratio in experiments shown in FIG. 4D. was quantified and presented as results of mean±S.D (n=4) (* indicates P<0.05).

FIGS. 5A-C. HBB treatment results in early and progressive AMPK activation, transient early S6 kinase activation and later mTOR and ERK inhibition in the presence or absence of serum. FIG. 5A. Western blot of signaling proteins of MCF-7 cells grown in complete media (10% serum) or serum starved (˜16 hours) and treated with HBB (20 μM). FIGS. 4B-C. Quantification of the western blotting analysis in FIG. 5A (n=3, * indicates p<0.05).

FIGS. 6A-D. HBB inhibits the estradiol-dependent MCF-7 xenograft with reversible weight loss. FIG. 6A. MCF-7 mammary fat pad xenograft model treated with HBB vs. vehicle. Arrow indicates the time of treatment initiation when tumors averaged 50 mm³ (n=15 mice per group). Daily treatment (6 mg/kg ip) was given for the first three days, followed by weight loss. The treatment schedule was then changed to 4 days of 7 with no more than 2 consecutive days of dosing, repeating each week. Tumor size in the HBB group was smaller at the endpoint of treatment (*; P=0.039). FIG. 6B. Weights of mice treated with either vehicle or HBB. Arrow indicates the time of treatment initiation. Maximum weight differential of 11% on day 16 recovered to 6% by day 38. FIG. 6C. Tumor weights were lower in the HBB treated mice at the endpoint of the xenograft study (A.) (*; P=0.034). FIG. 6D. Model for HBB Inhibition of CYP3A4 Epoxygenase Activity. Upper panel: HBB inhibits CYP3A4 at the plasma or endoplasmic reticulum membrane, thereby activating AMPK by removing EET-mediated inhibition. The mechanism by which EET suppresses AMPK is unknown. HBB also inhibits Stat3 and mTOR by suppressing EET, which is required for activation. Lower panel: HBB inhibits CYP3A4 at the mitochondrion, resulting in immediate loss of OCR and sustained loss of ECAR, in part, due to depletion of ATP. Under conditions of sustained HBB exposure, crosstalk occurs between these two sites of CYP3A4 function. While activation of AMPK phosphorylation supports catabolism, ECAR is suppressed, in part, due to depletion of OCR-dependent ATP production, which is needed for priming of glycolysis.

FIGS. 7A-B. CYP3A4 Correlates with ERα in Breast Cancer. FIG. 7A. CYP3A4 and ERα staining in representative tumor cores of breast cancer from patients sequentially enrolled in a tumor registry. CYP3A4 staining is seen in both epithelia and stroma and images are arrayed across a range of staining, from upper left to lower right. Size bar is 50 μm; estimated from the 750×750 μm field of view of the TMA image. FIG. 7B. Correlation of cytoplasmic CYP3A4 and nuclear ERα expression in the epithelia of unselected breast tumors (Pearson's correlation coefficient=0.7575; P<0.0001). CYP3A4 staining was defined within an epithelial mask derived using co-staining cytokeratin 8 (CK8) antibody, while the field of ERα measurement was defined by a nuclear mask derived from DAPI staining and each field was quantified by the AQUA method of immunofluorescence quantification.

FIGS. 8A-B. EETs support Mitochondrial Respiration in MDA-MB-231 cells. FIG. 8A. MDA-MB-231 cells treated with t-AUCB (2.5 or 5 μM) or DMSO vehicle beginning at time point (A) and assayed at 18-minute intervals for OCR and ECAR. OCR was significantly increased at the endpoint in a concentration dependent fashion (t test at endpoint of 2.5 μM t-AUCB vs. DMSO vehicle P=0.023; 5 μM t-AUCB vs. DMSO vehicle P=0.00080). FIG. 8B. Addition of (±)-14,15-EET (1.0 μM) to the MDA-MB-231 cell line had no effect on AMPK phosphorylation (24 hours).

FIGS. 9A-B. Metformin Inhibits Clonogenicity of the MCF-7 Cell Line, Which is Restored by EET. FIG. 9A. Clonogenicity of MCF-7 cells (n=200, estimated by cell counting and serial dilution) after treatment overnight (24 hours) with ethanol vehicle (control), metformin (1 mM) and vehicle or metformin (1 mM) and (±)-14,15-EET (1 μM). After 24 hours of culture, the medium was then removed followed by addition of CM to the plates (left panel). The plates were then cultured for 2 weeks and at the endpoint the cell colonies were stained with crystal violet and the colonies counted. Results represent mean±standard deviation (n=3, *, ** indicate statistically significant difference with other two groups, P<0.05). FIG. 9B. Metformin (1 mM) treatment for 48 hours did not change CYP3A4 protein levels in the MCF-7 cell line (right panel).

FIGS. 10A-D. Metformin Binds Soluble CYP3A4 with High Affinity While Leaving CPR Function Unaffected and the co-Crystal Serves as a Model for HBB Docking. FIG. 10A. Metformin binding to CYP3A4Δ3-22 is saturable and leads to a weak type II spin shift in the Soret band as evidenced from the difference spectrum indicating the spin shift (left inset). The Kₛ spectral binding constant was ˜2 μM (right inset). The spectra are ligand free CYP3A4Δ3-22, metformin bound while CO indicates carbon monoxide inactivation and DT dithionite reduction of the heme. FIG. 10B. Metformin failed to inhibit cytochrome P450 reductase (CPR)-mediated reduction of cytochrome c (1 and 10 mM metformin). Reaction was initiated by the addition of CPR (final concentration 0.2 mg/mL) to 1 mL potassium phosphate buffer (0.3 M, pH=8.3) containing cytochrome c (62 mM) and NADPH 50 (mM) in the presence or absence of metformin. Absorbance at 550 nm was monitored in real time. Results are shown with one representative curve of each condition, consistent with replicate data. FIG. 10C. HBB (left) docks in the CYP3A4-metformin co-crystal structure (right) (FIG. 10D).

FIG. 11. HBB Binds Soluble CYP3A4. HBB docked to truncated CYP3A4Δ3-22 induces a pronounced type I spin shift with a Kₛ=164 μM. The HBB binding spectrum is shown (HBB-bound). Control spectra with dithionite reduction (ferrous) and CO adduct formation (CO adduct) are shown as well as untreated CYP3A4Δ3-22 (ferric).

FIGS. 12A-B. EETs Rescue the T47D Cell Line from HBB, but not MDA-MB-231. FIG. 12A. The presence of (±)-14,15-EET (1 μM) partially protected ER+T47D, but not triple negative MDA-MB-231 (FIG. 12B) cells against HBB-mediated inhibition of proliferation at 24 hours. Results are expressed as percent growth relative to vehicle control (mean±S.E.M, n=8) and were significant from 10 to 50 μM HBB for the T47D cell line (P<0.05), but not the MDA-MB-231 cell line.

FIGS. 13A-D. HBB Inhibits OCR and ECAR in ER+ and Triple Negative Breast Cancer Cell Lines and Inhibits ΔΨm. HBB effects on basal oxygen consumption rate (OCR) and extra cellular acidification rates (ECAR) of ER+ and triple negative breast cancer cell lines, similar to a rotenone control experiment. At time “A”, treatment was added and measurements were conducted at 18-minute intervals for 270 minutes. FIG. 13A. Effect of 1 μM rotenone on MCF-7 cells. Reduction of OCR and ECAR was highly significant (P<0.05). FIGS. 13B-D. Effect of HBB (20 and 40 μM) on T47D, MDA-MB-231, and MDA-MB-435/LCC6. All data points are presented as mean±S.D. (n=5). The reduction of OCR and ECAR by HBB was significant vs. vehicle and by endpoint analysis was significant for rotenone treatment of the MCF-7 cell line (P<0.01 for both). Reduction of OCR and ECAR by HBB (20 and 40 μM) was significant vs. vehicle for the T47D, MDA-MB-231, and MDA-MB-435/LCC6 cell lines by curve fitting; significant for all comparisons of DMSO vs. 20 and 40 μM and 20 vs. 40 μM HBB (OCR and ECAR endpoints; HBB vs. DMSO control P all <0.015).

FIGS. 14A-B. HBB Inhibits ΔΨm of the MDA-MB-231 Cell Line, Partially Reversed by Exogenous (±)-14,15-EET. FIG. 14A. ΔΨm, visualized by JC-1 dye staining, was inhibited by HBB in MDA-MB-231 cells and partially restored by (±)-14,15-EET. Representative images are shown. Compared to control (left upper panel), HBB (20 μM) treatment for 4 hours caused mitochondrial depolarization in MDA-MB-231 cells (right upper panel) and (±)-14,15-EET (1 μM) provided partial protection (right bottom panel); (±)-14,15-EET (1 μM) alone increased mitochondrial membrane potential (left bottom panel) relative to control. Size bar 200 μm. FIG. 14B. Red/green ratio in experiments shown in FIG. 14A was quantified and presented as results of mean±S.D (n=4) (* indicates P<0.05).

FIGS. 15A-D. Identification of the Minimal Effective Dose (MED) for HBB in the MCF-7 Tumors. MCF-7 or MDA-MB-231 orthotopic mammary fat pad xenograft models treated with HBB or vehicle with daily dosing at the minimum effective dose (MED) beginning the day after tumor cell implantation. FIG. 15A. MCF-7 mammary fat pad xenograft model treated with HBB or vehicle. Arrow indicates the time of treatment initiation with HBB at 4 mg/kg/day or PBS (n=20 mice per group). Tumor size in the HBB group was smaller at indicated times (*), and by Gompertizan fitting the tumors of the HBB treated group were smaller than the vehicle-treated group from day 8 onward (P=0.0354). FIG. 15B. MDA-MB-231 mammary fat pad xenograft model treated with HBB or vehicle. Arrow indicates the time of treatment initiation with HBB at 4 mg/kg/day or PBS (n=20 mice per group). Tumor size in the HBB-treated group was indistinguishable from the vehicle-treated group. FIG. 15C. Weights of mice bearing MCF-7 tumors treated with either HBB or PBS. Arrow indicates the time of treatment initiation. FIG. 15D. Weights of mice bearing MDA-MB-231 tumors treated with either HBB or PBS. Arrow indicates the time of treatment initiation.

FIGS. 16A-C. CYP2J2 gene expression in the METABRIC breast cancer cohort (n=1980) is associated with decreased OS and DFS (FIGS. 16A,B) (P=8.259e-4 and P=2.984e-4; n=39). CYP2J2 gene expression was most strongly associated with HER2 and basal/normal subtypes (FIG. 16C).

FIG. 17. CYP4A11 gene expression in the METABRIC breast cancer cohort (n=1980) is associated with decreased OS and exhibits a trend towards late DFS (FIGS. 17A,B) (P=0.0229 and P=0.401; n=49). CYP4A11 gene expression was most strongly associated with luminal A and B subtypes (FIG. 17C).

FIGS. 18A-B. FIG. 18A. CYP2J2 and CYP4A11 up-regulation or down-regulation by PAM50 subtype in the METABRIC breast cancer cohort (n=1980). Up-regulation of these genes trends with different breast cancer subtypes. Gene expression is altered in 10% of breast cancer. Legend: basal=1, 2=HER2, 3=luminal A, 4=luminal B, 5=normal. FIG. 18B. CYP2J2 and CYP4A11 up-regulation or down-regulation is nearly mutually exclusive.

FIGS. 19A-C. Combined CYP2J2 and 4A11 gene expression in the METABRIC breast cancer cohort (n=1980) is associated with decreased OS and DFS (FIGS. 19A,B) (P=0.000132 and P=0.00460; n=89). CYP4A11 gene expression was most strongly associated with luminal A and B subtypes (FIG. 19C).

FIGS. 20A-B. CYP4A11 gene expression in the TGCA-provisional breast cancer cohort (n=1098) is associated with decreased OS but not DFS (FIGS. 20A,B) (P=0.00357 for OS; n=35).

FIG. 21. CYP3A11 bactosome mediated EET biosynthesis (top left panel). CYP4F2B bactosome mediated EET biosynthesis (tope right panel). Effect of metformin on CYP2J2-mediated EET biosynthesis (middle left panel). Effect of metformin on CYP4F3B-mediated EET biosynthesis (middle right panel). Effect of HBB on CYP4A11-mediated EET biosynthesis (bottom left panel). Effect of HBB on CYP2J2-mediated EET biosynthesis (bottom right panel).

FIGS. 22A-D. CYP4A11 expression correlates with poorer overall survival. FIG. 22A. CYP3A4 and CYP4A11 copy number variation for breast invasive carcinoma (TGCA database). Correlation between CYP4A11 expression and overall survival (OS) (FIG. 22B) and disease free survival (DFS) (FIG. 22C); data derived from TGCA database. Correlation between CYP4A11 expression and overall survival (OS) (FIG. 22D); data derived from the METABRIC database.

FIGS. 23A-C. Analysis of CYP4A11 using TGCA provisional database.

FIGS. 24A-E. Analysis of CYP4A11 using METABRIC database.

### DETAILED DESCRIPTION

Although metformin and other biguanide drugs have been explored as novel anti-cancer agents and inhibit mitochondrial function, the targets of biguanide drugs in cancer have been unknown. A major gap in knowledge has been a lack of identification of a cognate target to which metformin binds and inhibits function. As described herein, it has been recently discovered using X-ray crystallography and CYP nanodiscs that cytochrome P450 enzymes are targets of metformin and other biguanides. Furthermore, co-localization of CYP3A4 and mitochondria has been established. These results establish the first direct evidence for a mitochondrial-associated metformin target in patients. Furthermore, it has been discovered that biguanides suppress cancer growth, in part, by inhibiting CYP-mediated biosynthesis of cancer promoting eicosanoids, such as epoxyeicosatrienoic acids (EETs). EETs inhibit the tumor suppressor AMPK, drive mitochondrial respiration and promote the Warburg effect by which cancer cells divert carbon units to build biomass, thereby contributing to cancer progression. Because gene amplification is an important mechanism of oncogenesis, it was asked whether CYP genes are amplified in solid tumors and discovered this process to be widespread in cancer and correlates with poor outcomes using the Cancer Genome Atlas (TGCA). Specifically, in the case of breast cancer, it was found that CYP(s) are amplified in 1.7% of patients (METABRIC), but in other cancer types the prevalence of CYP amplification is higher: 11.4% of ovarian adenocarcinoma (TGCA), 13.6% of uterine carcinoma (TGCA), 14% of bladder cancer (TGCA) 15.7% of lung adenocarcinoma (TGCA) and 7.5% of low grade glioma (TGCA). Accordingly, certain embodiments of the invention provide methods for identifying tumors that are dependent on CYP gene amplification for their growth and which are vulnerable to inhibition with biguanide compounds (e.g., metformin and other more potent biguanide drugs, such as hexyl-benzyl-biguanide (HBB)). Specifically, it was discovered that CYP gene amplification patterns correlate with worse prognosis in many solid tumor types, including breast, ovarian, endometrial and bladder cancer, low grade glioma and lung adenocarcinoma. Review of large patient cohorts (>500 subjects) from the TGCA (breast, ovarian, uterine, lung, bladder cancer and glioma) and METABRIC (breast cancer) studies indicates that CYP gene amplification and/or deletion of soluble epoxide hydrolase (EPHX2) correlates with disease free survival and/or overall survival in these large patients cohorts (more than 500 patients per study) including TGCA. Furthermore, it was found that amplification of CYP3A4/5 and CYP4F2/3 with or without CYP2J2 may be sufficient to identify poor prognosis breast, ovarian, uterine, bladder cancer, as well as lung adenocarcinoma and glioma. Therefore, it is hypothesized that CYP monooxygenase gene amplification is a common mechanism of cancer progression in solid tumors through increased CYP biosynthesis of cancer-promoting eicosanoids, including epoxyeicosatrienoic acids (EETs), and can be used through recognition of amplification fingerprints to identify tumors sensitive to metformin and HBB through, e.g., PCR or FISH technology. Deletion of soluble epoxide hydrolase (EPHX2), which hydrolyzes EETs, also correlates with CYP gene amplification in certain tumor types and can contribute to some models. It is therefore proposed that copy number variation of CYP and/or EPHX2 be measured using, e.g., PCR and/or FISH, which may be prognostic for outcomes in breast, ovarian, uterine, bladder cancer, lung adenocarcinoma and glioma. Furthermore, it is proposed that amplification of CYP genes and/or EPHX2 deletion is predictive of patients who would likely to benefit from biguanide compounds, such as metformin, HBB and novel neobiguanide drugs. The methods described herein also have a potential prognostic and predictive model that can inform clinical development of biguanide drugs for cancer.

### Methods of the Invention

Certain embodiments of the invention provide a method for identifying a cancer cell that is sensitive to a biguanide compound or other CYP epoxygenase inhibitor, comprising detecting increased expression of at least one cytochrome P450 (CYP) and/or decreased expression of soluble epoxide hydrolase (EPHX2) in the cancer cell, wherein increased expression of at least one CYP and/or decreased expression of EPHX2 in the cancer cell correlates with increased sensitivity of the cancer cell to the biguanide compound or other CYP epoxygenase inhibitor. In certain embodiments, the method is for identifying a cancer cell that is sensitive to a biguanide compound.

Certain embodiments of the invention provide a method for identifying a cancer cell that is sensitive to a biguanide compound or other CYP epoxygenase inhibitor, comprising: 1) providing a nucleic acid or protein sample obtained from the cancer cell; and 2) detecting increased expression of at least one CYP and/or decreased expression of soluble epoxide hydrolase (EPHX2) in the sample; wherein increased CYP expression and/or decreased expression of EPHX2 in the cancer cell correlates with increased sensitivity of the cancer cell to the biguanide compound or other CYP epoxygenase inhibitor.

Certain embodiments of the invention provide a method for identifying a cancer cell that is sensitive to a biguanide compound or other CYP epoxygenase inhibitor, comprising: 1) obtaining a nucleic acid or protein sample from the cancer cell; and 2) detecting whether expression of at least one CYP is increased and/or whether expression of soluble epoxide hydrolase (EPHX2) is decreased by measuring the expression levels of at least one CYP and/or EPHX2; and 3) identifying the cancer as being sensitive to a biguanide compound or other CYP epoxygenase inhibitor when increased expression of at least one CYP and/or decreased expression of EPHX2 is detected.

Certain embodiments of the invention provide a method for identifying a patient having cancer that can be treated with a biguanide compound or other CYP epoxygenase inhibitor, comprising detecting increased expression of at least one cytochrome P450 (CYP) and/or decreased expression of soluble epoxide hydrolase (EPHX2) in a cancer cell sample obtained from the patient, wherein increased CYP expression and/or decreased expression of EPHX2 is indicative of a patient with cancer that can be treated with a biguanide compound or other CYP epoxygenase inhibitor. In certain embodiments, the method is for identifying a patient having a cancer than can be treated with a biguanide compound.

Certain embodiments of the invention provide a method for identifying a patient having cancer that can be treated with a biguanide compound or other CYP epoxygenase inhibitor, comprising: 1) providing a nucleic acid or protein sample derived from a cancer cell sample obtained from the patient; and 2) detecting increased expression of at least one CYP and/or decreased expression of soluble epoxide hydrolase (EPHX2) in the sample; wherein increased CYP expression and/or decreased expression of EPHX2 is indicative of a patient with cancer that can be treated with a biguanide compound or other CYP epoxygenase inhibitor.

Certain embodiments of the invention provide a method for identifying a patient having cancer that can be treated with a biguanide compound or other CYP epoxygenase inhibitor, comprising: 1) obtaining a nucleic acid or protein sample from a cancer cell sample obtained from the patient; and 2) detecting whether expression of at least one CYP is increased and/or whether expression of soluble epoxide hydrolase (EPHX2) is decreased by measuring the expression levels of at least one CYP and/or EPHX2; and 3) identifying the patient having cancer as being treatable with a biguanide compound or other CYP epoxygenase inhibitor when increased expression of at least one CYP and/or decreased expression of EPHX2 is detected.

Certain embodiments of the invention provide a method for establishing a prognosis for a patient having cancer, comprising detecting increased expression of at least one cytochrome P450 (CYP) and/or decreased expression of soluble epoxide hydrolase (EPHX2) in a cancer cell sample obtained from the patient, wherein increased CYP expression and/or decreased expression of EPHX2 is indicative of a poor prognosis (e.g., as compared to a patient having a corresponding cancer, wherein increased expression of at least one CYP and/or decreased expression of EPHX2 is not detected).

Certain embodiments of the invention provide a method for establishing a prognosis for a patient having cancer, comprising: 1) providing a nucleic acid or protein sample derived from a cancer cell sample obtained from the patient; and 2) detecting increased expression of at least one CYP and/or decreased expression of soluble epoxide hydrolase (EPHX2) in the nucleic acid or protein sample; wherein increased CYP expression and/or decreased expression of EPHX2 is indicative of a poor prognosis (e.g., as compared to a patient having a corresponding cancer, wherein increased expression of at least one CYP gene and/or decreased expression of EPHX2 is not detected).

Certain embodiments of the invention provide a method for establishing a prognosis for a patient having cancer, comprising: 1) obtaining a nucleic acid or protein sample from a cancer cell sample obtained from the patient; and 2) detecting whether expression of at least one CYP is increased and/or whether expression of soluble epoxide hydrolase (EPHX2) is decreased by measuring the expression levels of the at least one CYP and/or EPHX2; and 3) establishing the prognosis is poor when increased expression of at least one CYP gene and/or decreased expression of EPHX2 is detected (e.g., as compared to a patient having a corresponding cancer, wherein increased expression of at least one CYP and/or decreased expression of EPHX2 is not detected).

Certain embodiments of the invention provide a method for treating a cancer cell comprising administering to the cancer cell an effective amount of a biguanide compound or other CYP epoxygenase inhibitor, wherein the cancer cell was determined to comprise increased expression of at least one CYP and/or decreased expression of soluble epoxide hydrolase (EPHX2). In certain embodiments, a biguanide compound is administered.

Certain embodiments of the invention provide a method for treating cancer in a patient comprising administering an effective amount of a biguanide compound or other CYP epoxygenase inhibitor to the patient, wherein the cancer was determined to comprise increased expression of at least one CYP and/or decreased expression of soluble epoxide hydrolase (EPHX2). In certain embodiments, a biguanide compound is administered.

Certain embodiments of the invention provide a biguanide compound or other CYP epoxygenase inhibitor for the prophylactic or therapeutic treatment of a cancer determined to comprise increased expression of at least one CYP and/or decreased expression of soluble epoxide hydrolase (EPHX2).

Certain embodiments of the invention provide the use of a biguanide compound or other CYP epoxygenase inhibitor to prepare a medicament for treating a cancer in an animal (e.g. a mammal such as a human) determined to comprise increased expression of at least one CYP and/or decreased expression of soluble epoxide hydrolase (EPHX2).

Certain embodiments of the invention provide a method comprising 1) detecting increased expression of at least one CYP gene and/or decreased expression of soluble epoxide hydrolase (EPHX2) in a cancer cell sample obtained from a patient having cancer; 2) identifying the cancer as being sensitive to a biguanide compound or other CYP epoxygenase inhibitor when increased expression of at least one CYP gene and/or decreased expression of EPHX2 is detected; and 3) administering an effective amount of a biguanide compound or other CYP epoxygenase inhibitor to the patient. In certain embodiments, a biguanide compound is administered.

In certain embodiments, the methods further comprise administering a second therapeutic agent. In certain embodiments, the second therapeutic agent is useful for treating cancer (e.g., a chemotherapeutic agent, hormonal agents or radiation therapy). In certain embodiments, the second therapeutic agent inhibits at least one CYP. In certain embodiments, the second therapeutic agent is a pan CYP inhibitor. In certain embodiments, the second therapeutic agent is a selective CYP inhibitor. In certain embodiments, the second therapeutic agent is selected based on the CYP expression data (an inhibitor of the particular CYP(s) that have increased expression may be administered). In certain embodiments, the second therapeutic agent inhibits EET biosynthesis. For example, if expression of CYP2J2 is increased, then micardis (telmisartan) may be administered (e.g., HBB may be administered in combination with micardis (telmisartan) if CYP3A4 and CYP2J2 have increased expression; e.g., in HER2+ and triple negative breast cancer ER−, PR−, HER2−); or if expression of CYP4A11 is increased, then sesamin (5,5′-(1S,3aR,4S,6aR)-tetrahydro-1H,3H-furo[3,4-c]furan-1,4-diylbis(1,3-benzodioxole)), HET0016, N-hydroxy-N′-(4-n-butyl-2-methylphenyl)formamidine, DDMS or dibromo-dodecenyl-methylsulfimide may be administered (e.g., HBB may be administered in combination with sesamin if CYP3A4 and CYP4A11 have increased expression; e.g., in ER+ breast cancer) (Ren et al., Drug Metabolism and Disposition, 2013; 41:60-71; Wu et al., Hypertension 2009; 54:1151-1158, which are incorporated by reference herein). DDMS, dibromo-dodecenyl-methylsulfimide, HET0016 or N-hydroxy-N′-(4-n-butyl-2-methylphenyl)formamidine may be used as an inhibitor of CYP4A11 omega hydroxylase activity to block synthesis of 20 HETE and may inhibit CYP4A11 biosynthesis of epoxyeicosatrienoic acids. Both of these eicosanoids are known to promote tumor progression.

In certain embodiments, the second therapeutic agent is a chemotherapeutic agent. In certain embodiments, the chemotherapeutic agent is selected from tamoxifen, fulvestrant, raloxifene, anastrozole, letrozole, exemestane, paclitaxel, docetaxel, ixabepilone, eribulin, capecitabine, gemcitabine, vinorelbine, palbociclib, everolimus, trastuzumab, pertuzumab, lapatinib and other HER2 receptor tyrosine kinase inhibitors. In certain embodiments, the second therapeutic agent is paclitaxel. In certain embodiments, a combination of HBB and paclitaxel are administered. In certain embodiments, the second agent is activates the immune system. In certain embodiments the second agent is an immune checkpoint inhibitor antibody such as anti PD-1 or anti PD-L1. In certain embodiments, the second agent is an immune activator, such as HBB.

The second therapeutic agent may be administered either simultaneously or sequentially with the biguanide compound or other CYP epoxygenase inhibitor. In certain embodiments, the second therapeutic agent is administered simultaneously with the biguanide compound or other CYP epoxygenase inhibitor. In certain embodiments, a composition (e.g., a pharmaceutical composition) comprising the biguanide compound or other CYP epoxygenase inhibitor and the second therapeutic agent is administered. In certain embodiments, the biguanide compound or other CYP epoxygenase inhibitor and the second therapeutic agent are administered sequentially. In certain embodiments, the biguanide compound or other CYP epoxygenase inhibitor is administered first and the second therapeutic agent is administered second. In certain embodiments, the second therapeutic agent is administered first and biguanide compound or other CYP epoxygenase inhibitor is administered second.

Certain embodiments of the invention provide a method for detecting the presence of a biomarker in a cancer cell, the improvement comprising detecting increased expression of at least one CYP gene and/or decreased expression of soluble epoxide hydrolase (EPHX2) in the cancer cell for use in predicting sensitivity of the cancer cell to a biguanide compound or other CYP epoxygenase inhibitor, wherein increased CYP expression and/or decreased expression of EPHX2 in the cancer cell correlates with increased sensitivity of the cancer cell to the biguanide compound.

Certain embodiments of the invention provide a method of screening a biguanide compound or other CYP epoxygenase inhibitor for anti-cancer activity, comprising contacting a cancer cell comprising increased expression of at least one CYP and/or decreased expression of soluble epoxide hydrolase (EPHX2) with a biguanide compound or other CYP epoxygenase inhibitor, wherein sensitivity of the cancer cell to the biguanide compound or other CYP epoxygenase inhibitor is indicative of anti-cancer activity.

Certain embodiments of the invention provide a method of screening a biguanide compound or other CYP epoxygenase inhibitor for anti-cancer activity, comprising 1) contacting a cancer cell comprising increased expression of at least one CYP and/or decreased expression of soluble epoxide hydrolase (EPHX2) with a biguanide compound or other CYP epoxygenase inhibitor; and 2) measuring hydroxyeicosatetraenoic acid (HETE) and/or epoxyeicosatrienoic acid (EET) biosynthesis; wherein a decrease in HETE and/or EET biosynthesis is indicative of a biguanide compound or other CYP epoxygenase inhibitor having anti-cancer activity (e.g., as compared to a control, such as HETE and/or EET biosynthesis in the absence of the compound/inhibitor).

For example, in certain embodiments, the CYP is CYP3A4 and the biguanide compound is HBB (e.g., breast cancer). In such an assay, decreased EET biosynthesis may be observed, indicating that HBB would be an effective anti-cancer agent and could be administered.

In certain embodiments, the HETE and/or EET biosynthesis is measured using mass spectrometry. In certain embodiments, the HETE and/or EET biosynthesis is measured in the presence of arachidonic acid (AA).

In certain embodiments EET biosynthesis is measured. In certain embodiments, an EET described herein is measured. In certain embodiments, 14,15-EET is measured. In certain embodiments, 11,12-EET is measured.

In certain embodiments, HETE biosynthesis is measured. In certain embodiments, 20-HETE is measured.

As used herein, the term “sensitive to a biguanide compound or other CYP epoxygenase inhibitor” and “sensitivity of a cancer cell to a biguanide compound or other CYP epoxygenase inhibitor” refers to a cancer cell that has decreased growth, proliferation and/or dies when contacted with a biguanide compound or other CYP epoxygenase inhibitor (e.g., a biguanide compound is administered to a patient).

As used herein, the term “increased expression” refers to an increase in mRNA or protein expression levels. For example, the increase in expression may result from a mutation, gene amplification (i.e., an increase in gene copy number), increased transcription, increased translation or decreased degradation at the mRNA or protein level. To establish whether expression is increased, expression levels may be compared to a control. For example, comparison may be made to the expression level of a corresponding CYP from a corresponding non-cancerous cell. Additionally, as described herein, expression may also be normalized using an internal control in certain embodiments.

As used herein, the term “gene amplification” refers to an increase in the number of copies of a gene (e.g., as compared to the number of copies of the gene in a control cell, such as a non-cancerous cell). In certain embodiments, gene amplification results in an increase in the RNA and/or protein made from that gene.

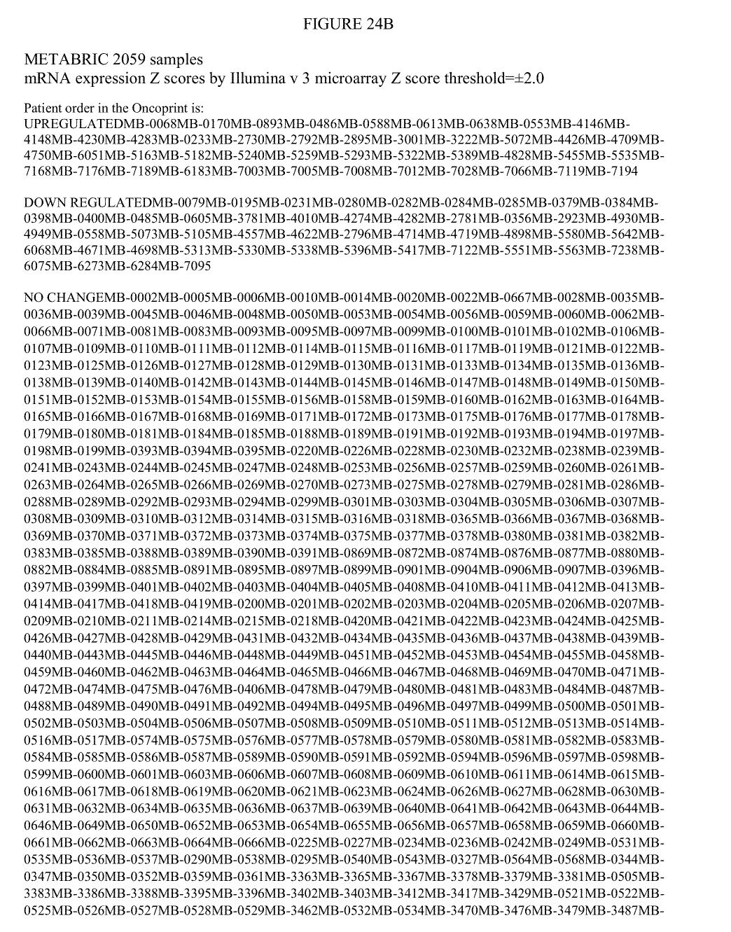

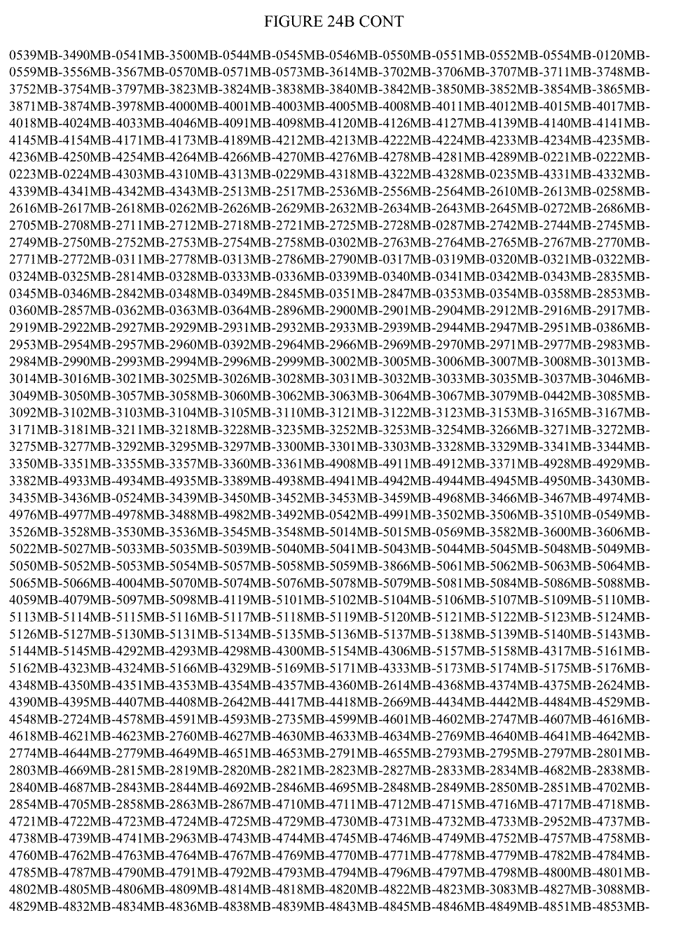

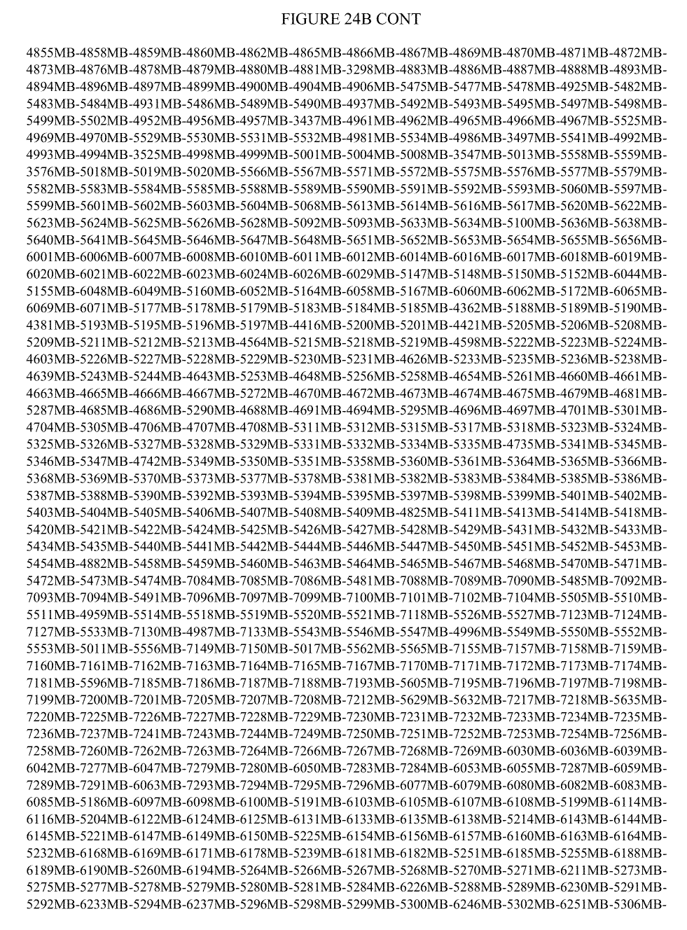

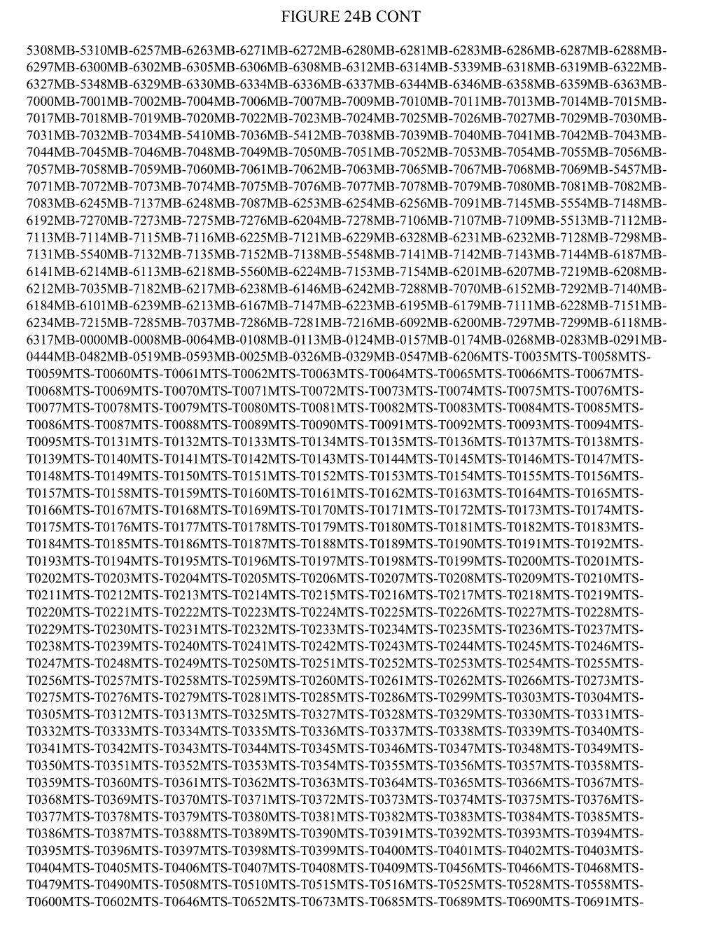

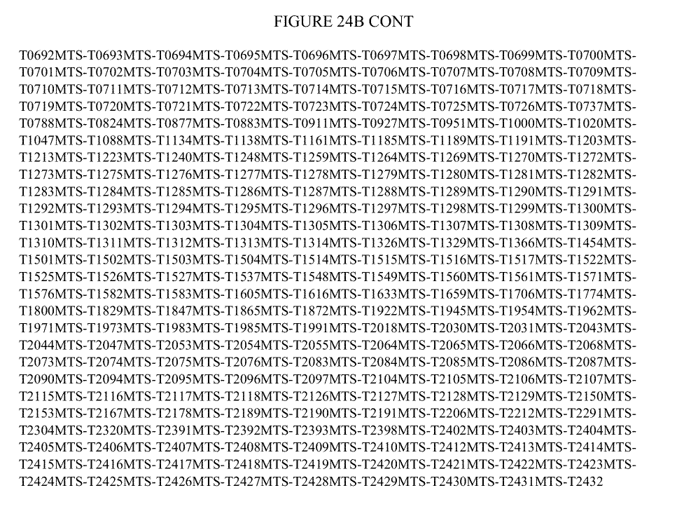

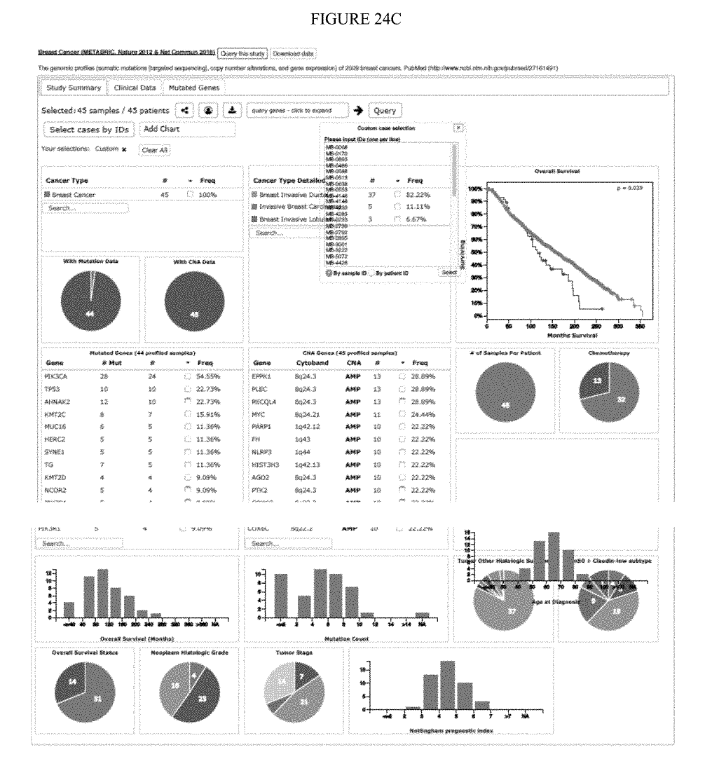

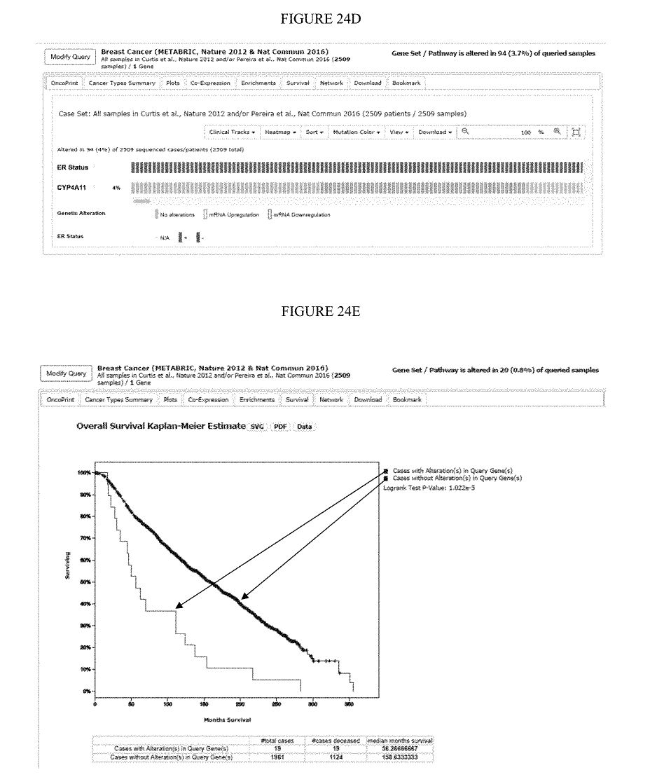

Accordingly, in certain embodiments, increased mRNA expression is detected (e.g., increased mRNA expression is detected for at least one CYP). For example, in certain embodiments, increased mRNA expression of CYP4A11 is detected (e.g., in breast cancer). In certain embodiments, increased protein expression is detected (e.g., increased mRNA expression is detected for at least one CYP). For example, in certain embodiments increased protein expression of CYP3A4 is detected (e.g., in breast cancer). In certain embodiments, amplification of at least one CYP gene is detected. For example CYP4A11 amplification correlates with decreased survival in breast cancer in the METABRIC database (see, e.g., FIG. 24).

As used herein, the term “decreased of expression” refers to a decrease in mRNA or protein expression levels. For example, the decrease in expression may result from a genetic mutation (e.g., deletion), reduction in gene copy number, decreased transcription, decreased translation or increased degradation at the mRNA or protein level. To establish whether there is a loss/decrease of expression, RNA or protein levels may be compared to a control. For example, comparison may be made to the expression level of EPHX2 from a corresponding non-cancerous cell. Additionally, as described herein, expression may also be normalized using an internal control in certain embodiments.

As used herein, the term “deletion” refers to a reduction in the number of copies of a gene (e.g., hemizygous or homozygous deletion (i.e., null)). In certain embodiments, deletion of EPHX2 results in a decrease in RNA and/or protein expression from that gene.

Accordingly, in certain embodiments, decreased expression of mRNA is detected (e.g., decreased EPHX2 mRNA expression is detected). In certain embodiments, decreased expression of protein is detected (e.g., decreased EPHX2 protein expression is detected). In certain embodiments, deletion of an EPHX2 gene (hemizygous or homozygous deletion) is detected.

Cytochromes P450 (CYPs) are proteins of the superfamily containing heme as a cofactor and, therefore, are hemoproteins. CYPs use a variety of small and large molecules as substrates in enzymatic reactions. They are, in general, the terminal oxidase enzymes in electron transfer chains, broadly categorized as P450-containing systems. Humans have 57 genes and more than 59 pseudogenes divided among 18 families of cytochrome P450 genes and 43 subfamilies, including: CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5A1, CYP7A1, CYP7B1, CYP8A1 (prostacyclin synthase), CYP8B1 (bile acid biosynthesis), CYP11A1, CYP11B1, CYP11B2, CYP17A1, CYP19A1, CYP20A1, CYP21A2, CYP24A1, CYP26A1, CYP26B1, CYP26C1, CYP27A1 (bile acid biosynthesis), CYP27B1 (vitamin D₃ 1-alpha hydroxylase, activates vitamin D₃), CYP27C1 (unknown function), CYP39A1, CYP46A1 and CYP51A1 (lanosterol 14-alpha demethylase).

Accordingly, in certain embodiments, the at least one CYP gene is CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5A1, CYP7A1, CYP7B1, CYP8A1, CYP8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1 and/or CYP51A1. In certain embodiments, the at least one CYP gene is CYP1A1, CYP3A4/5, CYP4F2/3, CYP4F11, CYP4A11, CYP2J2, CYP2C8/9 and/or CYP2S1. In certain embodiments, the CYP gene is CYP3A4/5. In certain embodiments, the CYP gene is CYP4F2/3. In certain embodiments, the CYP gene is CYP2J2. In certain embodiments, the CYP gene is CYP4A11.

In certain embodiments, increased expression of at least one CYP gene is detected/the cancer cell comprises increased expression of CYP. In certain embodiments, expression levels are detected for a panel of CYPs (e.g., 2, 3, 4, 5, 6, 7, 8, 9, 10, 11, 12, 13, 14, 15, 16, 17, 18, 19, 20 or more). In certain embodiments, increased expression of more than one CYP gene is detected/the cancer cell comprises increased expression of more than one CYP (e.g., 2, 3, 4, 5, 6, 7, 8, etc.). In certain embodiments, increased expression of CYP3A4/5 and CYP4F2/3, and optionally, CYP2J2 is detected. In certain embodiments, increased expression of CYP3A4/5 and CYP4F2/3 is detected. In certain embodiments, increased expression of CYP3A4/5, CYP4F2/3, and CYP2J2 is detected. In certain embodiments, increased expression of CYP3A4/5 and CYP2J2 detected. In certain embodiments, there is increased expression of CYP3A4/5 and CYP4A11.

In certain embodiments, expression of at least one CYP is increased by at least about 5%, 10%, 15%, 20%, 30%, 40%, 50%, 60%, 70%, 80%, 90%, 95% or more (e.g., as compared to expression of a corresponding CYP in a corresponding non-cancerous cell).

The epoxide hydrolase 2 (EPHX2) gene, mapping to 8p21.2-p21.1, encodes soluble epoxide hydrolase (sEH), which is a bifunctional enzyme (Gene ID 2053; NG_012964.1). This enzyme, found in both the cytosol and peroxisomes, binds to specific epoxides and converts them to the corresponding diols.

In certain embodiments, decreased expression of EPHX2 is detected/the cancer cell comprises decreased EPHX2 expression. In certain embodiments, expression of EPHX2 is decreased by at least about 5%, 10%, 15%, 20%, 30%, 40%, 50%, 60%, 70%, 80%, 90%, 95% or more (e.g., as compared to expression of EPHX2 in a corresponding non-cancerous cell). In certain embodiments, the EPHX2 gene comprises a mutation, which results in reduced expression (e.g., a frameshift mutation, a missense mutation, a deletion, etc.). In certain embodiments, the EPHX2 gene is deleted (e.g., heterozygous or homozygous deletion). In certain embodiments, EPHX2 expression is decreased by about 50%. In certain embodiments, there is no detectable EPHX2 expression (EPHX2 null).

In certain embodiments, increased CYP expression results in increased CYP epoxygenase activity and/or synthesis of epoxyeicosatrienoic acids (EETs). In certain embodiments, decreased expression of EPHX2 reduces EET hydrolysis.

In certain embodiments, the biguanide compound or other CYP epoxygenase inhibitor inhibits CYP epoxygenase activity and/or inhibits the synthesis of epoxyeicosatrienoic acids (EETs).

In certain embodiments, the cancer is a solid tumor cancer. In certain embodiments, the cancer cell is from a solid tumor. In certain embodiments, the cancer cell is a breast, ovarian, endometrial/uterine, bladder cancer, glioma (e.g., low grade) or lung adenocarcinoma cancer cell. In certain embodiments, the cancer is breast cancer, ovarian cancer, endometrial/uterine cancer, bladder cancer, glioma (e.g., low grade) or lung adenocarcinoma. In certain embodiments, the cancer is breast cancer. In certain embodiments, the breast cancer is ER+. In certain embodiments, the breast cancer is HER2+. In certain embodiments, the breast cancer is triple negative breast cancer (ER−, PR− and HER2−). In certain embodiments, the breast cancer is estrogen positive HER2 negative breast cancer (ER+ HER2−). In certain embodiments, the cancer is ER− breast cancer. In certain embodiments, the cancer is a cancer other than breast cancer. In certain embodiments, the cancer is a cancer other than estrogen positive HER2 negative breast cancer (ER+ HER2−).

In certain embodiments, the cancer is ER− breast cancer. In certain embodiments, the cancer is ER+ breast cancer. In certain embodiments the cancer is estrogen positive HER2 negative breast cancer (ER+ HER2−). In certain embodiments, increased expression of CYP4A11 is detected/the cancer cell comprises increased expression of CYP4A11 (e.g., increased mRNA is detected). As described herein, it has been shown that HBB may inhibit EET production by CYP4A11, but only weakly at 25 to 100 uM. Thus, such a patient would be treated with a CYP expoxygenase inhibitor that decreases EET biosynthesis, such as sesamin (5,5′-(1S,3aR,4S,6aR)-tetrahydro-1H,3H-furo[3,4-c]furan-1,4-diylbis(1,3-benzodioxole)), or a similar drug. In certain other embodiments, increased expression of CYP3A4 is detected/the cancer cell comprises increased expression of CYP3A4 (e.g., increased protein expression). As described herein, it has been shown that HBB may decrease EET production by CYP3A4. Thus, in certain embodiments, a patient having ER+ HER2− breast cancer comprising increased CYP3A4 expression, may be treated with a HBB or another biguanide compound that decreases EET biosynthesis.

In certain embodiments, the method further comprises obtaining a biological sample from a patient for detecting the presence of CYP gene amplification and/or EPHX2 deletion. In certain embodiments, the biological sample is a cancer cell sample. In certain embodiments, a nucleic acid sample (e.g., DNA or mRNA sample) is obtained from the cancer cell sample. In certain embodiments, a protein sample is obtained from the cancer cell sample.

In certain embodiments, the method further comprises detecting increased expression of at least one CYP. In certain embodiments, the method further comprises detecting decreased expression of EPHX2. In certain embodiments, the method further comprises detecting the presence of CYP gene amplification. In certain embodiments, the method further comprises detecting an EPHX2 deletion (homozygous or heterozygous deletion).

In certain embodiments, the method further comprises informing a patient for whom the increased expression of at least one CYP and/or decreased expression of EPHX2 is detected that a biguanide compound or other CYP epoxygenase inhibitor should be administered.

Certain embodiments of the present invention provide kits for practicing methods of the invention, e.g., identifying a cancer cell that is sensitive to a biguanide compound/identifying a patient that can be treated with a biguanide compound. These kits contain packaging material, at least one reagent for detecting expression of at least one CYP and/EPHX2 in a biological sample from the subject, and instructions for its intended use.

Certain embodiments of the invention provide a kit for identifying a cancer cell that is sensitive to a biguanide compound or other CYP epoxygenase inhibitor comprising 1) at least one reagent for detecting increased expression of at least one cytochrome P450 (CYP) and/or decreased expression of soluble epoxide hydrolase (EPHX2) in a cancer cell sample; and 2) instructions for using the reagent, wherein increased expression of at least one CYP and/or decreased expression of EPHX2 indicates the cancer cell is sensitive to a biguanide compound or other CYP epoxygenase inhibitor.

Certain embodiments of the invention provide a kit for identifying a patient having cancer that can be treated with a biguanide compound or other CYP epoxygenase inhibitor comprising 1) at least one reagent for detecting increased expression of at least one cytochrome P450 (CYP) and/or decreased expression of soluble epoxide hydrolase (EPHX2) in a cancer cell sample; and 2) instructions for using the reagent, wherein increased expression of at least one CYP and/or decreased expression of EPHX2 indicates the patient can be treated with a biguanide compound or other CYP epoxygenase inhibitor.

Certain embodiments of the invention provide a kit comprising 1) at least one reagent for detecting increased expression of at least one cytochrome P450 (CYP) and/or decreased expression of soluble epoxide hydrolase (EPHX2) in a cancer cell sample; and 2) instructions for (a) using the reagent to detect increased expression of at least one cytochrome P450 (CYP) gene and/or decreased expression of soluble epoxide hydrolase (EPHX2); and (b) to administer a biguanide compound or other CYP epoxygenase inhibitor to a patient having cancer, wherein increased expression of at least one cytochrome P450 (CYP) and/or decreased expression of soluble epoxide hydrolase (EPHX2) is detected in a cancer cell sample from the patient.

In certain embodiments, the reagent is an oligonucleotide, such as a primer or a probe (e.g., a fluorescent probe). In certain embodiments, the reagent is an antibody.

### Methods for Detecting Gene Amplification and Deletion

A biological sample, according to any of the above methods, may be obtained using certain methods known to those skilled in the art. Biological samples may be obtained from vertebrate animals, and in particular, mammals. Tissue biopsy is often used to obtain a representative piece of tumor tissue. Alternatively, tumor cells can be obtained indirectly in the form of tissues or fluids that are known or thought to contain the tumor cells of interest. Variations in expression (mRNA or protein), target nucleic acids (or encoded polypeptides) and/or gene copy number may be detected from a tumor sample or from other body samples such as urine, sputum or serum. Cancer cells are sloughed off from tumors and appear in such body samples. By screening such body samples, a simple early diagnosis can be achieved for diseases such as cancer. In addition, the progress of therapy can be monitored more easily by testing such body samples for variations in expression, target nucleic acids (or encoded polypeptides) and/or gene copy number. Additionally, methods for enriching a tissue preparation for tumor cells are known in the art. For example, the tissue may be isolated from paraffin or cryostat sections (e.g., formalin-fixed paraffin-embedded (FFPE) tissue). Cancer cells may also be separated from normal cells by flow cytometry or laser capture microdissection.

A nucleic acid, may be e.g., genomic DNA, RNA transcribed from genomic DNA, or cDNA generated from RNA. A nucleic acid or protein may be derived from a vertebrate, e.g., a mammal. A nucleic acid or protein is said to be “derived from” a particular source if it is obtained directly from that source or if it is a copy of a nucleic acid found in that source.

In certain embodiments, genomic DNA is isolated from a biological sample (i.e., comprising cancer cells) and analyzed in the detection assay. In certain embodiments, mRNA is isolated from a biological sample (i.e., comprising cancer cells) and analyzed in the detection assay. In certain embodiments, the methods further comprise reverse transcribing mRNA isolated from the biological sample to generate cDNA.

Variations in nucleic acids and amino acid sequences, as well as gene copy number, may be detected by certain methods known to those skilled in the art. Similarly, nucleic acid expression (e.g., mRNA expression) may be detected using methods known in the art. Such methods include, but are not limited to, polymerase chain reaction (PCR), including quantitative PCR (qPCR) and Real-Time Quantitative Reverse Transcription PCR (qRT-PCR); Northern blot analysis, expression microarray analysis; next generation sequencing (NGS); fluorescence in situ hybridization (FISH); DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan®); and assays employing molecular beacons. Certain of these methods are discussed in further detail below.

In certain embodiments, nucleic acid (e.g., mRNA) expression of CYP and/or EPHX2 is detected using PCR technology. In certain embodiments, nucleic acid expression of CYP and/or EPHX2 is detected using a multiplexed PCR assay. In certain embodiments, nucleic acid expression of CYP and/or EPHX2 is detected using quantitative PCR (qPCR). In certain embodiments, nucleic acid expression of CYP and/or EPHX2 is detected using Real-Time Quantitative Reverse Transcription PCR (qRT-PCR).

In certain embodiments, the nucleic acid is contacted with at least one oligonucleotide probe to form a hybridized nucleic acid. In certain embodiments, the at least one oligonucleotide probe is immobilized on a solid surface. In certain embodiments, the hybridized nucleic acid is amplified. In certain embodiments, the methods further comprise contacting the amplified nucleic acid(s) with a detection oligonucleotide probe, wherein the detection oligonucleotide probe hybridizes to the amplified nucleic acid(s).

In certain embodiments, CYP gene amplification and/or EPHX2 deletion is detected using PCR technology. In certain embodiments, CYP gene amplification and/or EPHX2 deletion is detected using a multiplexed PCR assay. In certain embodiments, CYP gene amplification and/or EPHX2 deletion is detected using quantitative PCR (qPCR). In certain embodiments, CYP gene amplification and/or EPHX2 deletion is detected using Real-Time Quantitative Reverse Transcription PCR (qRT-PCR).

FISH is a cytogenetic technique that uses fluorescent probes that bind to specific parts of the chromosome with a high degree of sequence complementarity. It may be used to detect and localize the presence or absence of specific DNA sequences on chromosomes (e.g., detect copy number variation). Fluorescence microscopy can be used to find out where the fluorescent probe is bound to the chromosomes. FISH can also be used to detect and localize specific RNA targets (e.g., mRNA) in cells.

Accordingly, in certain embodiments, increased CYP mRNA expression and/or decreased EPHX2 mRNA expression is detected using FISH. In certain embodiments, CYP gene amplification and/or EPHX2 deletion is detected using FISH.

In certain embodiments, CYP and/or EPHX2 protein expression is detected. In certain embodiments of the inventions, the methods further comprise isolating protein from the biological sample. Assays for detecting and measuring protein expression are known in the art and include, e.g., western blot analysis, immunofluorescence, immunohistochemistry (e.g., of tissue arrays), etc.

In certain embodiments, increased CYP protein expression and/or decreased expression of EPHX2 protein is detected using western blotting. In certain embodiments, increased CYP protein expression and/or decreased EPHX2 protein expression is detected using immunohistochemistry. In certain embodiments, increased CYP protein expression and/or decreased EPHX2 protein expression is detected using immunofluorescence.

In certain embodiments of the invention, increased CYP protein expression and/or decreased EPHX2 protein expression is detected using an antibody (contacting the biological sample with an antibody). In certain embodiments of the invention, increased CYP protein expression and/or decreased EPHX2 protein expression is detected by contacting a cell from the sample with an antibody. In certain embodiments of the invention, increased CYP protein expression and/or decreased EPHX2 protein expression is detected by contacting proteins isolated from the sample with an antibody. In certain embodiments, the antibody is a CYP or EPHX2 antibody. In certain embodiments, the methods further comprise contacting the sample with a secondary antibody. In certain embodiments, the antibody or secondary antibody is labeled (e.g., with a fluorophore).

In certain embodiments, the expression level of at least one CYP is detected. In certain embodiments, the expression level(s) of 1-10 CYP(s) is detected. In certain embodiments, the expression level(s) of 1-5 CYP(s) is detected. In certain embodiments, the expression levels of 3-5 CYPs are detected. In certain embodiments, the copy number of at least one CYP gene is detected. In certain embodiments, the copy number of 1-10 CYP genes is detected. In certain embodiments, the copy number of 1-5 CYP genes is detected. In certain embodiments, the copy number of 3-5 CYP genes is detected.

In certain embodiments, normalization controls are used in the detection assay (e.g., a housekeeping gene, such as GAPDH, beta actin, ribosomal protein genes, RPLPO, GUS, a cytokeratin (e.g., cytokeratin 8) or TFRC). Accordingly, in certain embodiments, the expression level of CYP and/EPHX2 protein or RNA in the biological sample is normalized to the level of a control protein or RNA in the biological sample.

In certain embodiments, expression levels may be compared to expression levels from a control cell/sample to establish whether expression is increased or decreased. For example, expression may be compared to expression of a corresponding CYP from a corresponding non-cancerous cell (e.g., expression of CYP3A4 from a breast cancer cell could be compared to the expression of CYP3A4 from non-cancerous breast cell).

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom. Amplification techniques are well known in the art, e.g., polymerase chain reaction is described in Saiki et al., Science 239:487, 1988; U.S. Pat. Nos. 4,683,203 and 4,683,195.

The ligase chain reaction, which is known in the art, can also be used to amplify target nucleic acid sequences. see, e.g., Wu et al., Genomics 4:560-569 (1989). In addition, a technique known as allele-specific PCR can also be used to detect variations (e.g., substitutions). see, e.g., Ruano and Kidd (1989) Nucleic Acids Research 17:8392; McClay et al. (2002) Analytical Biochem. 301:200-206. In certain embodiments of this technique, an allele-specific primer is used wherein the 3′ terminal nucleotide of the primer is complementary to (i.e., capable of specifically base-pairing with) a particular variation in the target nucleic acid. If the particular variation is not present, an amplification product is not observed. Amplification Refractory Mutation System (ARMS) can also be used to detect variations (e.g., substitutions). ARMS is described, e.g., in European Patent Application Publication No. 0332435, and in Newton et al., Nucleic Acids Research, 17:7, 1989.

Other methods useful for detecting variations (e.g., substitutions) include, but are not limited to, (1) allele-specific nucleotide incorporation assays, such as single base extension assays (see, e.g., Chen et al. (2000) Genome Res. 10:549-557; Fan et al. (2000) Genome Res. 10:853-860; Pastinen et al. (1997) Genome Res. 7:606-614; and Ye et al. (2001) Hum. Mut. 17:305-316); (2) allele-specific primer extension assays (see, e.g., Ye et al. (2001) Hum. Mut. 17:305-316; and Shen et al. Genetic Engineering News, vol. 23, Mar. 15, 2003), including allele-specific PCR; (3) 5′nuclease assays (see, e.g., De La Vega et al. (2002) BioTechniques 32:S48-S54 (describing the TaqMan® assay); Ranade et al. (2001) Genome Res. 11:1262-1268; and Shi (2001) Clin. Chem. 47:164-172); (4) assays employing molecular beacons (see, e.g., Tyagi et al. (1998) Nature Biotech. 16:49-53; and Mhlanga et al. (2001) Methods 25:463-71); and (5) oligonucleotide ligation assays (see, e.g., Grossman et al. (1994) Nuc. Acids Res. 22:4527-4534; patent application Publication No. US 2003/0119004 A1; PCT International Publication No. WO 01/92579 A2; and U.S. Pat. No. 6,027,889).

Variations may also be detected by mismatch detection methods. Mismatches are hybridized nucleic acid duplexes which are not 100% complementary. The lack of total complementarity may be due to deletions, insertions, inversions, or substitutions. One example of a mismatch detection method is the Mismatch Repair Detection (MRD) assay described, e.g., in Faham et al., Proc. Natl Acad. Sci. USA 102:14717-14722 (2005) and Faham et al., Hum. Mol. Genet. 10:1657-1664 (2001). Another example of a mismatch cleavage technique is the RNase protection method, which is described in detail in Winter et al., Proc. Natl. Acad. Sci. USA, 82:7575, 1985, and Myers et al., Science 230:1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid, but can a portion of the target nucleic acid, provided it encompasses the position suspected of having a variation.

In a similar manner, DNA probes can be used to detect mismatches, for example through enzymatic or chemical cleavage. see, e.g., Cotton et al., Proc. Natl. Acad. Sci. USA, 85:4397, 1988; and Shenk et al., Proc. Natl. Acad. Sci. USA, 72:989, 1975. Alternatively, mismatches can be detected by shifts in the electrophoretic mobility of mismatched duplexes relative to matched duplexes. see, e.g., Cariello, Human Genetics, 42:726, 1988. With either riboprobes or DNA probes, the target nucleic acid suspected of comprising a variation may be amplified before hybridization. Changes in target nucleic acid can also be detected using Southern hybridization, especially if the changes are gross rearrangements, such as deletions and insertions.

Restriction fragment length polymorphism (RFLP) probes for the target nucleic acid or surrounding marker genes can be used to detect variations, e.g., insertions or deletions. Insertions and deletions can also be detected by cloning, sequencing and amplification of a target nucleic acid. Single stranded conformation polymorphism (SSCP) analysis can also be used to detect base change variants of an allele. see, e.g., Orita et al., Proc. Natl. Acad. Sci. USA 86:2766-2770, 1989, and Genomics, 5:874-879, 1989.

### Biguanide Compounds and Other CYP Epoxygenase Inhibitors

As used herein, the term “CYP epoxygenase inhibitor” refers to any compound or treatment that inhibits CYP epoxygenase activity. In certain embodiments, the inhibitor is a small molecule. The term “small molecule” includes organic molecules having a molecular weight of less than about 1000 amu. In one embodiment a small molecule can have a molecular weight of less than about 800 amu. In another embodiment a small molecule can have a molecular weight of less than about 500 amu. Examples of CYP expoygenase inhibitors include, but are not limited to, biguanide compounds, telmisartin, sesamin (5,5′-(1S,3aR,4S,6aR)-tetrahydro-1H,3H-furo[3,4-c]furan-1,4-diylbis(1,3-benzodioxole)), HET0016 and dibromododecenyl methylsulfonimide (DDMS).

Biguanide compounds are known in the art and comprise the structural group:

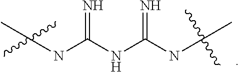 [SMILES: *NC(=N)NC(=N)N*]

In one embodiment, the biguanide compound comprises structural group:

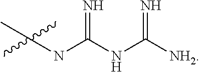 [SMILES: *NC(=N)NC(=N)N]

In certain embodiments, the biguanide compound has a molecular weight below about 500 amu. In certain embodiments, the biguanide compound has a molecular weight below 400 amu.

In certain embodiments, the biguanide compound inhibits CYP epoxygenase activity and/or inhibits the synthesis of epoxyeicosatrienoic acids (EETs).

In certain embodiments, the biguanide compound is a compound described in US Patent Publication No. 2015/0342909, which is incorporated by reference in its entirety.

In certain embodiments, the biguanide compound is metformin, buformin or phenformin. In certain embodiments, the biguanide compound is metformin.

In certain embodiments, the biguanide compound is hexyl-benzyl-biguanide (HBB).

In certain embodiments, the biguanide compound is a compound of formula I:

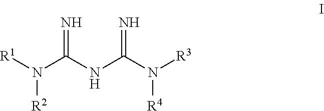

wherein:

R¹ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl, or —O(C₂-C₁₂)alkynyl of R¹ is optionally substituted with one or more (e.g., 1, 2, 3, 4, 5 or more) Z^{1a} groups and wherein any (C₃-C₈)carbocycle, 5-10 membered heteroaryl or aryl of R¹ is optionally substituted with one or more (e.g., 1, 2, 3, 4, 5 or more) Z^{1b} groups;

R² is H, (C₁-C₁₂)alkyl, (C₂-C₁₂)alkenyl, (C₂-C₁₂)alkynyl, —O(C₂-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl, or —O(C₂-C₁₂)alkynyl of R² is optionally substituted with one or more (e.g., 1, 2, 3, 4, 5 or more) Z^{2a} groups and wherein any (C₃-C₈)carbocycle, 5-10 membered heteroaryl or aryl of R¹ is optionally substituted with one or more (e.g., 1, 2, 3, 4, 5 or more) Z^{2b} groups;

R³ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl or —O(C₂-C₁₂)alkynyl of R³ is optionally substituted with one or more (e.g., 1, 2, 3, 4, 5 or more) Z^{3a} groups and wherein any (C₃-C₈)carbocycle, 5-10 membered heteroaryl or aryl of R³ is optionally substituted with one or more (e.g., 1, 2, 3, 4, 5 or more) Z^{3b} groups;

R⁴ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl or —O(C₂-C₁₂)alkynyl of R⁴ is optionally substituted with one or more (e.g., 1, 2, 3, 4, 5 or more) Z^{4a} groups and wherein any (C₃-C₈)carbocycle, 5-10 membered heteroaryl or aryl of R⁴ is optionally substituted with one or more (e.g., 1, 2, 3, 4, 5 or more) Z^{4b} groups;

Z^{1a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{1a} is optionally substituted with one or more (e.g., 1, 2, 3, 4, 5 or more) groups selected from (C₁-C₆)alkyl, —OH, halogen and —O(C₁-C₆)alkyl;

Z^{1b} is (C₁-C₆)alkyl, —OH, halogen or —O(C₁-C₆)alkyl;

Z^{2a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{2a} is optionally substituted with one or more (e.g., 1, 2, 3, 4, 5 or more) groups selected from (C₁-C₆)alkyl, —OH, halogen and —O(C₁-C₆)alkyl;

Z^{2b} is (C₁-C₆)alkyl, —OH, halogen or —O(C₁-C₆)alkyl;

Z^{3a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{3a} is optionally substituted with one or more (e.g., 1, 2, 3, 4, 5 or more) groups selected from (C₁-C₆)alkyl, —OH, halogen and —O(C₁-C₆)alkyl;

Z^{3b} is (C₁-C₆)alkyl, —OH, halogen or —O(C₁-C₆)alkyl;

Z^{4a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{4a} is optionally substituted with one or more (e.g., 1, 2, 3, 4, 5 or more) groups selected from (C₁-C₆)alkyl, —OH, halogen and —O(C₁-C₆)alkyl; and

Z^{4b} is (C₁-C₆)alkyl, —OH, halogen or —O(C₁-C₆)alkyl;

or a pharmaceutically acceptable salt thereof.

A specific value for R² is H.

A specific value for R⁴ is H.

A specific group of compounds of formula I are compounds of formula Ia:

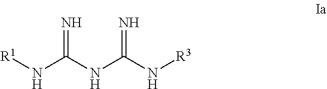

or a salt thereof.

A specific value for R¹ is (C₁-C₁₂)alkyl, (C₂-C₁₂)alkenyl, or (C₂-C₁₂)alkynyl, wherein any (C₁-C₁₂)alkyl, (C₂-C₁₂)alkenyl or (C₂-C₁₂)alkynyl of R¹ is optionally substituted with one or more Z^{1a} groups.

A specific value for R¹ is (C₁-C₁₂)alkyl, (C₂-C₁₂)alkenyl or (C₂-C₁₂)alkynyl.

A specific value for R¹ is (C₄-C₈)alkyl, (C₄-C₈)alkenyl or (C₄-C₈)alkynyl.

A specific value for R¹ is (C₁-C₁₂)alkyl.

A specific value for R¹ is (C₂-C₁₀)alkyl.

A specific value for R¹ is (C₄-C₈)alkyl.

A specific value for R¹ is (C₆)alkyl.

A specific value for R¹ is hexyl.

A specific value for R⁴ is (C₁-C₁₂)alkyl, (C₂-C₁₂)alkenyl, (C₂-C₁₂)alkynyl, wherein any (C₁-C₁₂)alkyl, (C₂-C₁₂)alkenyl or (C₂-C₁₂)alkynyl of R⁴ is optionally substituted with one or more Z^{4a} groups.

A specific value for R⁴ is (C₁-C₁₂)alkyl, (C₂-C₁₂)alkenyl, (C₂-C₁₂)alkynyl, wherein any (C₁-C₁₂)alkyl, (C₂-C₁₂)alkenyl or (C₂-C₁₂)alkynyl of R⁴ is substituted with one or more Z^{4a} groups.

A specific value for R⁴ is (C₁-C₄)alkyl, (C₂-C₄)alkenyl, (C₂-C₄)alkynyl, wherein any (C₁-C₄)alkyl, (C₂-C₄)alkenyl or (C₂-C₄)alkynyl of R⁴ is substituted with one or more Z^{4a} groups.

A specific value for R⁴ is (C₁-C₆)alkyl, wherein any (C₁-C₆)alkyl of R⁴ is substituted with one or more Z^{4a} groups.

A specific value for R⁴ is (C₁-C₃)alkyl, wherein any (C₁-C₃)alkyl of R⁴ is substituted with one or more Z^{4a} groups.

A specific value for R⁴ is —CH₂—Z^{4a}.

A specific group of compounds of formula I are compounds of formula Ib:

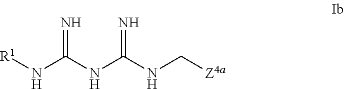

or a salt thereof.

A specific value for Z^{4a} is (C₃-C₈)carbocycle, 5-10 membered heteroaryl or aryl, wherein any (C₃-C₈)carbocycle, 5-10 membered heteroaryl or aryl of Z^{4a} is optionally substituted with one or more groups selected from (C₁-C₆)alkyl, —OH, halogen and —O(C₁-C₆)alkyl.

A specific value for Z^{4a} is 5-10 membered heteroaryl or aryl, wherein any 5-10 membered heteroaryl or aryl of Z^{4a} is optionally substituted with one or more groups selected from (C₁-C₆)alkyl, —OH, halogen and —O(C₁-C₆)alkyl.

A specific value for Z^{4a} is a 5 membered heteroaryl, 6 membered heteroaryl or phenyl, wherein any 5 membered heteroaryl, 6 membered heteroaryl or phenyl of Z^{4a} is optionally substituted with one or more groups selected from (C₁-C₆)alkyl, —OH, halogen and —O(C₁-C₆)alkyl.

A specific value for Z^{4a} is a 5 membered heteroaryl, 6 membered heteroaryl or phenyl.

A specific compound of formula I is:

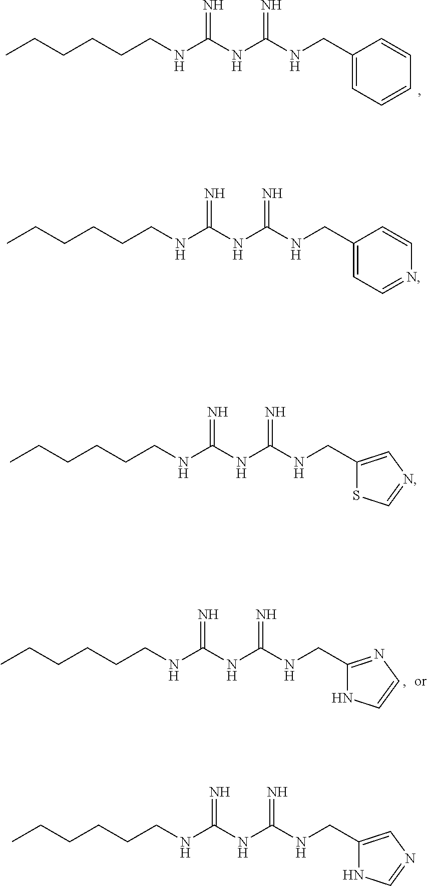 [SMILES: CCCCCCNC(=N)NC(=N)NCc1ccccc1 ; CCCCCCNC(=N)NC(=N)NCc1ccncc1 ; CCCCCCNC(=N)NC(=N)NCc1cncs1 ; CCCCCCNC(=N)NC(=N)NCc1ncc[nH]1 ; CCCCCCNC(=N)NC(=N)NCc1cnc[nH]1]

or a salt thereof.

In one embodiment the compound of formula I is metformin or a pharmaceutically acceptable salt thereof.

In one embodiment the compound of formula I does not include metformin.

A specific group of compounds of formula I are compounds of formula Ib:

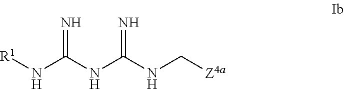

wherein

R¹ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl of R¹ is optionally substituted with one or more Z^{1a} groups and wherein any (C₃-C₈)carbocycle, 5-10 membered heteroaryl or aryl of R¹ is optionally substituted with one or more Z^{1b} groups; and

Z^{1a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{1a} is optionally substituted with one or more groups selected from (C₁-C₆)alkyl, —OH, halogen and —O(C₁-C₆)alkyl;

Z^{1b} is (C₁-C₆)alkyl, —OH, halogen or —O(C₁-C₆)alkyl; and

Z^{4a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{4a} is optionally substituted with one or more groups selected from (C₁-C₆)alkyl, —OH, halogen and —O(C₁-C₆)alkyl;

or a salt thereof.

A specific value for R¹ is (C₁-C₁₂)alkyl, (C₂-C₁₂)alkenyl or (C₂-C₁₂)alkynyl, wherein any (C₁-C₁₂)alkyl, (C₂-C₁₂)alkenyl or (C₂-C₁₂)alkynyl of R¹ is optionally substituted with one or more Z^{1a} groups.

A specific value for R¹ is (C₁-C₁₂)alkyl, (C₂-C₁₂)alkenyl or (C₂-C₁₂)alkynyl.

A specific value for R¹ is (C₄-C₈)alkyl, (C₄-C₈)alkenyl or (C₄-C₈)alkynyl.

A specific value for R¹ is (C₁-C₁₂)alkyl.

A specific value for R¹ is (C₂-C₁₀)alkyl.

A specific value for R¹ is (C₄-C₈)alkyl.

A specific value for R¹ is (C₆)alkyl.

A specific value for R¹ is hexyl.

A specific value for R¹ is n-hex-1-yl.

A specific value for R¹ —(CH₂)₅CH₃.

A specific value for Z^{4a} is 5-10 membered heteroaryl, wherein any 5-10 membered heteroaryl or aryl of Z^{4a} is optionally substituted with one or more groups selected from (C₁-C₆)alkyl, —OH, halogen and —O(C₁-C₆)alkyl.

A specific value for Z^{4a} is a 5 membered heteroaryl or 6 membered heteroaryl, wherein any 5 membered heteroaryl or 6 membered heteroaryl of Z^{4a} is optionally substituted with one or more groups selected from (C₁-C₆)alkyl, —OH, halogen and —O(C₁-C₆)alkyl.

A specific value for Z^{4a} is a 5 membered heteroaryl or 6 membered heteroaryl.

A specific value for Z^{4a} is imidazolyl, pyridinyl or thiazolyl, wherein any imidazolyl, pyridinyl or thiazolyl of Z^{4a} is optionally substituted with one or more groups selected from (C₁-C₆)alkyl, —OH, halogen and —O(C₁-C₆)alkyl.

A specific value for Z^{4a} is imidazolyl, pyridinyl or thiazolyl.

A specific value for Z^{4a} is:

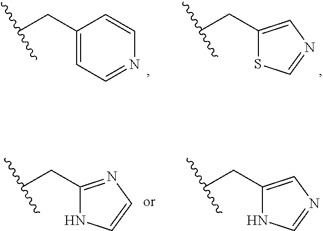 [SMILES: *Cc1ccncc1 ; *Cc1cncs1 ; *Cc1ncc[nH]1 ; *Cc1cnc[nH]1]

A compound selected from:

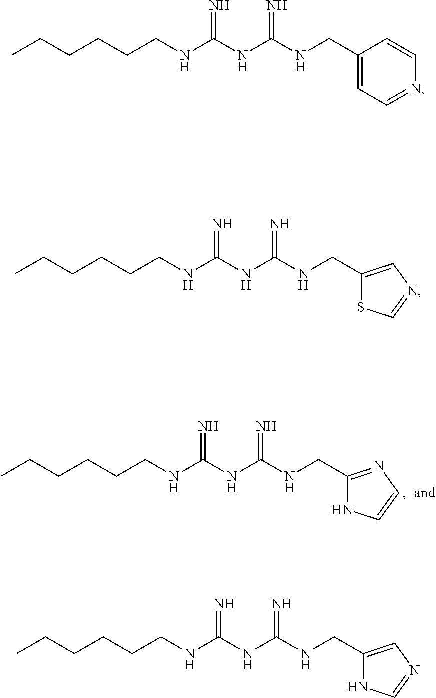 [SMILES: CCCCCCNC(=N)NC(=N)NCc1ccncc1 ; CCCCCCNC(=N)NC(=N)NCc1cncs1 ; CCCCCCNC(=N)NC(=N)NCc1ncc[nH]1 ; CCCCCCNC(=N)NC(=N)NCc1cnc[nH]1]

and salts thereof.

In certain embodiments, the biguanide compound is selected from the group consisting of:

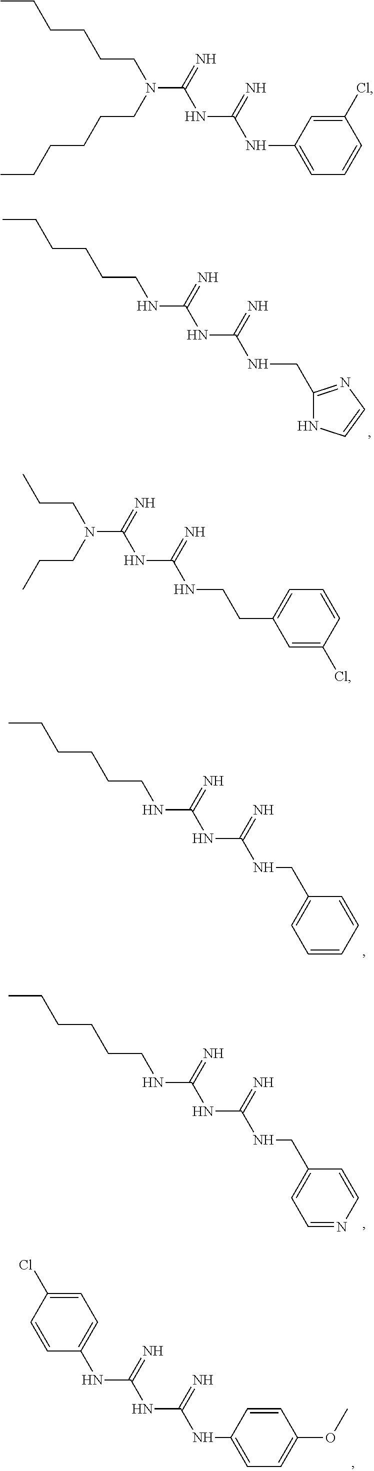

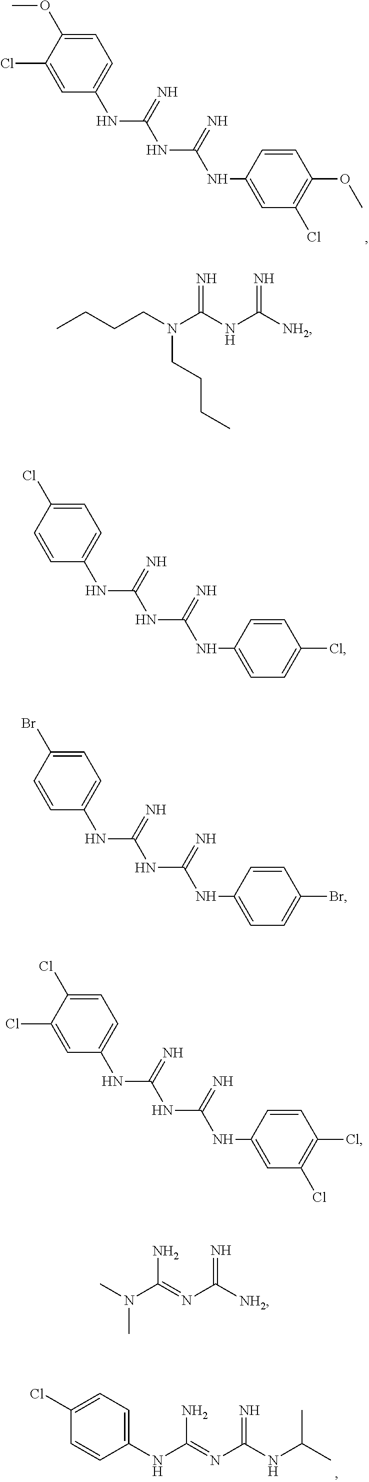

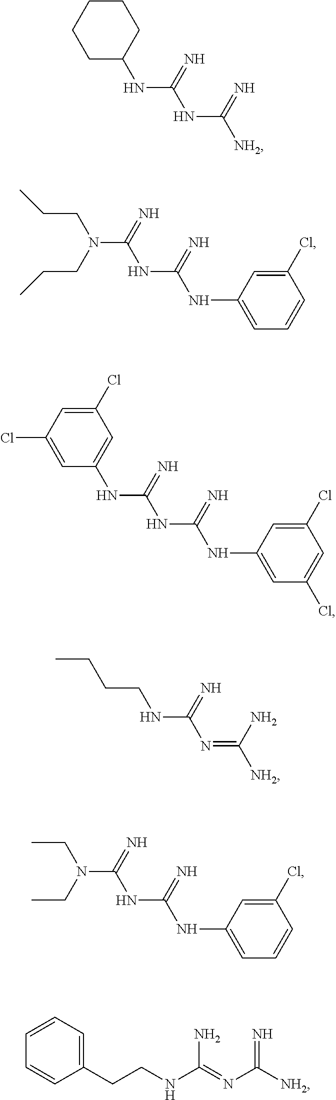 [SMILES: N=C(N)NC(=N)NC1CCCCC1 ; CCCN(CCC)C(=N)NC(=N)Nc1cccc(Cl)c1 ; N=C(NC(=N)Nc1cc(Cl)cc(Cl)c1)Nc1cc(Cl)cc(Cl)c1 ; CCCCNC(=N)N=C(N)N ; CCN(CC)C(=N)NC(=N)Nc1cccc(Cl)c1 ; N=C(N)/N=C(\N)NCCc1ccccc1]

and salts thereof.

In one embodiment a salt is a pharmaceutically acceptable salt.

Administration of a biguanide compound as a pharmaceutically acceptable acid or base salt may be appropriate. Examples of pharmaceutically acceptable salts include organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic acid addition salts may also be formed, which include a physiological acceptable anion, for example, chloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Definitions

The following definitions are used, unless otherwise described.

The term “alkyl” is a straight or branched saturated hydrocarbon. For example, an alkyl group can have 1 to 8 carbon atoms (i.e., (C₁-C₈)alkyl) or 1 to 6 carbon atoms (i.e., (C₁-C₆ alkyl) or 1 to 4 carbon atoms.

The term “alkenyl” is a straight or branched hydrocarbon with at least one carbon-carbon double bond. For example, an alkenyl group can have 2 to 8 carbon atoms (i.e., C₂-C₈ alkenyl), or 2 to 6 carbon atoms (i.e., C₂-C₆ alkenyl). Examples of suitable alkenyl groups include, but are not limited to, ethylene or vinyl (—CH═CH₂), allyl (—CH₂CH═CH₂) and 5-hexenyl (—CH₂CH₂CH₂CH₂CH═CH₂).

The term “alkynyl” is a straight or branched hydrocarbon with at least one carbon-carbon triple bond. For example, an alkynyl group can have 2 to 8 carbon atoms (i.e., C₂-C₈ alkyne,), or 2 to 6 carbon atoms (i.e., C₂-C₆ alkynyl). Examples of suitable alkynyl groups include, but are not limited to, acetylenic (—C≡CH), propargyl (—CH₂C≡CH), and the like.

The term “halo” or “halogen” as used herein refers to fluoro, chloro, bromo and iodo.

The term “haloalkyl” as used herein refers to an alkyl as defined herein, wherein one or more hydrogen atoms are each replaced by a halo substituent. For example, a (C₁-C₆)haloalkyl is a (C₁-C₆)alkyl wherein one or more of the hydrogen atoms have been independently replaced by a halo substituent. Such a range includes one halo substituent on the alkyl group to complete halogenation of the alkyl group. The halo substituents may be the same or different.

The term “carbocycle” or “carbocyclyl” refers to a single saturated (i.e., cycloalkyl) or a single partially unsaturated (e.g., cycloalkenyl, cycloalkadienyl, etc.) all carbon ring having for example 3 to 8 carbon atoms (i.e., (C₃-C₈)carbocycle) or 3 to 7 carbon atoms (i.e., (C₃-C₇)carbocycle). The term “carbocycle” or “carbocyclyl” also includes multiple condensed, saturated and partially unsaturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). The “carbocycle” or “carbocyclyl” can also be optionally substituted with one or more (e.g., 1, 2 or 3) oxo groups. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

The term “aryl” as used herein refers to a single all carbon aromatic ring or a multiple condensed all carbon ring system wherein at least one of the rings is aromatic. For example, an aryl group can have 6 to 20 carbon atoms, 6 to 14 carbon atoms, or 6 to 12 carbon atoms or 6 to 10 carbon atoms. Aryl includes a phenyl radical. Aryl also includes multiple condensed ring systems (e.g., ring systems comprising 2 or 3 rings) having about 9 to 20 carbon atoms in which at least one ring is aromatic and wherein the other rings may be aromatic or not aromatic (i.e., carbocycle). Such multiple condensed ring systems may be optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Typical aryl groups include, but are not limited to phenyl, indenyl, naphthyl, 1,2,3,4-tetrahydronaphthyl, anthracenyl, and the like.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system such as a 5-10 membered heteroaryl) has about 1-9 carbon atoms and about 1-4 heteroatoms within the heteroaryl ring; or a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system. It is also to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5-14 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclopenta[1,2-c]pyrazole.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase. It is to be understood that all rotational isomers for compounds of formula I, Ia and Ib are within the scope of the invention.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that one or more values may be combined.

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

“Oligonucleotide probe” can refer to a nucleic acid segment, such as a primer, that may be useful to amplify a sequence in the nucleic acid of interest (e.g., CYP DNA, RNA, mRNA or cDNA; EPHX2 DNA, RNA, mRNA or cDNA) and that is complementary to, and hybridizes specifically to, a particular sequence in the nucleic acid of interest.

The term “primer” refers to a single stranded polynucleotide that is capable of hybridizing to a nucleic acid and allowing the polymerization of a complementary nucleic acid, generally by providing a free 3′-OH group.

The term “nucleotide variation” refers to a change in a nucleotide sequence (e.g., an insertion, deletion, inversion, or substitution of one or more nucleotides, such as a single nucleotide polymorphism (SNP)) relative to a reference sequence (e.g., a wild type sequence). The term also encompasses the corresponding change in the complement of the nucleotide sequence, unless otherwise indicated. A nucleotide variation may be a somatic mutation or a germline polymorphism.

The term “copy number” or “copy number variant” refers to the number of copies of a particular gene in the genotype of an individual.

The term “amino acid variation” refers to a change in an amino acid sequence (e.g., an insertion, substitution, or deletion of one or more amino acids, such as an internal deletion or an N- or C-terminal truncation) relative to a reference sequence (e.g., a wild type sequence).

As used herein, the term “specifically hybridizes” or “specifically detects” refers to the ability of a nucleic acid molecule to hybridize to at least approximately six consecutive nucleotides of a sample nucleic acid.

The terms “protein,” “peptide” and “polypeptide” are used interchangeably herein.

In the context of the present invention, an “isolated” or “purified” nucleic acid molecule is a molecule that, by human intervention, exists apart from its native environment. An isolated nucleic acid molecule may exist in a purified form or may exist in a non-native environment. For example, an “isolated” or “purified” nucleic acid molecule, or portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences and proteins or partial-length proteins encoded thereby are also encompassed by the present invention.

By “fragment” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment” means a sequence having, for example, at least 80 nucleotides, at least 150 nucleotides, or at least 400 nucleotides. If not employed for expressing, a “portion” or “fragment” means, for example, at least 9, 12, 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, to 99% sequence identity to the native (endogenous) nucleotide sequence.

“Synthetic” polynucleotides are those prepared by chemical synthesis.

“Recombinant nucleic acid molecule” is a combination of nucleic acid sequences that are joined together using recombinant DNA technology and procedures used to join together DNA sequences as described, for example, in Sambrook and Russell (2001).

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

“Somatic mutations” are those that occur only in certain tissues, e.g., in liver tissue, and are not inherited in the germline. “Germline” mutations can be found in any of a body's tissues and are inherited.

As used herein, the term “control sample” refers to a biological sample from a subject that does not have cancer.

As used herein, the phrase “control protein or RNA” can refer to a protein or RNA whose expression remains constant and is not affected by cancer. In certain embodiments, the control protein or RNA is encoded by a housekeeping gene, for example, GAPDH, beta actin, ribosomal protein genes, RPLPO, GUS, a cytokeratin (e.g., cytokeratin 8) or TFRC.

The term “biomarker” is generally defined herein as a biological indicator, such as a particular molecular feature, that may affect or be related to diagnosing or predicting an individual's health.

The term “detection” includes any means of detecting, including direct and indirect detection.

The term “diagnosis” is used herein to refer to the identification or classification of a molecular or pathological state, disease or condition. For example, “diagnosis” may refer to identification of a particular type of cancer, e.g., breast cancer. “Diagnosis” may also refer to the classification of a particular type of cancer, e.g., by histology (e.g., a non small cell lung carcinoma), by molecular features (e.g., a lung cancer characterized by nucleotide and/or amino acid variation(s) in a particular gene or protein), or both.

The term “prognosis” is used herein to refer to the prediction of the likelihood of cancer-attributable death or progression, including, for example, recurrence, metastatic spread, and drug resistance, of a neoplastic disease, such as cancer.

The term “prediction” or (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The terms “cancer” and “cancerous” refer to or describe the physiological condition in mammals that is typically characterized by unregulated cell growth and proliferation. Examples of cancer include, but are not limited to, carcinoma, lymphoma (e.g., Hodgkin's and non-Hodgkin's lymphoma), blastoma, sarcoma, and leukemia. More particular examples of cancers include squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

The term “treat”, “treatment” or “treating,” to the extent it relates to a disease or condition includes inhibiting the disease or condition, eliminating the disease or condition, and/or relieving one or more symptoms of the disease or condition.

The term “patient” as used herein refers to any animal including mammals such as humans, higher non-human primates, rodents domestic and farm animals such as cow, horses, dogs and cats. In one embodiment, the patient is a human patient.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “long-term” survival is used herein to refer to survival for at least 1 year, 5 years, 8 years, or 10 years following therapeutic treatment.

The terms “obtaining a sample from a patient”, “obtained from a patient” and similar phrasing, is used to refer to obtaining the sample directly from the patient, as well as obtaining the sample indirectly from the patient through an intermediary individual (e.g., obtaining the sample from a courier who obtained the sample from a nurse who obtained the sample from the patient).

### Administration

A biguanide compound or other CYP epoxygenase inhibitor can be formulated as pharmaceutical composition and administered to a mammalian host, such as a human patient in a variety of forms adapted to the chosen route of administration, i.e., orally or parenterally, by intravenous, intramuscular, topical or subcutaneous routes.

Biguanide compounds other CYP epoxygenase inhibitors can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of biguanide compounds other CYP epoxygenase inhibitors can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound is conveniently formulated in unit dosage form. In one embodiment, the invention provides a composition comprising a compound of the invention formulated in such a unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Biguanide compounds other CYP epoxygenase inhibitors can also be administered in combination with other therapeutic agents, for example, other agents that are useful for treating cancer. Examples of such agents include chemotherapeutic agents or radiation therapies.

Accordingly, one embodiment the invention also provides for the use of a composition comprising a biguanide compound other CYP epoxygenase inhibitor, or a pharmaceutically acceptable salt thereof, at least one other therapeutic agent, and a pharmaceutically acceptable diluent or carrier. The invention also provides a kit comprising a biguanide compound other CYP epoxygenase inhibitor, or a pharmaceutically acceptable salt thereof, at least one other therapeutic agent, packaging material, and instructions for administering the biguanide compound or other CYP epoxygenase inhibitor, and the other therapeutic agent or agents to an animal to treat cancer.

### Certain Embodiments for the Treatment of Cancer

Certain embodiments of the invention provide a method for treating cancer in a mammal comprising administering an effective amount of 1) hexyl-benzyl-biguanide (HBB), or a pharmaceutically acceptable salt thereof; and 2) paclitaxel to the mammal.

HBB and paclitaxel may be administered either simultaneously or sequentially. In certain embodiments, HBB is administered simultaneously with paclitaxel. In certain embodiments, a composition (e.g., a pharmaceutical composition) comprising HBB and paclitaxel is administered. In certain embodiments, HBB and paclitaxel are administered sequentially. In certain embodiments, the HBB is administered first and paclitaxel is administered second. In certain embodiments, the paclitaxel is administered first and HBB is administered second.

Certain embodiments of the invention provide hexyl-benzyl-biguanide (HBB), or a pharmaceutically acceptable salt thereof, and paclitaxel for the prophylactic or therapeutic treatment of a cancer.

Certain embodiments of the invention provide a combination comprising hexyl-benzyl-biguanide (HBB), or pharmaceutically acceptable salt thereof, and paclitaxel for the prophylactic or therapeutic treatment of a cancer.

Certain embodiments of the invention provide the use of hexyl-benzyl-biguanide (HBB), or a pharmaceutically acceptable salt thereof, and paclitaxel to prepare a medicament for treating cancer in an animal (e.g. a mammal such as a human).

Certain embodiments of the invention provide a pharmaceutical composition comprising hexyl-benzyl-biguanide (HBB), or pharmaceutically acceptable salt thereof, and paclitaxel.

Certain embodiments of the invention provide a pharmaceutical composition comprising hexyl-benzyl-biguanide (HBB), or a pharmaceutically acceptable salt thereof, and paclitaxel for the prophylactic or therapeutic treatment of cancer.

Certain embodiments of the invention provide a kit comprising hexyl-benzyl-biguanide (HBB), or a pharmaceutically acceptable salt thereof, and paclitaxel, packaging material, and instructions for administering HBB, or a pharmaceutically acceptable salt thereof, and paclitaxel to a mammal to treat cancer.

In certain embodiments, the cancer is carcinoma, lymphoma (e.g., Hodgkin's and non-Hodgkin's lymphoma), blastoma, sarcoma, or leukemia. In certain embodiments, the cancer is a solid tumor cancer.

In certain embodiments, the cancer is squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia, or head and neck cancer. In certain embodiments, the cancer is a breast, ovarian, endometrial/uterine, bladder, glioma (e.g., low grade) or lung adenocarcinoma cancer. In certain embodiments, the cancer is ovarian, endometrial/uterine, bladder, glioma (e.g., low grade) or lung adenocarcinoma cancer. In certain embodiments, the cancer is a cancer other than breast cancer (e.g., other than estrogen positive HER2 negative breast cancer (ER+ HER2−).

The invention will now be illustrated by the following non-limiting Examples.

### Example 1

### Heme Binding Biguanides Target Cytochrome P450 Dependent Cancer Cell Mitochondria

How cytochrome P450 monooxygenases promote cancer is unknown and they remain to be validated as therapeutic targets. The monooxygenase CYP3A4 was found to be associated with breast cancer cell mitochondria. CYP3A4 synthesized epoxyeicosatrienoic acids (EETs), which promoted the mitochondrial membrane potential and oxygen consumption. EETs inhibited AMPK, suggesting that CYP3A4 tonically suppresses catabolism through EET biosynthesis. CYP3A4 knockdown promoted catabolism and prevented mammary tumor growth, thereby validating CYP3A4 as metabolic switch and therapeutic target. The AMPK activator metformin inhibited CYP3A4-mediated EET biosynthesis and bound to the active site heme in a co-crystal structure. N1-hexyl-N5-benzyl-biguanide (HBB) bound the CYP3A4 heme more tightly and potently inhibited epoxygenase activity (Kᵢ=9 μM). HBB rapidly activated AMPK, while inhibiting mTOR, effectively inhibiting ER+ breast tumor growth (24 mg/kg/week) and intratumoral mTOR. CYP suppression of AMPK and catabolism through EET biosynthesis thereby reveals a novel metabolic regulatory pathway in cancer that is susceptible to biguanide inhibition.

### Introduction

Cytochrome P450 metabolism of polyunsaturated fatty acids is required for breast cancer cell proliferation; however, a lack of mechanistic understanding of the role of this pathway in tumor growth has prevented development of targeted therapies. Recently, roles were discovered for arachidonic acid (AA) epoxides, called epoxyeicosatrienoic acids (EETs), in the proliferation, mitogenesis and survival of ER+HER2− breast cancer cells (Mitra et al., J Biol Chem. 2011; 286:17543-59). Furthermore, exogenous EETs rescue these phenotypes in CYP3A4 knockdown breast cancer cells in part by activating Stat3 (Mitra et al., J Biol Chem. 2011; 286:17543-59), but conceptually, EETs have not been linked to mitochondrial function in cancer cells. Nonetheless, current knowledge suggests that EET synthesizing cytochrome P450 (CYP) epoxygenase enzymes could be novel targets for breast cancer therapeutics. Certain CYPs, such as CYP2J2, CYP2C8, CYP3A4, and others are known to synthesize EETs and have been linked to cancer progression (Mitra et al., J Biol Chem. 2011; 286:17543-59; Jiang et al., Cancer Res. 2005; 65:4707-15; Jiang et al., Cancer Res. 2007; 67:6665-74; Pozzi et al., J Biol Chem. 2010; 285:12840-50; Panigrahy et al., J Clin Invest. 2012; 122:178-91).

Effective chemical inhibitors of CYP3A4 epoxygenase activity are needed to elucidate mechanisms by which EETs promote breast cancer growth (Mitra et al., J Biol Chem. 2011; 286:17543-59). While gene silencing can provide important information implicating CYP3A4 in cancer growth, additional information about mechanism can be found using chemical probes that inhibit epoxygenase activity. To develop chemical probes, repurposing approved drugs of differing classes that are known to inhibit certain CYP3A4 activities were first considered, such as the biguanide diabetes drug metformin, which inhibits metabolism of nifedipine, a calcium channel blocker (Choi Y H, Lee M G. Xenobiotica. 2012; 42:483-95). Metformin was focused on because it can be modified easily by click chemistry using the cyanoguanidine condensation reaction (US Patent Application Publication 2012/0283299; Row et al., Biochem Biophys Res Commun. 2016; 469:783-9; Choi J, et al., Oncotarget. 2016). A strength of this approach is that metformin inhibits the Warburg effect, this inhibition being associated with activation of AMPK, an important biomarker for biguanide inhibition of cancer biomass assembly and activation of catabolic pathways (Faubert et al., Cell Metab. 2013; 17:113-24). A potential weakness of novel biguanides generated without a defined target is that they may fail to activate AMPK and may inhibit unrelated pathways, highlighting the structural importance of the moieties substituted at the N1 and N5 positions (Choi J, et al., Oncotarget. 2016). Another weakness is low potency of metformin for AMPK activation and the reason for this lack of potency remains an unanswered question (Berstein et al., Breast Cancer Res Treat. 2011; 128:109-17; Chandel et al., Cell Metab. 2016; 23:569-70), because a cognate protein target for metformin remains to be identified. It was reasoned that this problem could be potentially solved by testing the hypothesis that metformin physically interacts with CYP3A4 and inhibits epoxygenase activity. If so, we could then modify the metformin by click chemistry to better inhibit this target activity of CYP3A4.

In the present study, it was found that metformin is a weak inhibitor of CYP3A4 epoxygenase activity, exhibiting an IC₅₀ of 5 mM, similar to its IC₅₀ for cancer cell proliferation (Choi Y H, Lee M G. Xenobiotica. 2012; 42:483-95; Chandel et al., Cell Metab. 2016; 23:569-70). Metformin exhibited a type I spectroscopic spin shift in CYP3A4 incorporated in nanodiscs (Baas et al., Arch Biochem Biophys. 2004; 430:218-28), indicating binding at the substrate binding pocket. This result led to successful co-crystallization of metformin in the active site of CYP3A4, yielding a structure that enabled design of feasible “neo-biguanide” compounds, which could be used as probes for CYP3A4 epoxygenase activity in breast cancer. Screening was based not on AMPK activation, but rather on in silico docking and inhibition of CYP3A4 epoxygenase activity. This work led to the discovery of N1-hexyl-N5-benzyl-biguanide (HBB), a potent and selective inhibitor of CYP3A4 epoxygenase activity, which was subsequently found to exhibit strong activation of AMPK in breast cancer cells.

### Results

CYP3A4 Expression Correlates with ERα Expression in Breast Cancer

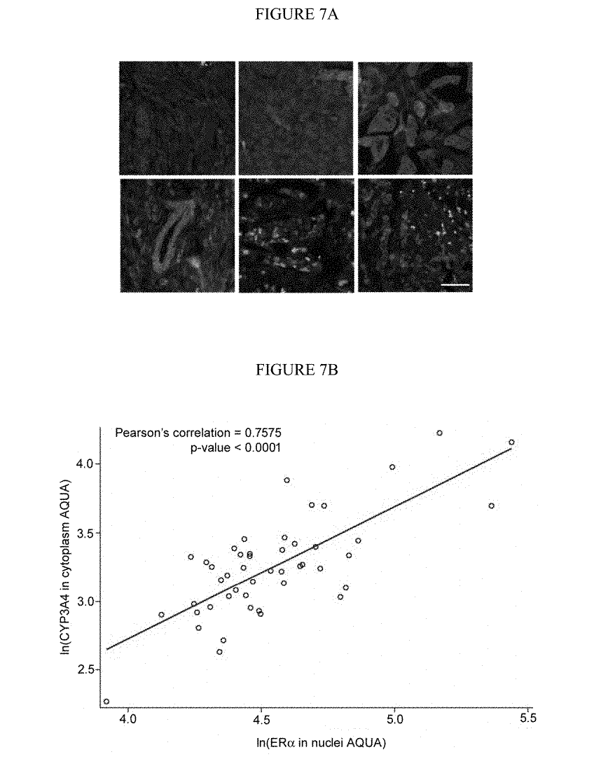

While CYP3A4 promotes the growth of ER+ breast cancer cells (Mitra et al., J Biol Chem. 2011; 286:17543-59) and has been associated with ER+ breast cancer (Murray et al., Histopathology. 2010; 57:202-11), it remains unknown whether CYP3A4 is expressed in ER+ breast cancer epithelia. Association of cytoplasmic CYP3A4 and nuclear ERα expression measured for breast tumor cores of unselected consecutive breast cancer patients (Kim et al., J Breast Cancer. 2012; 15:24-33; Bae et al., Am J Surg Pathol. 2012; 36:1817-25). TMA staining showed cytoplasmic localization of CYP3A4 (FIG. 7a). Cytoplasmic CYP3A4 was found to be associated with nuclear ERα, with a Pearson correlation coefficient of r=0.7575 (n=48; P-value <0.0001) (FIG. 7b).

EETs are Synthesized by CYP3A4 Nanodiscs

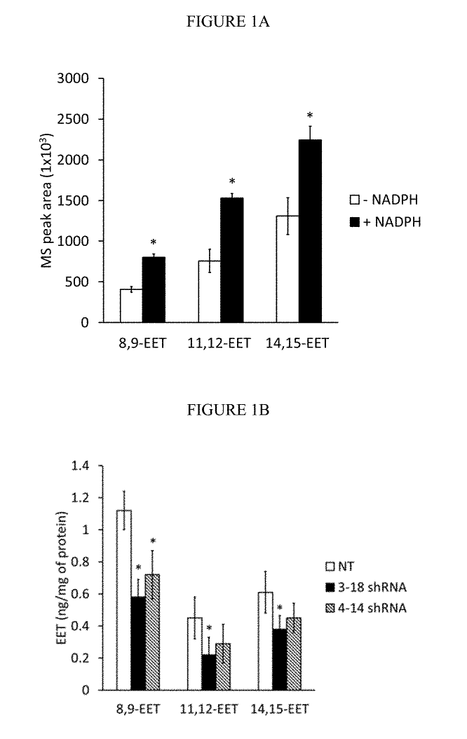

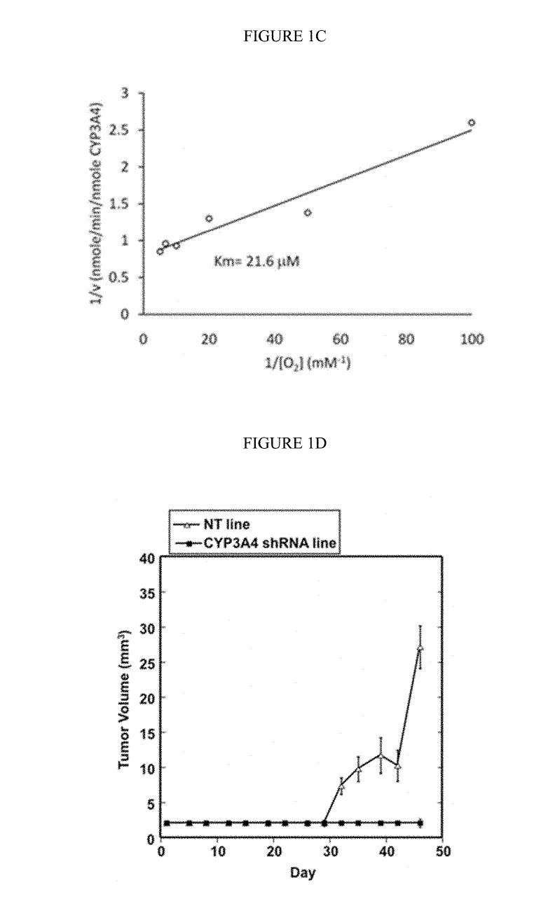

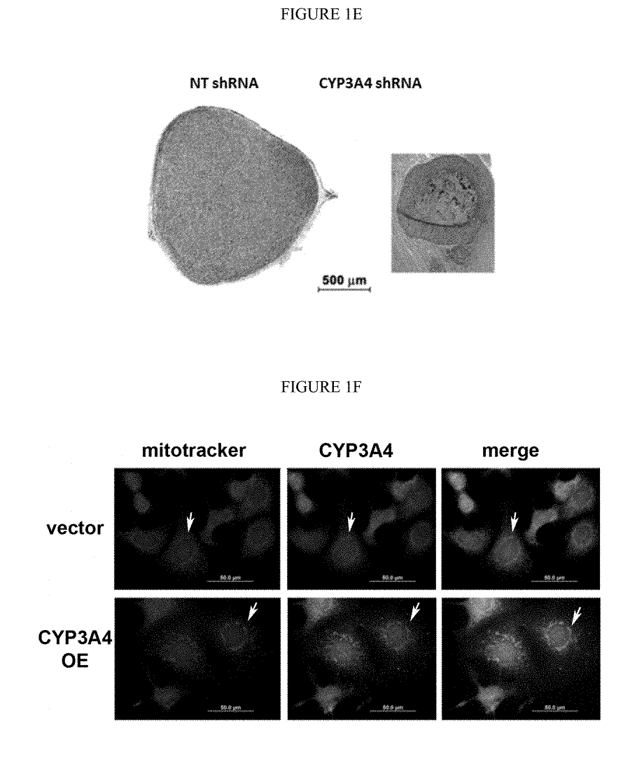

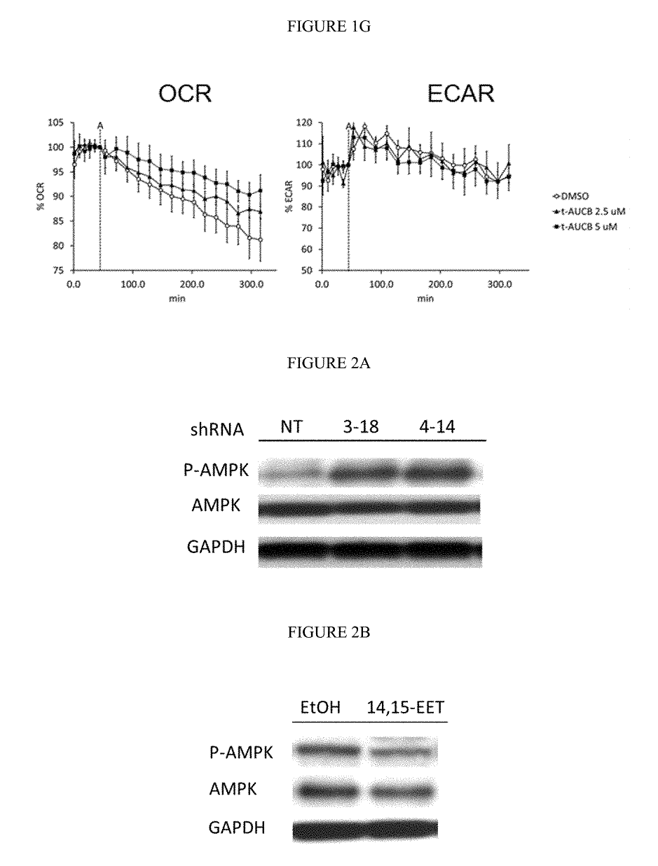

Whether CYP3A4 exhibits AA epoxygenase activity has been controversial. It was therefore asked whether nanodisc incorporated full-length native CYP3A4 synthesizes EETs (Grinkova et al., Biochem Biophys Res Commun. 2010; 398:194-8; Nath et al., J Biol Chem. 2007; 282:28309-20). CYP3A4 nanodiscs allow optical spectroscopy of heme-ligand interactions, permitting quantification of ligand-induced spin-shift of the Soret absorption band (Grinkova et al., Biochem Biophys Res Commun. 2010; 398:194-8; Nath et al., J Biol Chem. 2007; 282:28309-20; Denisov et al., J Biol Chem. 2007; 282:7066-76). CYP3A4 nanodiscs synthesized EETs in an NADPH-dependent fashion (FIG. 1a), confirming that CYP3A4 has significant AA epoxygenase activity, similar to CYP2C8 and CYP2J2 (Mitra et al., J Biol Chem. 2011; 286:17543-59). In addition, cellular EET levels in MCF-7 CYP3A4 knockdown cell lines 4-14 and 3-18 (Mitra et al., J Biol Chem. 2011; 286:17543-59) show a reduction of total EET levels (FIG. 1b). These results provide direct biochemical evidence that CYP3A4 synthesizes EETs and that sensitivity of CYP3A4 knockdown cells to exogenous EETs may be due, in part, to reduction of endogenous EETs (Mitra et al., J Biol Chem. 2011; 286:17543-59).

CYP3A4 Silencing is Associated with Delayed Escape from Tumor Dormancy

While vascular CYPs and exogenously supplied EETs have been implicated in escape of xenograft tumors from dormancy (Panigrahy et al., J Clin Invest. 2012; 122:178-91), the role of cancer cell-intrinsic CYPs has not been tested. The effect of CYP3A4 shRNA knockdown on tumor formation in the ER+HER2− MCF-7 orthotopic breast cancer model was therefore tested. The MCF-7 cell line exhibits CYP3A4 amplification (NCI-60 database; cBioPortal) (Cerami et al., Cancer Discov. 2012; 2:401-4) and is dependent on EETs for proliferation in culture (Mitra et al., J Biol Chem. 2011; 286:17543-59). Because EET biosynthesis is a novel activity for CYP3A4, it was first determined whether this enzyme activity occurs at oxygen concentrations characteristic of the tumor microenvironment (5-50 μM) (Ward J P. Biochim Biophys Acta. 2008; 1777:1-14; Kallinowski et al., Cancer Res. 1989; 49:3759-64). Using a continuous oxygen electrode, the O₂ Kₘ of CYP3A4 was 21.6 μM, which is within the range of intratumoral pO₂ (FIG. 1c). Tumor growth of the MCF-7 CYP3A4 knockdown cell line 3-18, chosen because it had the greatest CYP3A4 knockdown, was compared with a scrambled non-target shRNA cell line NT2 (Mitra et al., J Biol Chem. 2011; 286:17543-59). The NT2 tumors with scramble shRNA escaped from dormancy after day 30, whereas the 3-18 tumors weren't palpable even by day 45 (FIG. 1d), although nodules ≤1 mm were detected in each fat pad injected with 3-18 cells. CYP3A4 knockdown tumors exhibited central necrosis (5 of 6 evaluable mice), whereas the NT2 tumors did not (0 of 6 evaluable mice; FIG. 1e). This result means that the MCF-7 breast cancer model requires cancer cell-intrinsic CYP3A4 for escape from tumor dormancy.

CYP3A4 Co-Localizes with Mitochondria and Maintains Intracellular EET Levels

To determine the possible cancer cell-intrinsic roles of CYP3A4 in tumor growth, regulation of bioenergetics was focused on for two reasons. First, CYP enzymes can localize to mitochondria (Addya et al., J Cell Biol. 1997; 139:589-99) and CYP biosynthesis of EETs has been implicated in stabilization of mitochondrial function in cardiac myocytes (Katragadda et al., J Mol Cell Cardiol. 2009; 46:867-75). To test subcellular localization of CYP3A4, the full-length protein was stably over-expressed in the MCF-7 cell line C14 and compared with an MCF-7 empty vector control line, P7. Quantified by western blot, the C14 cell line exhibited >10-fold overexpression of CYP3A4 (C14 vs. P7 data not shown; t test P<0.05). Co-staining for CYP3A4 (fluorescein secondary antibody in green) and MitoTracker Red revealed intense co-localization of CYP3A4 and mitochondria in perinuclear regions more intensely in C14 as compared to P7, but co-localization was also observed in P7 (FIG. 1f). These results revealed that CYP3A4 localizes to breast cancer cell mitochondria.

EET Stabilization Promotes OCR, but not ECAR

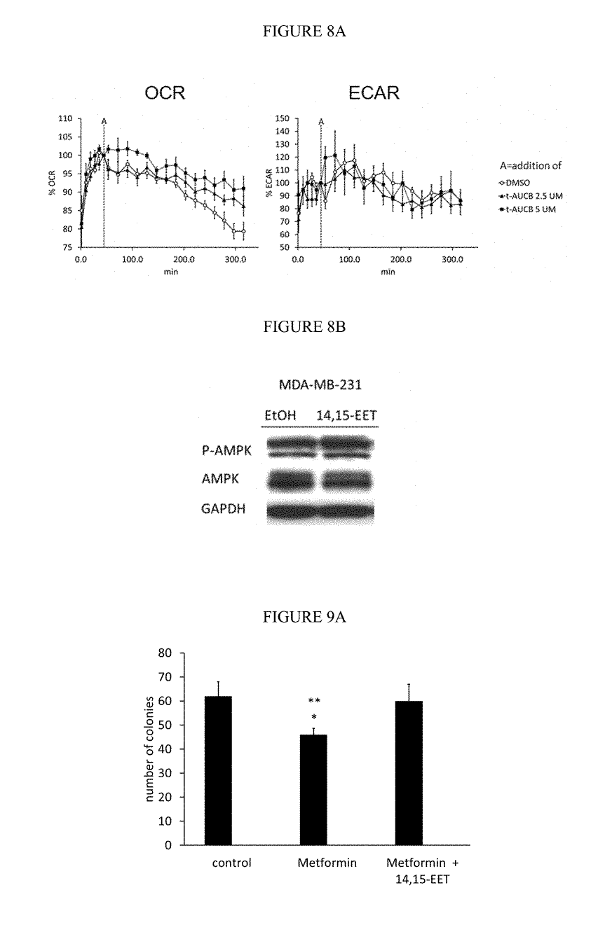

To investigate whether EETs influence mitochondrial function in breast cancer cells, it was tested whether stabilization of EETs affects mitochondrial respiration. Exogenous EETs failed to have immediately measurable effects on mitochondrial respiration (data not shown), perhaps because mitochondria are distant from the plasma membrane and exogenous EETs may have a capacity to esterify and traffic to membranes before reaching mitochondria. Inhibition of soluble epoxide hydrolase (sEH) is known to increase EET levels in cells and animals (Merabet et al., J Mol Cell Cardiol. 2012; 52:660-6). Therefore, a highly penetrant sEH inhibitor, t-AUCB (Hwang et al., Bioorg Med Chem Lett. 2006; 16:5773-7), was used to test whether stabilization of cellular EETs can modulate mitochondrial oxygen consumption. Treatment with t-AUCB increased the oxygen consumption rate (OCR) in MCF-7 cells within 15 minutes in a dose-dependent fashion and was sustained to the endpoint at 250 minutes (t test P values for OCR endpoints of P=0.0244 for 2.5 and 0.0033 for 5.0 μM t-AUCB) (FIG. 1g). Similar results were observed for the triple negative, CYP3A4-non-amplified, MDA-MB-231 cell line treated with t-AUCB (t test P values for OCR endpoints of P=0.023 for 2.5 and 0.00080 for 5.0 μM t-AUCB) (FIG. 8a). Notably, no effect of t-AUCB was observed on the extracellular acidification rate (ECAR) in either cell line (FIG. 1g), which in part reflects lack of change of lactate production rates. This result suggests that the primary effect of CYP epoxygenase activity is on OCR rather than ECAR.

CYP3A4 Silencing Activates and (±)-14,15-EET Inhibits AMPK

Phosphorylation of AMPK on Thr172 (pAMPKc) is an indirect measure of ATP stores and the ATP/AMP ratio in cells (Moore et al., Eur J Biochem. 1991; 199:691-7), while increase of pAMPK phosphorylation can reflect a shift to catabolism by which biomass is mobilized for energy production. While ATP levels reflect, in part, a composite of activity of glycolytic flux and oxidative phosphorylation, relative increases can also indicate biomass mobilization and energy stress. Based on previous studies of protection of cardiac myocytes by EETs (Katragadda et al., J Mol Cell Cardiol. 2009; 46:867-75), it was hypothesized that EETs derived from CYP epoxygenase activity may be involved in protection of OCR and membrane potential (ΔΨm), mitochondrial ATP production and suppression of pro-catabolic AMPK function. It was therefore asked whether CYP3A4 tonically suppresses pAMPKc by determining whether CYP3A4 knockdown induces pAMPK phosphorylation in breast cancer cells.

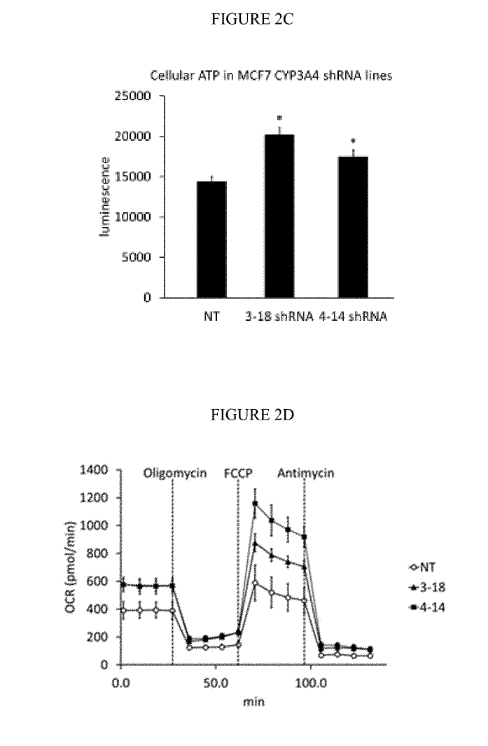

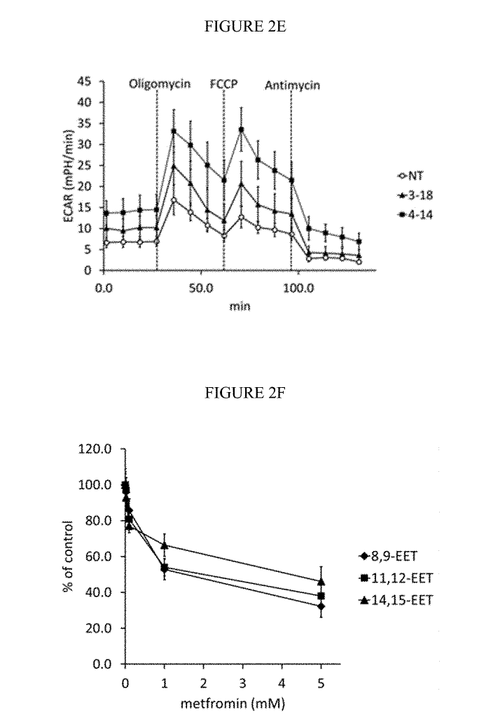

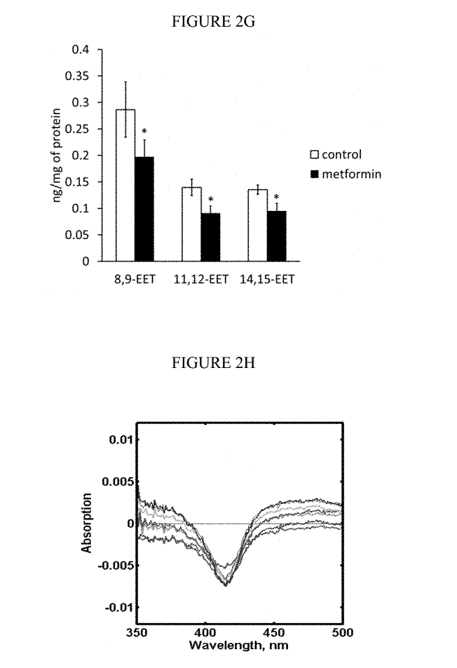

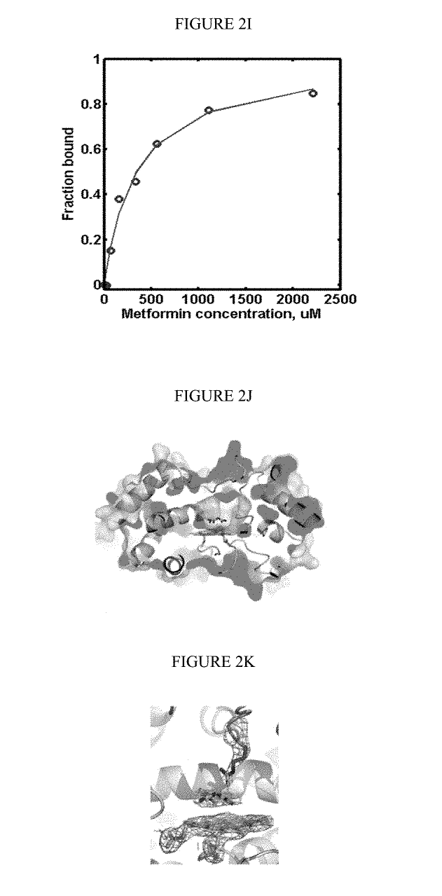

CYP3A4 silencing in the MCF-7 cell line was associated with steady state activation of pAMPK in two independently isolated CYP3A4 knockdown cell lines (FIG. 2a; 3-18 vs. NT2: P=0.0013; 4-14 vs. NT2: P=0.024) associated with 40 and 60% reduction of steady state CYP3A4 levels respectively (Mitra et al., J Biol Chem. 2011; 286:17543-59). Consistent with this finding, (±)-14,15-EET treatment of MCF-7 cells (24 hours) was associated with suppression of pAMPKc (FIG. 2b; P=0.023). In contrast, (±)-14,15-EET had no effect on AMPK phosphorylation in the triple negative breast cancer cell line MDA-MB-231 (FIG. 8b), suggesting that effects of exogenous EET are tumor cell type specific. Despite exhibiting decreased proliferation, mitogenesis, clonogenicity (Mitra et al., J Biol Chem. 2011; 286:17543-59) and tumorigenicity (FIG. 1d,e), CYP3A4 knockdown caused higher ATP levels (FIG. 2c), increased baseline OCR (P=8.2×10⁻⁵ and 0.00034) and ECAR (P=0.020 and P=0.00060) for the 3-18 and 4-14 cell lines (FIG. 2d,e; 100,000 cells per well), and increased spare respiratory capacity 1.5-fold and 3.0-fold. These findings are consistent with CYP3A4 suppression of catabolism and indicate that CYP3A4 knockdown results in compensatory increase in basal OCR and unexpectedly ECAR, suggesting increased lactate production. Thus, increase of steady state AMPK phosphorylation with CYP3A4 knockdown reflects energy stress and promotes increased ATP, while tumorigenicity is paradoxically abrogated. These findings strongly support a model in which CYP monooxygenases suppress AMPK activation through their EET products and under conditions of CYP knockdown catabolism compensates for loss of mitochondrial function, serving as an inefficient, albeit successful, means to maintain cellular ATP pools. There is also a latent respiratory capacity that can potentially be tapped.

Metformin Inhibits CYP3A4 and Reduces EETs in ER+ Breast Cancer Cells

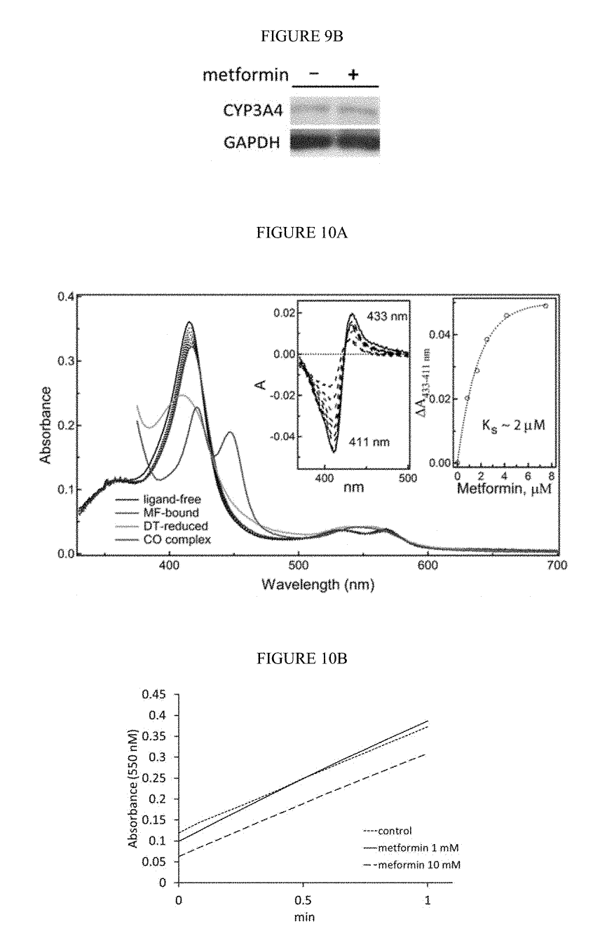

Metformin is a well-known pharmacological activator of AMPK in breast cancer cells (Zakikhani et al., Cancer Res. 2006; 66:10269-73) and has inhibitory activity against breast cancer in xenograft models (Liu et al., Cell Cycle. 2009; 8:2031-40; Iliopoulos et al., Cancer Res. 2011; 71:3196-201; Ma et al., BMC Cancer. 2014; 14:172). It was hypothesized that metformin may inhibit CYP3A4-mediated EET biosynthesis, leading to release of AMPK from EET-mediated inhibition, which would be a novel mechanism of action. It was therefore tested whether metformin affects CYP3A4-mediated EET biosynthesis and intracellular EET levels. Metformin inhibited NADPH-dependent microsomal CYP3A4 synthesis of the (±)-8,9, (±)-11,12, and (±)-14,15-EET regioisomers, exhibiting IC₅₀ values of 1.5, 2.2, and 4.5 mM, respectively (FIG. 2f). In contrast, the epoxygenase activity of CYP2J2, implicated in cancer progression (Jiang et al., Cancer Res. 2005; 65:4707-15; Jiang et al., Cancer Res. 2007; 67:6665-74), was not inhibited by 0.75, 1.5 or 3 mM metformin (data not shown). Furthermore, intracellular EET levels in MCF-7 cells were reduced by metformin (5 mM) after 6 hours of treatment (FIG. 2g). Concurrent exposure of MCF-7 cells to (±)-14,15-EET (1 μM) with metformin (1 mM) restored clonogenicity inhibited by metformin alone (FIG. 9a). Metformin did not affect the expression of CYP3A4 (FIG. 9b). Together, these results suggested not only a direct interaction between metformin and CYP3A4, but also suggested that activation of AMPK by metformin may, in part, be due to CYP inhibition.

Metformin Causes a Spectral Spin Shift on CYP3A4

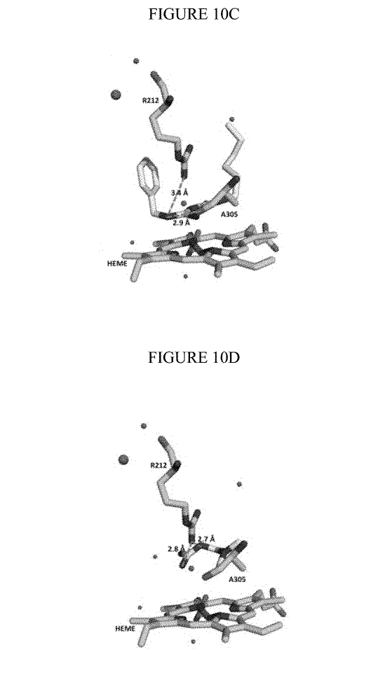

To determine whether there is a physical association between metformin and CYP3A4, CYP3A4 nanodiscs were used to measure a shift of the Soret band, which would provide spectral information on interaction of metformin with the CYP3A4 heme in the active site of the enzyme (Grinkova et al., Biochem Biophys Res Commun. 2010; 398:194-8; Nath et al., J Biol Chem. 2007; 282:28309-20; Denisov et al., J Biol Chem. 2007; 282:7066-76). Interaction of metformin with CYP3A4 nanodiscs resulted in a type I spin shift trough at 415 nm (FIG. 2h) and a spectral dissociation constant of Kₛ=400 μM (FIG. 2i). The type I spectral change was observed at high metformin concentrations, which may mean that more than one metformin molecule can potentially pack in the active site and partially displace water coordinating to the heme iron. When metformin was incubated with a soluble, truncated form of CYP3A4 (Δ3-22) lacking the hydrophobic N-terminal domain, a type II spin shift was observed with a peak at 433 nm and a spectral dissociation constant of Kₛ=2 μM (FIG. 10a). The type II spin shift observed with CYP3A4 (Δ3-22) may mean that metformin can either coordinate to the heme iron or somehow change the water molecule coordination to the heme iron in the soluble N-terminal truncated CYP3A4 protein. It was also confirmed that metformin does not inhibit the transfer of electrons from CPR by measuring the rate of reduction of cytochrome c by CPR, which was not affected by metformin (FIG. 10b), arguing against interference of CYP-CPR interaction by metformin. These results suggested that metformin activity in the MCF-7 cell line is directly related to binding of the CYP3A4 heme.

Metformin Co-Crystalizes in the CYP3A4 Active Site

Metformin was successfully co-crystallized with a soluble, truncated form of CYP3A4 (Δ3-22) enabling the X-ray structure to be solved to a resolution of 2.6 Å (FIG. 2j, Table 1). Metformin is positioned over the heme and is also close enough to R212 in the F-F′ loop for H-bonding interactions. This co-crystal illustrates the flexibility of the CYP3A4 active site tertiary structure, particularly in the F-F′ loop, where the distance from the distal nitrogen of R212 to the heme iron is 18.5 Å in the ritonavir co-crystal (3XNU.pdb) compared to 6.1 Å in the metformin co-crystal (5G5J.pdb). It is unexpected that R212 can approach within H-bonding distance of metformin since both groups are strong bases and both should be positively charged at physiological pH. However, the electron density is consistent with metformin being non-planar. The adoption of greater sp3 character could possibly lower the pKₐ of metformin, especially in the confines of the active site near the heme iron, thereby enabling R212 to H-bond with metformin (FIG. 2k) (FIG. 2l; stereo view). The amino acid residues of CYP3A4 in closest proximity to metformin were R212, A305, S119, R105, and A370.

N1-Hexyl-N5-Benzyl-Biguanide (HBB) Tightly Binds the CYP3A4 Active Site

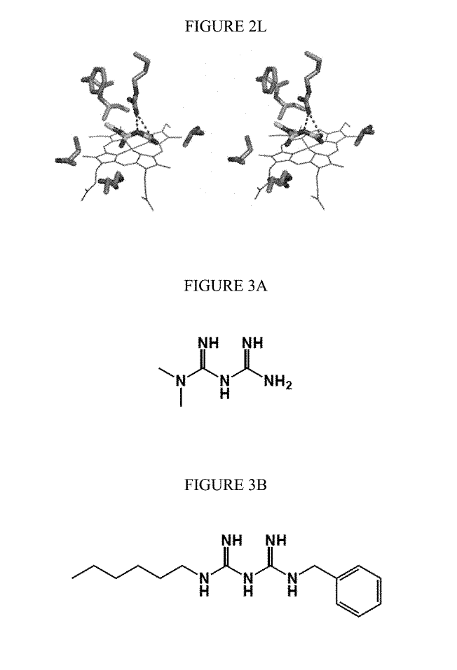

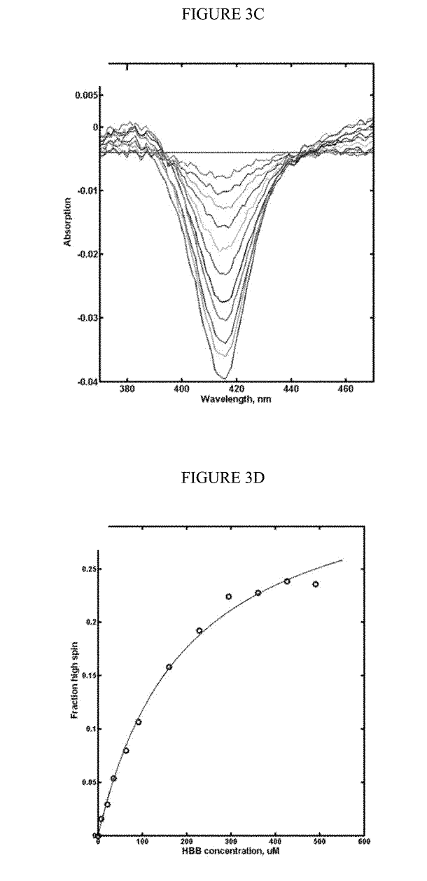

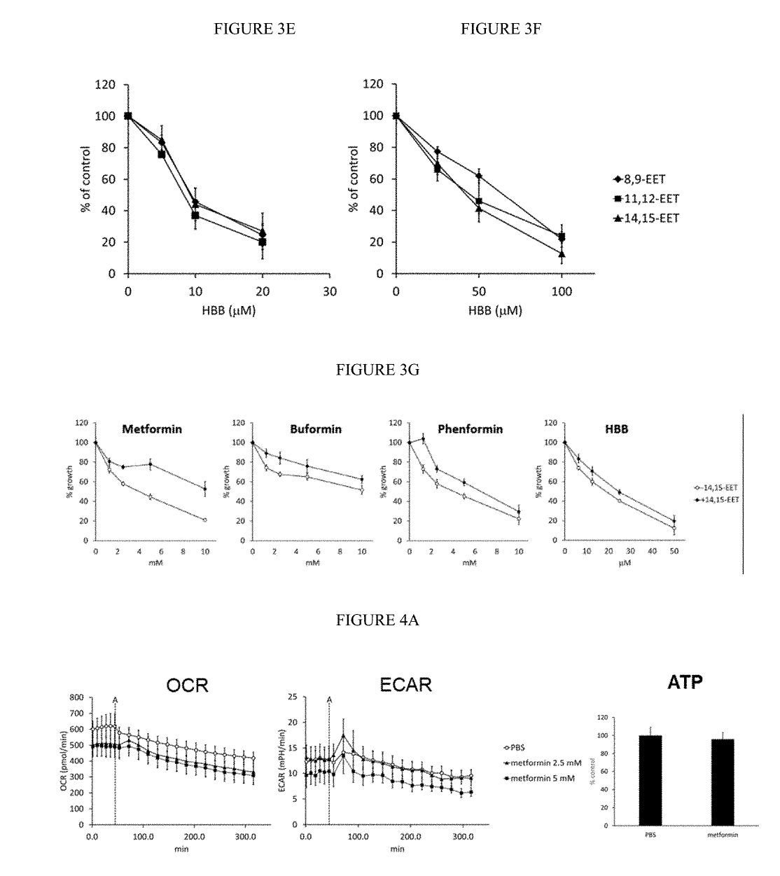

The CYP3A4-metformin crystal structure was next used to reverse-engineer biguanides expected by structure-based design to be more potent inhibitors of CYP3A4 epoxygenase activity. Biguanides allow combinatorial synthesis of highly diverse chemical entities with a wide range of chemical and pharmaceutical properties (US Patent Application Publication 2012/0283299). It was hypothesized that novel biguanides (neo-biguanides) with higher dock scores than metformin (FIG. 3a), indicating tighter binding could be discovered by in silico docking (Jain A N. J Comput Aided Mol Des. 2007; 21:281-306). The CYP3A4-metformin co-crystal structure was used to dock 18 other biguanides in the active site of CYP3A4 (Table 2). Docking scores were compared using the Surflex Dock program (Jain A N. J Comput Aided Mol Des. 2007; 21:281-306), with a higher dock score indicating a more favorable docking interaction. Of the 5 compounds with the highest dock scores, syntheses of compounds 2, 4, and 5 were most feasible and therefore performed.

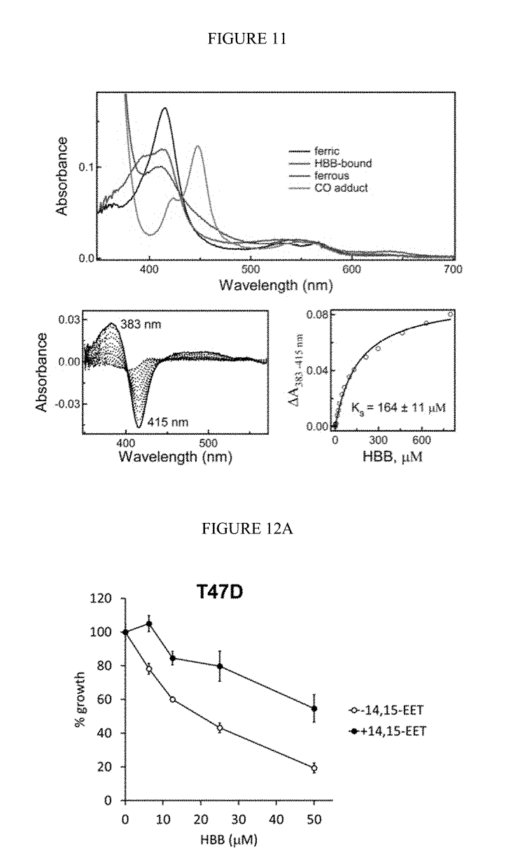

The neo-biguanide compound 4 [N1-hexyl-N5-benzyl-biguanide; HBB] (FIG. 3b) was chosen for further study because it was the most potent inhibitor of the MCF-7 breast cancer cell line (IC₅₀=20 μM) when compared to neo-biguanide compounds 2 [N1-hexyl-N5-[(1H-imidazol-2-yl)methyl]-biguanide; HIB] (IC₅₀˜500 μM and 5 [N1-hexyl-N5-(pyridin-4-ylmethyl) biguanide; HPB] (IC₅₀=40 μM) (Table 3). The neo-biguanide compounds were more potent inhibitors of cell growth than metformin, buformin, or phenformin (Table 3). HBB potently inhibited ER+ and triple negative breast cancer cell lines (Table 4). Using in silico modeling, HBB docked in the CYP3A4-metformin co-crystal structure (FIG. 10c) with a >2 log higher dock score (score=7.11) than the majority of biguanides tested (Table 2). The most energetically favorable docked pose of HBB exhibited proximity to the heme (FIG. 10c vs. 10d). Unlike metformin, a co-crystal of CYP3A4 and HBB could not be obtained despite co-crystallization experiments performed under a range of conditions (Sevrioukova and Poulos; unpublished data). Nonetheless, HBB exhibited a strong type I spin shift with CYP3A4 nanodiscs (Kₛ=110 μM) (FIG. 3c,d) and soluble CYP3A4 Δ3-22 (Kₛ=164 μM) (FIG. 11). Spin shift results support a close proximity of HBB and the heme iron of CYP3A4.

HBB Selectively Inhibits CYP3A4 Epoxygenase Activity

HBB selectively inhibited microsomal CYP3A4 epoxygenase activity. The IC₅₀ values for inhibition of CYP3A4 synthesis of (±)-8,9, (±)-11,12, and (±)-14,15-EET were 9.5, 8.0, and 9.5 μM (FIG. 3e). The IC₅₀ values for CYP2C8 for these same regioisomers were 65, 50, and 45 μM (FIG. 3f). This 5-to-7-fold greater selectivity of HBB for CYP3A4 epoxygenase activity indicates that HBB can be used as a selective chemical probe for CYP3A4 epoxygenase activity in cancer cell growth and signaling studies, although it is recognized that other CYPs may also be inhibited.

Biguanide Inhibition of Breast Cancer Cell Growth is Partly Rescued by (±)-14,15-EET

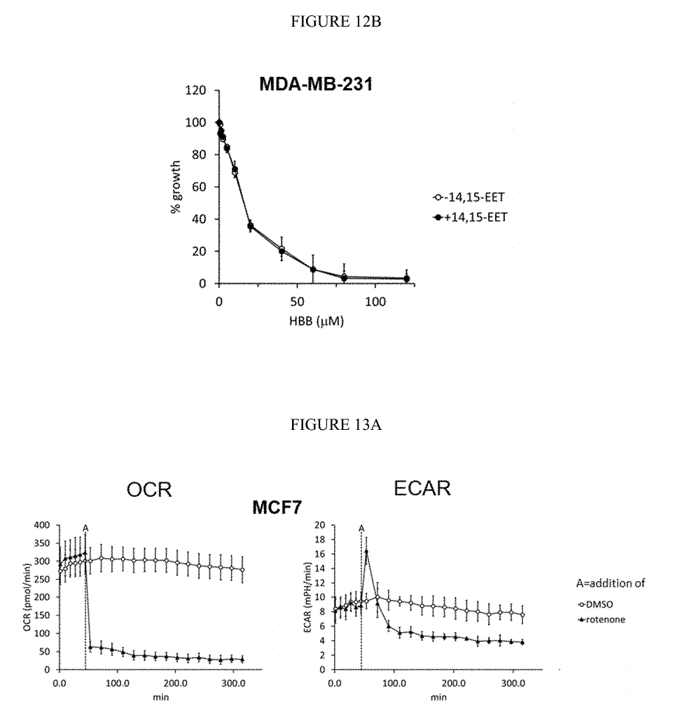

Whether metformin and related biguanides inhibit the breast cancer cell lines, in part, through inhibition of (±)-14,15-EET biosynthesis, was next sought to be determined. For the MCF-7 cell line, (±)-14,15-EET abrogated the most of metformin growth inhibition and part of buformin, phenformin and HBB inhibition (FIG. 3g). While (±)-14,15-EET partially abrogated HBB-mediated growth inhibition of the MCF-7 cell line, it abrogated most of the inhibition of the T47D cell line (FIG. 12a). These results suggest variable (±)-EET sensitivity of different cell lines, but a common dependence on this metabolite. Notably, the MDA-MB-231 cell line exhibited no dependence on (±)-14,15-EET for growth, suggesting HBB may be inhibiting different metabolite growth requirements or targeting different pathways (FIG. 12b).

HBB Causes Immediate OCR Inhibition and Transient ECAR Activation

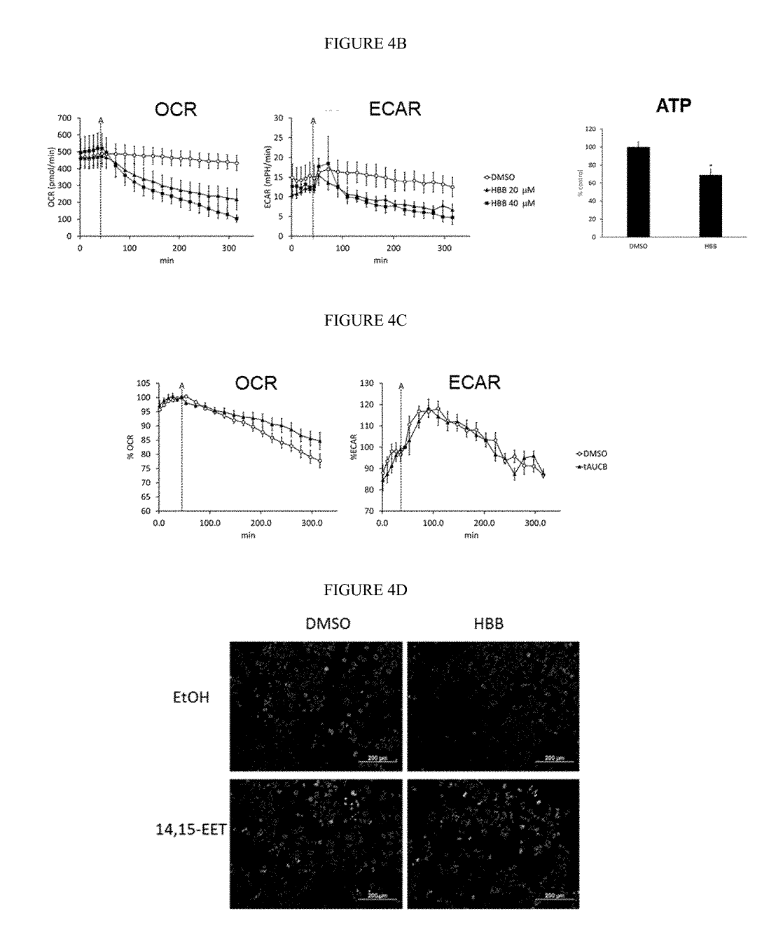

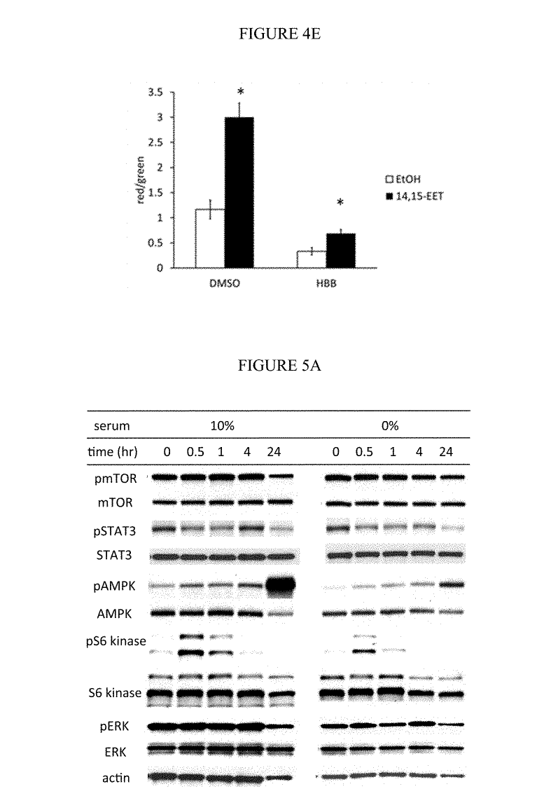

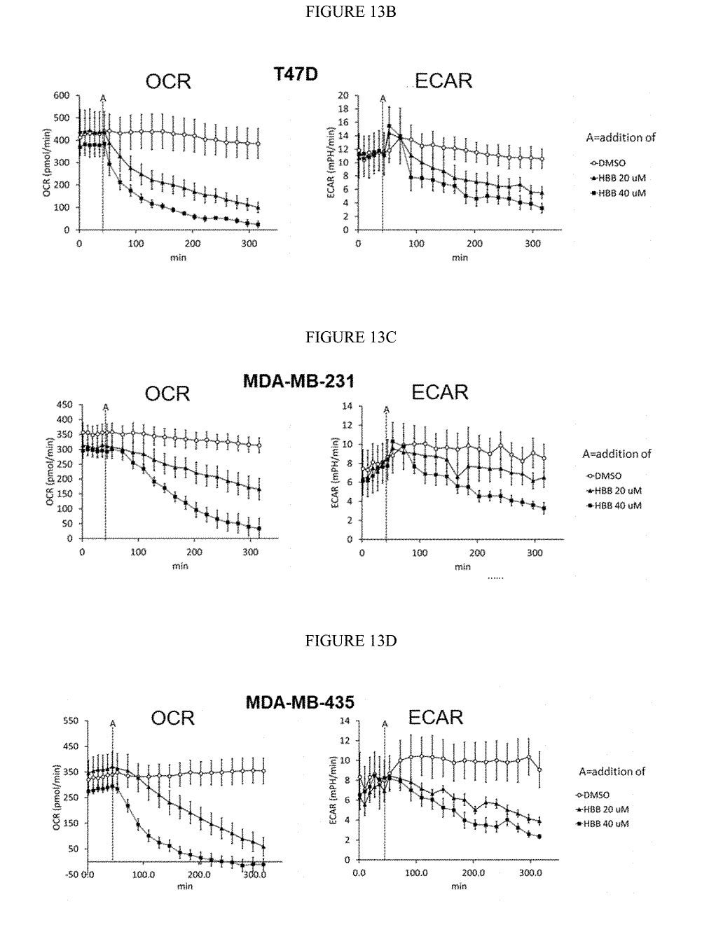

Biguanide inhibition of breast cancer has been proposed to inhibit mitochondrial oxygen consumption, in part, through inhibition of complex I (El-Mir et al., J Biol Chem. 2000; 275:223-8; Wheaton et al., Elife. 2014; 3:e02242). To compare the effects of metformin and HBB on OCR and ECAR on breast cancer cell lines, an extracellular flux analyzer was used to measure OCR and ECAR. Metformin (2.5 or 5 mM) treatment of the MCF-7 cell line resulted in marginal reduction of OCR beginning at ˜30 minutes, which was not statistically significant (t test), and a transient ECAR spike centered at 15 minutes followed by recovery of ECAR (FIG. 4a; left panel OCR, middle panel ECAR). The effect of metformin on ECAR was also marginal and failed to deplete cellular ATP at 6 hours (FIG. 4a; right panel, 5 mM). HBB treatment (20 and 40 μM) resulted in more rapid and dose-dependent reduction of OCR within 15 minutes, indicating that HBB is a more than 250-fold more potent inhibitor of OCR than metformin (FIG. 4b; left panel; endpoint HBB 20 μM P=0.0014, by t test; HBB 40 μM P=2.5×10⁵ and the pairwise differences were also significant: 20 μM vs. DMSO P=0.00389; 40 μM vs. DMSO P=0.00755; 20 vs. 40 μM P=0.00366). HBB also induced a transient spike of ECAR, lasting more than 15 minutes, followed by significant inhibition of ECAR without recovery (FIG. 4b; middle panel; HBB 20 μM P=0.0021 and HBB 40 μM P=0.00050, by t test and the pairwise differences were also significant 20 μM vs. DMSO P=0.00384; 40 μM vs. DMSO P=0.00526; 20 vs. 40 μM P=0.00142). HBB depleted ATP by 30% at 6 hours (P<0.05) (FIG. 4b; right panel), indicating that in contrast to CYP3A4 knockdown, acute inhibition depletes ATP stores. The potent complex I inhibitor rotenone (1.0 μM) served as a positive control to demonstrate that the OCR and ECAR inhibition of HBB (20-40 μM) is consistent with rapid inhibition of the ETC (FIG. 13a; left panel OCR, right panel ECAR; P<0.0001 by curve fitting for both), in contrast to weak inhibition by metformin even at high concentrations (5 mM) (FIG. 4a).

To determine whether suppression of OCR followed by ECAR is observed with other breast cancer cell lines, the T47D, MDA-MB-231 and MDA-MB-435/LCC6 cell lines were treated with HBB and subject to extracellular flux analysis (FIG. 13b,c,d left panels OCR, right panels ECAR). Similar dose-dependent signatures were observed and they were significant for all comparisons of DMSO vs. 20 and 40 μM and 20 vs. 40 μM HBB (OCR and ECAR endpoints; HBB vs. DMSO control P all <0.015). Notably, the MDA-MB-231 cell line exhibited the least inhibition of ECAR, despite OCR sensitivity that was comparable to the other cell lines.

EET Stabilization Promotes Resistance to HBB-Mediated OCR Inhibition

To determine whether the HBB effect can be opposed by EET stabilization with epoxide hydrolase inhibition, t-AUCB (5 μM) was added 120 minutes before addition of HBB (5 μM). The addition of t-AUCB resulted in a 1.5-fold reduction in the rate of OCR decline, but had no effect on ECAR (FIG. 4c; OCR left panel, ECAR right panel). This experiment strongly suggests that HBB inhibition of OCR is, in part, due to HBB inhibition of epoxygenase activity in breast cancer cells. This experiment also suggests that the primary effect of EETs is on stabilization of OCR, not ECAR.

HBB Inhibits ΔΨm, in Part, Through Suppression of CYP Epoxygenase Activity

It was then asked whether EETs protect the ETC in the presence of HBB. Using the indicator dye JC-1 to measure ΔΨm, it was tested whether HBB inhibits ΔΨm, in part, through depletion of EETs, which have been reported to stabilize ΔΨm in cardiac myocytes (Katragadda et al., J Mol Cell Cardiol. 2009; 46:867-75; Batchu et al., Can J Physiol Pharmacol. 2012; 90:811-23). With HBB treatment (20 μM for 4 hours for the MCF-7 and MDA-MB-231 cell lines), JC-1 dye exhibited a rapid shift in mitochondrial fluorescence from red to green, indicating reduction of ΔΨm (FIGS. 4d,e MCF-7; MDA-MB-231). This reduction of ΔΨm was partly rescued in both the MCF-7 and MDA-MB-231 lines by pre-treatment with exogenous (±)-14,15-EET (P<0.05 for each) (FIG. 4d,e). These results indicate that ΔΨm depends, in part, on CYP epoxygenase activity and EETs, consistent with our observation of the dependence of OCR on EETs.

HBB Rapidly Activated pAMPK and Inhibited Stat3, mTOR and ERK

Previous studies of breast cancer cells treated with high concentrations of metformin (10-30 mM) and extended treatment times (48 hours) showed activation of AMPK phosphorylation and reduced Stat3 and mTOR phosphorylation (Zakikhani et al., Cancer Res. 2006; 66:10269-73; Deng et al., Cell Cycle. 2012; 11:367-76). Similar delay was observed of AMPK activation with metformin (5 mM), first detectable after 6 hours (data not shown). In contrast, HBB activated AMPK at 20 μM dosing within 30 minutes to 1 hour, independent of serum (FIG. 5a-c). In contrast, Stat3 activation is partially serum dependent and known to be rapidly activated by EETs in breast cancer cells (Mitra et al., J Biol Chem. 2011; 286:17543-59); in agreement with this observation, HBB inhibited early Stat3 phosphorylation more effectively in the presence of serum suggesting inhibition of immediate early growth factor signaling pathways active under this condition (FIG. 5a-c). HBB treatment also resulted in rapid and transient activation of S6 kinase at 30 minutes (FIG. 5a-c), which was independent of serum starvation; this finding shows a novel, stress-related S6 kinase activation independent of mTOR S2448 phosphorylation. Significant inhibition of mTOR, S6 kinase and ERK occurred by 24 hours in the presence or absence of serum (FIG. 5a-c). While HBB effects on signaling were qualitatively similar to metformin, they were immediate early effects and at 250-fold lower dosing.

HBB Inhibits MCF-7 Xenograft Tumor Growth

HBB was tested for tumor inhibition in orthotopic mammary fat pad models. Dosing of HBB begun the day after implantation and 4 mg/kg/day ip was found to be a minimal effective dose (MED) in the MCF-7 model (FIG. 15a). MCF-7 tumors in mice treated with HBB showed slower growth than PBS treated control mice (Gompertzian fitting; P=0.0354). End point tumor weights of HBB treated mice were 34% lower than control (mean±SEM: 335±55 mg in PBS group, n=20 vs. 220±35 mg in the HBB group, n=19; P=0.088). At the same HBB dosing, the MDA-MB-231 xenograft tumors exhibited no inhibition (FIG. 15b). Weight loss was less than 8% in both models (FIG. 9c,d).

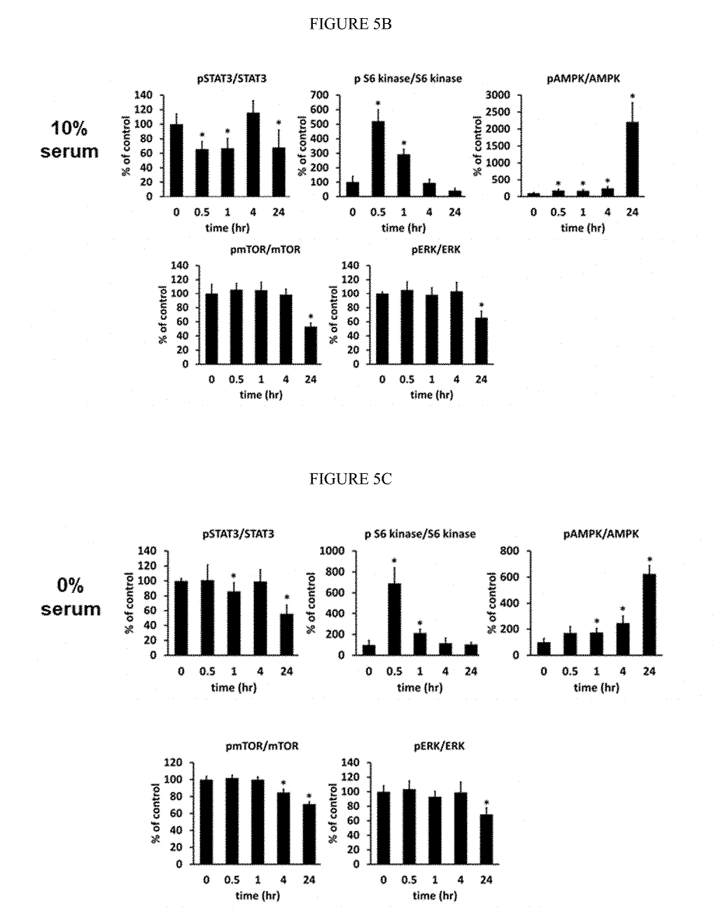

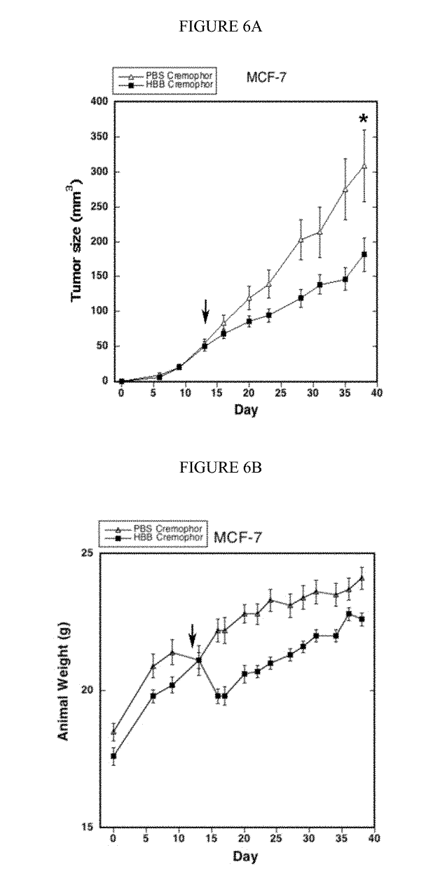

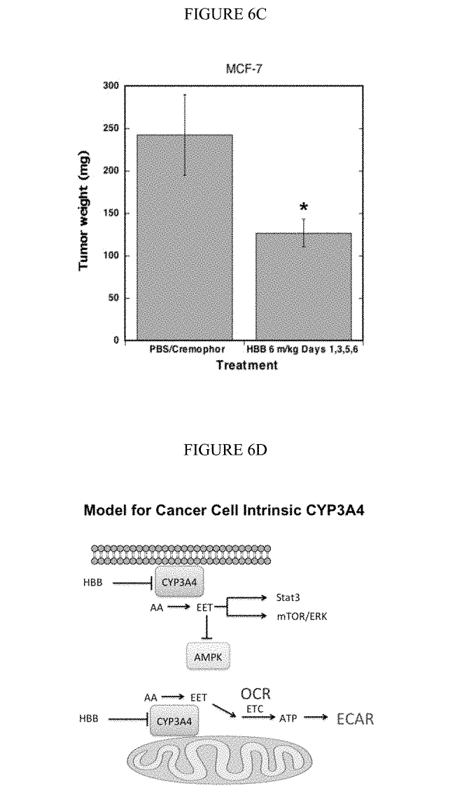

Dose escalation of HBB to 6 mg/kg/day was then performed in the MCF-7 model and dosing began once the tumors reached a mean size of 50 mm³. Early activity (FIG. 6a) was found, but significant, reversible weight loss (FIG. 6b) after three consecutive daily doses at 6 mg/kg (following arrow). Dosing was changed to 4 of 7 days with no more than two consecutive days of dosing (24 mg/kg/week ip). This dosing schedule resulted in a significant reduction of tumor growth (P=0.039) (FIG. 6a) and by tumor weights at the end point, which were 48% lower in the HBB group [242±48 mg for the PBS group (n=15) vs. 127±16 mg for the HBB group (n=13) (mean±SEM); (P=0.034)] (FIG. 6c). Weight loss was at maximum 11% and <6% by the end of the study (FIG. 6b). There was no detectable HBB related hepatic or cardiac toxicity and no difference of non-fasting blood glucose levels. HBB therefore exhibits significant single agent activity vs. the MCF-7 model with the toxicity of reversible weight loss.

Correlative Biomarkers of HBB Efficacy

Reverse phase protein microarray (RPPA) analysis of 155 signaling proteins, after correction for multiple comparisons, revealed two highly suppressed proteins in MCF-7 but not MDA-MB-231 tumors treated with HBB: mTOR level (P=4.06×10⁻⁶) and PKC-ζ/λ phospho-threonine 410/403 (P=0.000119) (Table 5a,b). Suppression of these proteins is consistent with suppression of pathways involved in bioenergetics and both are associated with chemotherapy resistance (Rimessi et al., Cell Cycle. 2012; 11:1040-8; Mondesire et al., Clin Cancer Res. 2004; 10:7031-42).

Model for HBB Inhibition of CYP3A4 Epoxygenase Activity

It is proposed that HBB inhibition of ER+ tumor growth is due, in part, to direct inhibition of membrane associated non-mitochondrial CYP3A4, thereby suppressing Stat3 and mTOR signaling due to suppression of EET biosynthesis, resulting in AMPK activation (FIG. 6d, membrane diagram). This model is supported by the finding that Stat3 knockdown inhibits EET-driven cell proliferation (Mitra et al., J Biol Chem. 2011; 286:17543-59). HBB also inhibits CYP3A4 at the mitochondria, resulting in immediate inhibition of respiration (FIG. 6d, mitochondrial diagram). In this model, EETs inhibit AMPK through unknown mechanisms. Testing the model by RPPA analysis of MCF-7 cells treated with (±)-14,15-EET, Stat3 phosphorylation is activated on Y705 and S727 at 15 minutes (t test P=1.4×10⁻⁵ and P=0.00065), as is mTOR phosphorylation on S2448, while the total mTOR level is also increased (t test P=0.011 and P=0.0077). These results suggest that EETs regulate mTOR levels in addition to phosphorylation. This model therefore proposes two sites of CYP3A4 activity, at cellular membranes and at mitochondria.

### Discussion

While cytochrome P450 enzymes have been well studied in terms of cancer drug metabolism, less is known about their cell autonomous functions in cancer epithelia and contribution to “metabolic reprogramming” that promotes cancer progression. In contrast to prior studies of EETs and their roles in tumor angiogenesis (Panigrahy et al., J Clin Invest. 2012; 122:178-91; Zhang et al., Proc Natl Acad Sci USA. 2013; 110:6530-5), as described herein it has been validated that breast cancer cell intrinsic CYP3A4 as required for ER+ tumor growth and as a target of metformin and HBB, which inhibit the biosynthesis of EETs on which the MCF-7 breast cancer cell line depends for proliferation, mitogenesis and clonogenicity (Mitra et al., J Biol Chem. 2011; 286:17543-59). The presence of CYP3A4 in mitochondria, the stabilization of the ETC by soluble epoxide hydrolase inhibition and the EET-mediated partial rescue of breast cancer cells from biguanide inhibition suggests that CYP epoxygenase enzymes promote the function of breast cancer mitochondria. CYP3A4 appears to function as a suppressor of catabolism, mediated through its EET products and their effects on cancer cell mitochondria.

The sensitivity of breast cancer cell lines to neo-biguanide disruption of oxygen consumption appears to be widespread and sensitivity of the MCF-7 cell line to EET rescue from HBB in vitro is correlated with sensitivity to HBB in vivo. Nonetheless, rescue of the ETC by EETs does not translate into rescue of cell proliferation in the case of the mutant K-Ras driven triple negative cell line MDA-MB-231. Correlating with these results, AMPK wasn't suppressed by EETs in the MDA-MB-231 cell line nor were these cells rescued from HBB by EETs, suggesting that oncogenic signaling pathways can override EET effects. While in the current body of work EET responsiveness in vitro is associated with biguanide sensitivity in vivo, further studies will be needed to define the spectrum of breast cancer sensitive to biguanides. To aid in this process, there are now tools in hand to better characterize breast cancer cells in terms of the impact of EETs on bioenergetics, specifically whether EET rescues cells from HBB inhibition and whether EET suppresses AMPK. These tools may help predict in vivo sensitivity of tumors to neo-biguanides. Potency of neo-biguanides is also likely to be important because, even at 100-fold higher dosing than HBB, metformin has little activity on the MCF-7 xenograft (Ma et al., BMC Cancer. 2014; 14:172). A limitation of HBB is that it doesn't lower total EET levels in cancer cells, perhaps related to greater selectivity for CYP3A4 leaving other CYPs such as less sensitive CYP2C8 open for EET biosynthesis. Nonetheless, HBB potently disrupts EET signaling and metabolic pathways and gives a much-needed tool to develop neo-biguanides as chemical probes and candidate therapeutic agents.

There have previously been two mitochondrial targets suggested for metformin, complex I (El-Mir et al., J Biol Chem. 2000; 275:223-8; Wheaton et al., Elife. 2014; 3:e02242) and mitochondrial glycerophosphate dehydrogenase (mGPDH) (Madiraju et al., Nature. 2014; 510:542-6). The first mechanism discovered for metformin inhibition of the ETC at high concentrations (5 mM) involved inhibition of complex 1 (El-Mir et al., J Biol Chem. 2000; 275:223-8), as supported by experiments in which NADH dehydrogenase from S. cerevisiae was substituted into mammalian cells (Wheaton et al., Elife. 2014; 3:e02242). More recently, new evidence that metformin at much lower concentrations (50 μM) suppresses gluconeogenesis through inhibition of mitochondrial glycerophosphate dehydrogenase (mGPDH) (Madiraju et al., Nature. 2014; 510:542-6). This effect in turn inhibits the conversion of glycerol-3-phosphate to dihydroxyacetone phosphate by mGPDH with a concomitant decrease in donations of electrons directly to the ETC by co-enzyme Q (Baur J A, Cell Metab. 2014; 20:197-9). A third potential mechanism is now presented, supported by direct targeting by biguanides, direct stabilization of the ETC by soluble epoxide hydrolase inhibition and partial rescue by EETs in the presence of biguanides. If EETs play a general role in modulating ΔΨm and the ETC, perhaps at the level of the mitochondrial inner membrane, they could potentially impact the flux of electrons from complex I and the flux of electrons into the ETC from mGPDH function. Further studies will require analysis of the direct effects of EETs and biguanides on complex I and mGPDH activities.

The results described herein suggest that EETs are active in at least two locations in the cancer cell, the membrane compartment where Stat3/mTOR is activated and AMPK signaling is suppressed and mitochondria where OCR is promoted (FIG. 6d). Because EET stimulation of cell proliferation, mitogenesis and clonogenicity depends on Stat3 (Mitra et al., J Biol Chem. 2011; 286:17543-59), CYP3A4 and other CYPs may provide a mechanism that suppresses catabolism, permitting coupling of Stat3 mediated anabolism with mitochondrial function. Metformin may function by mass action to shut down bulk EET biosynthesis in the cell, whereas HBB may act more selectively at the mitochondria, where it has a potent and strategic effect inhibiting ΔΨm/OCR. Hence, exogenous EETs more easily abrogate the effects of metformin, as compared to HBB.

Emergence of CYP3A4 as a breast cancer target associated with mitochondria is likely to be important conceptually for development of novel therapeutics, as suggested by the newly discovered role of CYP3A4 as a “brake” on cellular catabolism in ER+ breast cancer. Clinical development of CYP3A4 as a therapeutic target will also require more detailed analysis of the balance between collective impact of CYP epoxygenase enzymes and their soluble epoxide hydrolase counterparts on cellular metabolism programs in cancer cells and their microenvironment. Neo-biguanides directed to ETC disruption alone and in combination with inhibitors of pathways synthetic lethal with CYP inhibition point toward new approaches for therapeutic development.

### Materials and Methods

Cell lines, Chemicals and Reagents

The MCF-7, MDA-MB-231 cell lines were a gift of Dr. Harikrishna Nakshatri (Indiana University) and short tandem repeat profiling (STR) verified by his laboratory. For xenograft experiments testing HBB activity, tumorigenic, estradiol responsive MCF-7 cells that were a gift from Dr. Deepali Saachdev (University of Minnesota) were used and STR verified by her laboratory. Their profile was similar to MCF-7 cells in ATCC, DSMZ, or JCRB databases. T47D cells were purchased from the American Type Culture Collection (ATCC; Manassas, Va.). MDA-MB-435 (LCC6) cells were obtained from Dr. Deepali Saachdev (University of Minnesota) and were a gift of Dr. Robert Clarke (Georgetown U.) and confirmed by STR. The MCF-7 cells used in xenograft studies were tested and found to be free of mycoplasma (Lonza). The MDA-MB-231 cells used for in vitro and xenograft studies were tested for pathogens by the RADIL Reference Laboratory (University of Missouri) (now IDEXX BioResearch) laboratory and were found to be free of viral pathogens and mycoplasma.

DMEM was purchased from GIBCO/Invitrogen (Carlsbad, Calif.). Charcoal- and dextran-stripped serum was purchased from Hyclone (Logan, Utah). FBS was assayed by the Potter laboratory and found to exhibit EET levels of <100 nM. EETs were provided purified by J. Capdevila (Vanderbilt University), J. Falck (University of Texas Southwestern) or purchased from Biomol International (Plymouth Meeting, Pa.) or Cayman Chemical Co. (Ann Arbor, Mich.). Phenomenex Luna C18 (250×4.6 mm, 5-μm particle size) columns were purchased from Phenomenex (Torrance, Calif.). Insect cell microsomes expressing recombinant human P450 CYP3A4 and CYP2C8 (Supersomes™) were purchased from Corning (Corning, N.Y.). JC-1 mitochondrial membrane potential probe was purchased from Thermo Fisher Scientific (Waltham, Mass.). XF Assay Kit and Mito Stress Kit, both 24 well, were purchased from Seahorse Bioscience (North Billerica, Mass.). Eicosanoid mass spectrometry standards (±)-8,9-(5Z,11Z,14Z)-EET, (±)-11,12-(5Z,8Z,14Z)-EET, and (±)-14,15-(5Z,8Z,11Z)-EET were purchased from Biomol International (Plymouth Meeting, Pa.). Arachidonic acid and d8-EETs were purchased from Cayman Chemical Co. (Ann Arbor, Mich.). Methylene chloride, NADPH, EDTA, HPLC-grade acetonitrile, and diethyl ether were purchased from Sigma. HPLC-grade hexane, isopropyl alcohol, and ethanol were obtained from Fisher. ACS-grade ethanol was obtained from Pharmco (Brookfield, Conn.).

The following antibodies are from Cell Signaling Technology, Inc. (Danvers, Mass.): Phospho-mTOR (Ser2448) (D9C2) XP® Rabbit mAb #5536; mTOR Antibody Rabbit polyclonal antibody #2972; Phospho-p70 S6 Kinase (Thr389) Antibody #9205 Rabbit polyclonal antibody; p70 S6 Kinase Antibody #9202 Rabbit polyclonal antibody; Phospho-AMPKα1 (Ser485)/AMPKα2 (Ser491) Antibody #4185 Rabbit polyclonal antibody; AMPKα Antibody #2532 Rabbit polyclonal antibody; Phospho-PKM2 (Tyr105) Antibody #3827 Rabbit polyclonal antibody; PKM2 Antibody #3198 Rabbit polyclonal antibody; Phospho-Stat3 (Tyr705) (D3A7) XP® Rabbit mAb #9145; Stat3 (124H6) Mouse mAb #9139; Phospho-p44/42 MAPK (Erk1/2) (Thr202/Tyr204) (D13.14.4E) XP® Rabbit mAb #4370; p44/42 MAPK (Erk1/2) Antibody #9102 Rabbit polyclonal antibody; and β-Actin (13E5) Rabbit mAb #4970.

The following antibody was from Xenotech, LLC (Kansas City, Kans.) Anti-CYP3A4, rabbit polyclonal antibody, #PWB3A4.

Antibodies used in this study were purchased from Cell Signaling (Danvers, Mass.). Subcutaneous 17-β-estradiol pellets (0.72 mg/60 day) were purchased from Innovative Research of America (Sarasota, Fla.).

Quantitative Immunofluorescence of Breast Cancer TMAs

An annotated tissue microarray (TMA) was obtained from patients enrolled at Yeungnam University College of Medicine (Daegu, Republic of Korea; IRB approved) between 1999 and 2000. The sequentially acquired tissue was de-identified and archival 1 mm tumor cores were arrayed in duplicate (Yeungnam University, Daegu, Korea; IRB approved) (16, 42, 43). Tumors were from 48 consecutive patients who were diagnosed with invasive breast cancer between 1999 and 2000. Quantitative immunofluorescence (AQUA) interrogation of the TMA was performed to evaluate CYP3A4 in cytoplasm and ERα in nuclei. Masking using the CK8 antibody was performed to block out fluorescence signal from the stromal component of tumors. Dual immuno-staining was performed with a FITC-tagged (green) secondary antibody to detect ERα, a Cy5 secondary antibody (red) to detect CYP3A4, and a Cy3 secondary antibody to detect cytokeratin 8 (CK8). DAPI (4′,6-diamidino-2-phenylindole, dihydrochloride) was used to stain nuclei. The original AQUA values for CYP3A4 in cytoplasm, and ERα in nuclei were transformed using the natural logarithm. These values were then averaged across the two AQUA data sets. Pearson's correlation was then calculated between these markers.

LC-ESI/MRM/MS Method for Eicosanoid Quantification.

Samples were submitted to liquid chromatography-electrospray ionization/multiple reaction monitoring/mass spectrometry (LC-ESI/MRM/MS) analysis in a Thermo Electron Quantum Discovery Max triple quadrupole mass spectrometer (Thermo Finnigan, San Jose, Calif.) coupled with an Agilent 1100 HPLC (Santa Clara, Calif.), using argon as the collision gas. Negative ion monitoring was performed at the following diagnostic product ions: 319 m/z→155 m/z for 8,9-EET; 319 m/z→179 m/z for 11,12-EET; 319 m/z→219 m/z for 14,15-EET; 339 m/z→163 m/z for 8,9-[13C20]EET; 339 m/z→233 m/z for 11,12-[13C20]EET; and 339 m/z→259 m/z for 14,15-[¹³C₂₀]EET. Base-line resolution of EET regioisomers was achieved on a Phenomenex Luna C18 (2) reverse phase capillary column (250×0.5 mm, 5-μm particles) using the following mobile phase combinations: isocratic 5% B for 5 min, gradient 5-70% B for 5 min, hold at 70% for 30 min and then 95% for 10 min; A: 0.01% acetic acid in water, B: 0.01% acetic acid in acetonitrile; 10 l/min flow rate. A standard curve was obtained by linear regression of the peak area ratio of authentic EET regioisomers against internal standards. The amount of EETs in samples was calculated according to the standard curve. The (±)-5,6-EET regioisomer was not measured because it undergoes rapid internal degradation.

CYP3A4 Nanodisc-Mediated EET Biosynthesis

In 1 ml of 100 mM HEPES, pH 7.4, 10 mM MgCl2, 0.1 mM dithiothreitol (DTT) buffer, CYP3A4-nanodisc (ND) and cytochrome P450 reductase (CPR) were added to final concentrations 0.27 uM and 1.15 μM, respectively (1:4 molar ratio). The mixture of CYP3A4-ND and CPR was equilibrated for 10 minutes at 37° C. before adding arachidonic acid (55 uM) followed by addition of NADPH (160 μM). The reaction was terminated after 2 minutes by addition of 2 ml of dichloromethane. Samples were centrifuged at 3,000 rpm for 10 min and 0.5 ml of the organic phases were evaporated under a gentle stream of nitrogen. Residues were reconstituted in 20 μl of MeOH containing [¹³C₂₀]-EET internal standards and analyzed by LC-ESI-MS/MS.

EET Extraction from Cells

Cells grown to 50-75% confluence on 150×20-mm plates were washed twice with cold PBS and collected in cold PBS containing 2 μM soluble epoxide hydrolase inhibitor 1471 (a gift from Dr. Bruce Hammock, University of California, Davis) and, after the addition of [¹³C₂₀]EET internal standards, extracted with a 2:1 mixture of chloroform/methanol. After saponification of the organic extracts, extraction of the resulting fatty acids into acidified ethyl ether, and evaporation under N2, the samples were dissolved in MeOH for mass spectrometric analysis as described above.

MCF-7 CYP3A4 shRNA Xenograft Model

The CYP3A4 shRNA cell line 3-18 and scramble control line NT2 were isolated and grown as previously described, derived from MCF-7 cells (parental cells were a gift of Dr. H. Nakshatri, Indiana U.) (Mitra et al., J Biol Chem. 2011; 286:17543-59). The growth of these cell lines in a nude mouse model was performed under IACUC protocol 1302-30326A. Female athymic nude mice (Foxn1^{nu}/Foxn1^{nu}) at 4-6 weeks of age were used. The cell lines were tested for tumor formation in the right mammary fat pad of nude mice, with estradiol supplementation by timed-release pellet. For the MCF-7 xenograft, 3×10⁶ cells in log phase growth were placed in the right 2^{nd} mammary fat pad using a 25-gauge needle on day 0. β-estradiol (E2) was given by subcutaneous 0.72 mg, 60-day release 17β-estradiol pellet implanted the day before tumor implantation. Tumor modeling was performed using Gompertzian curve fitting with a non-zero baseline to allow use of this model. The mammary fat pad was harvested en bloc after animal sacrifice by isoflurane anesthesia followed by cervical dislocation, and the resection specimen was inked and oriented and placed in tissue block holders with sponges. After formalin fixation and paraffin embedment, the blocks were serially sectioned and examined for tumor by H+E staining.

Isolation of MCF7 CYP3A4 Over-Expressing Cell Lines.

CYP3A4 over-expressing lines were isolated by transfecting MCF-7 cells (parental cells were a gift of Dr. H. Nakshatri; Indiana U.) with a pcDNA3.1 vector encoding a myc-His6 tag and the CYP3A4 open reading frame. Transfections were performed using the FuGENE™ transfection reagent according to manufacturer's instruction (Promega, Madison, Wis.) with MCF-7 cells seeded in poly-D-lysine coated 6-well plates. To a 100 mm tissue culture plates, transfection reactions were plated at 500 cells/plate in complete media with G418 at 600 μg/ml for selection. After two weeks, visible and well isolated surviving colonies were picked with the cloning ring/trypsin method and grown in 48 well plate and then 100 mm tissue culture plate when reached 60% confluency. CYP3A4 expression levels of clones were compared by Western blot.

Colocalization of CYP3A4 and Mitochondria in MCF-7 Cells.

MCF-7 cells stably over-expressing CYP3A4 were seeded on fibronectin-coated cover slips and incubated with 100 mM MitoTracker (red, Invitrogen, San Diego, Calif.) in dark for 30 minutes the next day. Cells were fixed with 4% paraformaldehyde in PBS and permeabilized with 0.1% Triton-100. After blocking with donkey serum, fixed cells were probed with polyclonal rabbit anti human CYP3A4 primary antibody (XenoTech LLC, Kansas City, Kans.) followed by wash, blocking and incubation with FITC conjugated anti rabbit IgG secondary antibody (green). Cover slips were then washed, dried and mounted on slides. Slides were observed and fluorescence images were acquired using an Olympus 1X70 microscope fitted with an Olympus DP70 digital camera (Olympus, Tokyo, Japan). Green fluorescent image and red fluorescent image of the same view field were merged with accompanying software DP manager from the manufacturer. Yellow color indicates overlapping of red and green fluorescent light. A pcDNA3 vector control line was analyzed as control. The secondary antibody resulted in no significant background fluorescence in the absence of the primary CYP3A4 antibody.

Measurement of OCR and ECAR

Cells were maintained in growth medium consisting of 10% FBS at 37° C. with 5% CO₂ and seeded at 100,000 cells per well in XF24-well cell culture microplates (Seahorse Bioscience, North Billirica, Mass.). Concentrated stocks of HBB were prepared in DMSO. HBB was diluted to 10× working concentration in XF assay medium (a non-buffered medium including 2 mM L-glutamine but no sodium bicarbonate (buffering agent), glucose, or sodium pyruvate). Assays were performed in the XF Extracellular Flux Analyzer (Seahorse Bioscience that measures uptake and excretion of metabolic end products in real time). Oxygen consumption rate (OCR) and extracellular acidification rate (ECAR) were measured using an XF Assay Kit. OCR is reported in pmoles/minute and ECAR in mpH/minute. Before analysis, the cells were switched from culture medium to XF assay medium. After baseline measurements, 75 μl of HBB prepared in assay medium was injected into each well to reach final working concentrations. After addition of HBB, OCR and ECAR were measured at 18-minute intervals. There were 5 replicates was performed for each data point for MCF-7, MDA-MB-231, T47D and MDA-MB-435 cell lines. There were 6 replicates for the studies of the 3-18 and 4-14 cell lines.

Determination of Cellular ATP by Luminescent Assay

Cells were seeded in 96-well tissue culture plate in 100 μL medium per well. After treatment with either vehicle or testing compound for an appropriate length of time, 50 μL mammalian cell lysis solution was added to each well and the plate was shaken for 5 minutes on an orbital shaker. To each well, 50 μL luminescent substrate and luciferase solution were then added. After incubation in the dark for 10 minutes, the luminescence measured. Cellular ATP levels were then calculated against a standard curve generated with ATP standards of known concentration.

Western Blot Analysis

RIPA extracts of cells were prepared as described previously, in the presence of protease inhibitors and phosphatase inhibitors. Protein concentrations were determined by the micro-bicinchoninic acid (BCA) method, and 30 μg of protein was used for each lane of the SDS-polyacrylamide gel Western blot. Relative protein expression was estimated using GAPDH or β-actin as an internal standard. Quantification was performed by densitometry of x-ray film exposures using an Alpha-Innotec densitometer (Mitra et al., J Biol Chem. 2011; 286:17543-59).

### 3-(4,5-Dimethylthiazol-2-yl)-2,5-diphenyltetrazolium Bromide (MTT) Cell Proliferation Assay

The human breast cancer cell lines MCF-7, T47D, and MDA-MB-231 described above were grown in Minimal Essential Medium (MCF-7), RPMI 1640 with insulin (0.2 U/mL) and 1 mM Na pyruvate (T47D) or DMEM containing 10% fetal bovine serum (FBS) (MDA-MB-231), which was defined as complete medium (CM). Cells were grown at 37° C. in a 5% CO₂ incubator. The cells were incubated with vehicle or biguanide for 48, 72 or 96 h. MTT was then added (5 mg/ml), and after 2 h of incubation, the plates were centrifuged. The supernatant was removed; DMSO was added, and the absorbance was read at 540 nm with a BioTek 96-well plate reader.

Binding of Biguanides to CYP3A4 Nanodiscs

Substrate titration experiments were performed at 1 μM concentration of CYP3A4 in Nanodiscs using a Cary 300 spectrophotometer (Varian, Lake Forest, Calif.) at 21° C. Incorporation of CYP3A4 in POPC nanodiscs was done following standard protocols described earlier (Luthra et al., Methods Mol Biol. 2013; 987:115-27).

CYP3A4 Co-Crystal Formation and X-Ray Crystallography

Δ(3-22)CYP3A4 protein was co-crystallized with metformin at room temperature by a microbatch method under oil. CYP3A4 (115 mg/ml) in 50 mM phosphate, pH 7.4, 20% glycerol, and 100 mM NaCl was incubated for 20 min with a 40-fold excess of metformin. Prior to mixing with CYP3A4, pH of the aqueous metformin solution was adjusted to 7.0 with concentrated HCl. After removal of the precipitate by centrifugation, 0.4 microliters of the protein solution was mixed with 0.4 microliters of 12% PEG 3350 and 0.1 M sodium acetate pH 7.0, and the drop was covered with paraffin oil. Crystals were harvested 3 days later and cryoprotected with Paratone-N before freezing in liquid nitrogen. X-ray diffraction data were collected at −170C at the Stanford Synchrotron Radiation Lightsource (SSRL) beamline 7-1. The atomic coordinates were deposited in the Protein Data Bank with the ID code 5G5J.

Recombinant Microsomal CYP-Mediated EET Biosynthesis

CYP3A4 or 2C8 Supersomes™ (BD Biosciences) were incubated at 37° C. for 30 min in the presence of AA (5 μM) in 0.05 mm Tris-HCl, pH 7.4, containing 1 mm EDTA and 1 mm NADPH. Reactions were terminated by adding methylene chloride (0.5 ml). Samples were centrifuged at 3,000 rpm for 10 min and 150 μl of the organic phases were evaporated under nitrogen. Samples were reconstituted in 20 μl of MeOH containing [¹³C₂₀]EET internal standards and submitted to a Thermo Electron Quantum Discovery Max triple quadrupole mass spectrometer (Thermo Finnigan, San Jose, Calif.) coupled with an Agilent 1100 HPLC (Santa Clara, Calif.) for analysis. Reaction extracts were chromatographed under the conditions described above, and ions of 319 m/z, corresponding to AA monooxygenation products, were selectively monitored in negative mode. Concentrations of EETs generated by CYP Supersomes™ were calculated by linear regression against standard curve generated with EET standards.

Docking of HBB and Other Candidate Neo-Biguanides

All molecules were initially constructed using SYBYL-X 2.0 (Tripos, Inc.). Energy minimization of these compounds was performed using the Tripos forcefield with Gasteiger-Hückel charges for a maximum of 10000 iterations subject to a termination gradient of 0.001 kcal/(mol·Å).

Predicted bound configurations for these structures were obtained using Surflex-Dock (SYBYL-X 2.0, Tripos, Inc.), with our CYP3A4/metformin cocrystallized complex structure. The co-crystallized ligand metformin was used to guide the protomol generation process. Docked poses were ranked by total Surflex-Dock score expressed as −log(K_{d}). Threshold and bloat parameters were set to 0.5 and 0, respectively. The maximum number of conformations per compound fragment and the maximum number of poses per molecule were both set to twenty, and the maximum allowable number of rotatable bonds per structure was limited to 100. Post-dock minimizations were carried out on each ligand to optimize predicted configurations in the receptor site.

All calculations were carried out within the SYBYL-X 2.0 (Tripos, Inc.) environment on Minnesota Supercomputing Institute (MSI) Dell Precision T7400 workstations running under the CentOS 6.2 operating system. Visualizations were obtained using PyMOL, Version 1.5.0.4 (Schrödinger, LLC) in Mac OS X version 10.6.8.

Synthesis of Neo-Biguanides

N1-Hexyl-N5-benzyl biguanide mesylate (HBB mesylate) synthesis (7). n-Hexylamine (27 ml, 204 mmol) was mixed with 150 ml of n-butanol before adding sodium dicyanamide (20 g, 225 mmol) and 19 ml of concentrated HCl. The solution was refluxed for 24 h followed by evaporation of butanol to yield a sticky white residue, which was taken up in dichloromethane, washed with water, and extracted with 3×CH₂Cl₂. The combined organics were dried over Na₂SO₄, filtered, and concentrated under reduced pressure to a white solid. Additional white solid was filtered off of the aqueous layer, filtered, and dried. The combined white solids were intermediate 1-hexyl-3-cyanoguanidine (54% yield), which was used in the next step without further purification. NMR data for structure verification of 1-Hexyl-3-cyanoguanidine: ¹H NMR (400 MHz, DMSO-d₆) δ 7.4-6.15 (br m, 3H), 3.01 (q, J=6.59 Hz, 2H), 1.39 (m, 2H), 1.23 (m, 6H), 0.86 (t, J=6.82 Hz, 3H); ¹³C NMR (400 MHz, DMSO-d₆) δ 161.1, 118.4, 40.6, 30.8, 28.8, 25.8, 22.0, 13.8. Next, 2.56 ml of benzylamine (23.47 mmol) was dissolved in 30 ml of n-butanol with 1.78 ml of concentrated hydrochloric acid. After stirring for 30 minutes at ambient temperature, 3.59 g (21.34 mmol) of the product from the previous step was mixed with 15 ml of n-BuOH, and the solution was added to the benzylamine-HCl mixture. The reaction mixture was refluxed for 24 h before distilling off the butanol and concentrating the remaining residue to a solid under reduced pressure. The biguanide was purified by flash column chromatography with silica gel to yield 2.1 g of N1-hexyl-N5-benzyl biguanide (36% from cyanoguanidine). N1-Hexyl-N5-benzyl biguanide ¹H NMR (400 MHz, DMSO-d₆) δ 7.92-7.38 (br m, 2H), 7.38-7.20 (m, 5H), 7.20-6.35 (br m, 3H), 4.33 (d, J=5.92 Hz, 2H), 3.03 (q, J=6.5 Hz, 2H), 1.40 (br s, 2H), 1.22 (br s, 6H), 0.84 (t, J=6.5 Hz, 3H). HRMS calculated [M+H]⁺ 276.2181, found 276.2183. Next 1.23 g (4.46 mmol) of the resultant biguanide in 30 ml of dichloromethane was subjected to 5 ml of a 0.9M methanesulfonic acid solution in dichloromethane, providing 1.66 g (100% yield from HBB) of the desired HBB-mesylate salt as a fluffy white solid after concentration. NMR data for structure verification of N1-Hexyl-N5-benzyl biguanide mesylate: ¹H NMR (400 MHz, DMSO-d₆) δ 10.0-7.34 (br m, 4H), 7.34-7.28 (m, 5H), 6.93 (br s, 1H), 5.80 (br s, 1H), 4.40 (br s, 2H), 3.12 (br s, 2H), 2.36 (s, 3H), 1.47 (br s, 2H), 1.25 (br s, 6H), 0.85 (m, 3H). The calculated HRMS [M+H]⁺ 276.2181, and the found HMRS was 276.2183.

(N1-Hexyl-N5-(1H-imidazol-2-yl)methyl)biguanidine) trihydrochloride (HIB) synthesis. (1H-Imidazol-2-yl)methanamine dihydrochloride (250 mg, 1.47 mmol) was dissolved in 40 ml of n-butanol. After adding 1-hexyl-3-cyanoguanidine (296 mg, 1.75 mmol), the reaction mixture was refluxed for 24 h. The n-butanol was removed completely under reduced pressure, and the resulting residue was dissolved in dry methanol and cooled to 0° C. An HCl solution in 1,4-dioxane was added, and the reaction was stirred for 20 min at 0° C. The solvent was then removed under reduced pressure. To the residue, dry CH₂Cl₂ and a few drops of dry methanol were added and cooled overnight in a refrigerator. The resulting salt was filtered and washed with cool dry CH₂Cl₂ to afford 143 mg (26%) of (N1-hexyl-N5-(1H-imidazol-2-yl)methyl) biguanidine) trihydrochloride. NMR data for structure verification of HIB were ¹H NMR (400 MHz, DMSO-d₆) δ 14.45 (br s, 1H), 7.81 (br s, 2H), 7.56 (s, 2H), 7.17 (br s, 2H), 5.75 (s, 1H), 4.66 (br s, 2H), 3.10-2.89 (m, 2H), 1.56-142 (m, 2H), 1.38-1.12 (m, 6H), 0.87-0.84 (m, 3H).

N1-hexyl-N5-(pyridin-4-ylmethyl) biguanide (HPB) synthesis. To a vial was added 424 mg (3.92 mmol) of 4-(aminomethyl)pyridine and 5 ml of n-butanol before adding 0.65 ml of concentrated hydrochloric acid, and the mixture was stirred for 30 minutes at ambient temperature. The previously prepared 1-hexyl-3-cyanoguanidine (600 mg, 3.566 mmol) was added as a solution in 2.5 ml of n-butanol before refluxing for 24 hours. The solvent was evaporated at 60° C. and purified by flash column chromatography with silica gel (dichloromethane:methanol) to yield 130 mg (13%) of final product (N1-hexyl-N5-(pyridin-4-ylmethyl) biguanidine) as a dark yellow oil. ¹H NMR (400 MHz, DMSO-d₆) δ 10.22 (br s, 1H), 8.90 (br s, 1H), 8.52-8.47 (m, 2H), 8.46 (br s, 2H), 8.23 (br s, 1H), 7.28 (d, 2H, J=5.64 Hz), 4.33 (m, 2H), 3.21 (m, 2H), 1.51-1.46 (m, 2H), 1.26 (m, 6H), 0.87 (m, 3H). ¹³C NMR (400 MHz, DMSO-d₆) δ 159.9, 154.9, 154.2, 141.3, 124.8, 42.4, 40.9, 30.7, 27.8, 25.5, 21.9, 13.8. LRMS calculated was [M+MeCN+H]⁺ 318.3, LMRS found was 318.4 and 321.2 ([M+2Na+H]⁺). The biguanidine product 50 mg (0.18 mmol) was subjected to 0.1 M methanesulfonic acid in water (1.81 ml) to provide the 30 mg (45%) of desired HPB dimesylate as a sticky yellow solid after concentration and purification by silica gel flash column chromatography (dichloromethane:methanol). NMR structure verification for N1-hexyl-N5-(pyridin-4-ylmethyl) biguanidine dimesylate: ¹H NMR (40 MHz, DMSO-d₆) δ 10.32 (br s, 1H), 8.85 (br s, 1H), 8.81 (br s, 2H), 8.46 (br s, 2H), 8.23 (br s, 1H), 7.83 (br s, 2H), 4.56 (br s, 2H), 3.23 (br m, 2H), 2.33 (s, 6H), 1.48 (m, 2H), 1.26 (m, 6H), 0.85 (m, 3H).

JC-1 Mitochondrial Membrane Potential Dye Staining for Fluorescence Microscopy:

Cells were grown in chamber slides at 37° C. with 5% CO₂ and treated with DMSO or HBB for 4 hours before JC-1 dye was added for the MCF-7 or MDA-MB-231 cell line. After 15 minutes incubation, slides were observed with a fluorescence microscope. Images of red fluorescence (590 nm) and green fluorescence (530 nm) were acquired using an Olympus 1X70 microscope fitted with an Olympus DP70 digital camera (Olympus, Tokyo, Japan).

MCF-7 and MDA-MB-231 Xenograft Models

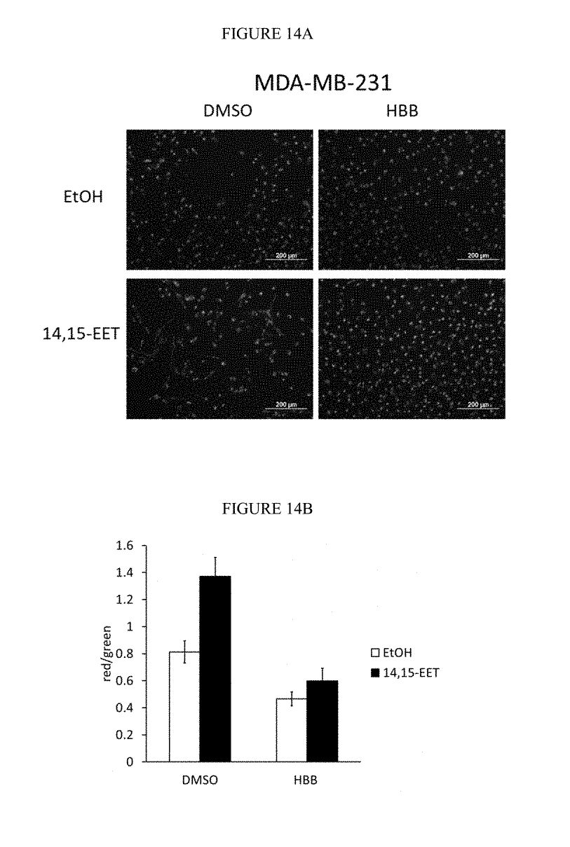

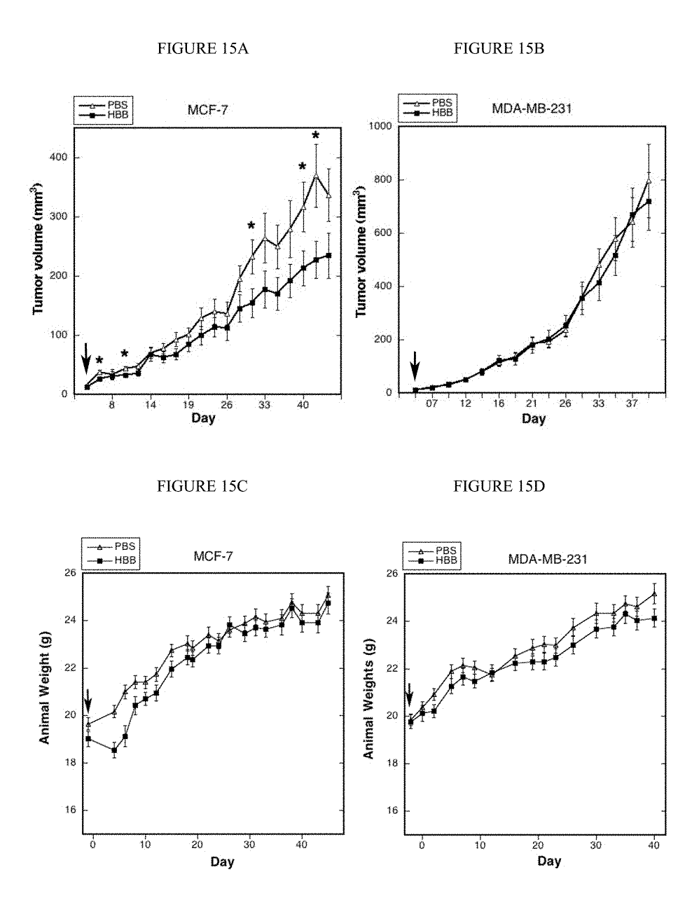

The animal studies were performed according to an IACUC-approved protocol 1505-32594A and monitoring by veterinary staff. For the xenograft studies, female athymic nude mice (Foxn1^{nu}/Foxn1^{nu}) at 4-6 weeks of age were used. The mice were weighed twice weekly from receipt to the end of the study and nutrition and behavior were monitored by veterinary staff and technicians. Tumorigenic MCF-7 (gift of Dr. Deepali Saachdev; U. Minnesota) and MDA-MB-231 (gift of Dr. Harikrishna Nakshatri; Indiana U.) cell lines responsive to EETs were previously published (Mitra et al., J Biol Chem. 2011; 286:17543-59). The MCF-7 cells were tested and confirmed to be estradiol responsive in vitro (data not shown). For the MCF-7 xenograft dosed at 6 mg/kg (FIG. 7), 5.1×10⁶ cells in log phase growth were placed in the right 2^{nd} mammary fat pad using a 25-gauge needle on day 0. For the MCF-7 xenograft dosed at 4 mg/kg (FIG. 14) 3.8×10⁶ cells in log phase were injected in the right 2^{nd} mammary fat pad. 17-β-estradiol (E2) was given in drinking water at 1 μM beginning the day of tumor implantation. Randomization was performed prior to treatment using the web program www.randomizer.org. Tumor measurements were taken with a digital caliper twice weekly and thereafter at the same time that weights were recorded. Tumor volumes were determined by the formula: volume=length×[width]²/2. Tumors were collected from each mouse after sacrifice and endpoint tumor weights were measured. For the MDA-MB-231 xenograft model, the procedures were similar, but mice were injected with 1×10⁶ cells in the right 2^{nd} mammary fat pad and no estradiol was used in the drinking water. In both experiments, the mice were randomized the day after tumor cell injection. Euthanasia was performed by isoflurane anesthesia followed by cervical dislocation for all mice at the study endpoint unless an individual mouse met euthanasia criteria prior to this time. Tumors were harvested after sacrifice and bisected, with half placed in formalin for histology and half in (Optimal Cutting Temperature) OCT compound for reverse phase array analysis.

The MTD for daily dosing was found by two-fold dose escalation and the finding that 8 mg/kg/day ip in cohorts of 5 tumor-bearing mice resulted in animal deaths within 3 days and was 4 mg/kg/day ip. The initial studies were performed with ip treatment with HBB at the MTD or with PBS beginning on day 1, indicated by the arrow (FIG. 14a). N=20 mice per arm for each study; error bars represent SEM. Significant weight loss was observed for the MCF-7 and MDA-MB-231 models after initiation of daily HBB dosing at 4 mg/kg/day ip, but weight loss was no more than 8% for the MCF-7 model noted on day 4 (P=0.00057) and recovered by the end of the study (P=0.59) (FIG. 14c,d). For the MDA-MB-231 model end point weight loss was 2.5% (P=0.31). The plasma concentration of HBB was measured 1 hour after ip dosing was 0.53 μM 1 hour after the daily dose, (4 mg/kg/day ip). Detailed pharmacokinetics will be performed in a follow-up study, but may require tissue measurements because tissue levels of metformin are much higher than plasma levels (Choi Y H, Lee M G. Xenobiotica. 2012; 42:483-95; Chandel et al., Cell Metab. 2016; 23:569-70).

Dose and Schedule Optimization of HBB in the MCF-7 Xenograft Model

In the HBB dose optimization model, mammary fat pad injection was performed with the MCF-7 cell line as described above, and tumors were allowed to grow until an average tumor volume of 50 mm³. Mice were randomized, and tumor growth was monitored. Estradiol (1 μM) was present in the drinking water as above. Cremophor was administered weekly in the vehicle and HBB groups so that paclitaxel could be used in subsequent chemotherapy synergy studies (unpublished data). Mice were dosed at 6 mg/kg/day ip 4 days of 7 with no more than 2 consecutive days dosing and weighed daily. The weights declined by >5% vs. control after three consecutive days of dosing and the dosing schedule was changed to 4 times weekly, with no more than two consecutive days dosing. Weight loss occurred most significantly after 3 days of consecutive dosing, with a maximum weight loss of 11% (P<0.0001) in the HBB treatment group, but then recovered rapidly and weight gain continued such that at the end of the experiment (day 38) animal weights were 6% lower than control in the HBB treatment group (P=0.0042). Tumors were harvested at the endpoint of the study following sacrifice by isoflurane anesthesia followed by cervical dislocation. Organs were harvested for histopathological assay of end organ toxicity. There was no hepatic or cardiac toxicity found by histopathological examination of post-mortem liver and cardiac tissue of HBB-treated mice as compared with vehicle control mice (n=5 for each condition; data not shown)

Reverse Phase Protein Array (RPPA) Analysis of Xenograft Samples: Xenograft Tissue Processing

Frozen xenograft tissue samples were embedded in OCT, and 8 μm sections were obtained using a cryostat. Ten sections per sample were briefly fixed in 70% ethanol containing protease inhibitors (Complete Mini EDTA-free, Roche), dehydrated in 95% ethanol, followed by 100% ethanol and finally xylene and then lysed directly from slides in an appropriate volume extraction buffer containing 50% Tissue Protein Extraction Reagent (T-PER, Thermo Fisher Scientific), 47.5% 2× Tris-Glycine SDS sample buffer (Invitrogen), and 2.5% β-mercaptoethanol (Thermo Fisher Scientific). The resulting whole tissue lysates were boiled for 8 min at 100° C. and then printed onto a nitrocellulose slide (Avid, Grace Biolabs) along with a BSA protein concentration curve to estimate total protein concentration in each lysate. Total protein levels were assessed in each sample by staining with Sypro Ruby Protein Blot Stain (Invitrogen) according to manufacturer's instructions.

RPPA Printing and Analysis

The total protein concentration in each sample was estimated by printing onto a nitrocellulose slide along with a BSA standard concentration curve, and total protein levels were assessed by staining with Sypro Ruby Protein Blot Stain (Invitrogen) according to manufacturer's instructions. Tissue lysates were diluted to 250 μg/ml in extraction buffer and stored at −80° C. prior to printing of arrays. RPPA printing and analysis of xenograft samples was conducted as previously described (Wulfkuhle et al., Clin Cancer Res. 2012; 18:6426-35). Antibody staining intensities were quantified using the MicroVigene v5 Software Package (Vigenetech). Signaling pathway activation was evaluated by staining the arrays with 156 antibodies against signaling endpoints, mainly phosphorylated and cleaved protein products. Before use for RPPA analysis, antibody specificity was confirmed by Western blot and analysis as previously described (Wulfkuhle et al., Clin Cancer Res. 2012; 18:6426-35).

Determination of Oxygen Substrate Kₘ for CYP3A4

Human recombinant CYP3A4 Supersomes™ (BD Bioscience)-catalyzed epoxygenation of arachidonic acid (AA, 100 μM) reaction was performed at 37° C. in the presence of a NADPH regenerating system. Oxygen concentration of the reaction solution was varied and rates of oxygen consumption were recorded with a YSI 5300 biological oxygen monitor (Yellow Springs Instruments, Yellow Springs, Ohio). Lineweaver Burke analysis was performed to calculate the oxygen Michaelis-Menten constant (Kₘ).

Determination of Rate of Cytochrome P450 Reductase (CPR)-Mediated Reduction of Cytochrome c

In a quartz cuvette, reduction of cytochrome c reaction was initiated by addition of CPR (final concentration 0.2 mg/mL) to 1 mL potassium phosphate buffer (0.3 M, pH=8.3) containing cytochrome c (62 mM) and NADPH 50 (mM). Absorbance at 550 nM was monitored real time by a Cary 50 UV-Vis Spectrophotometer (Agilent, Santa Clara, Calif.).

TABLE 1
Data collection and refinement statistics
for the CYP3A4-metformin structure

| Space group | I222 | |
|---|---|---|
| Unit cell parameters | a = 77 Å, b = 101 Å, c = 128 Å, α = β = γ = 90° | |
| Resolution range | 77.4-2.6 | (2.72-2.60)^{a} |
| Total reflections | 68,206 | |
| Unique reflections | 15,443 | |
| Redundancy | 4.4 | (4.5) |
| Completeness | 98.2 | (99.6) |
| Average I/σI | 10.5 | (2.0) |
| R_{merge} | 0.068 | (0.559) |

| | |
|---|---|
| R/R_{free}^{b} | 21.2/28.1 |
| r.m.s. deviations | |
| Bond lengths, Å | 0.010 |
| Bond angles, ° | 1.9 |

^{a}Values in brackets are for the highest resolution shell.
^{b}R_{free} was calculated from a subset of 5% of the data that were excluded during refinement.

TABLE 2
Structures and Docking Scores of 19 Biguanide Compounds

| | | | Total Score |
|---|---|---|---|
| | Comp | | [CYP3A4 |
| Structures | ID | Name/ID | Active Site] |
| 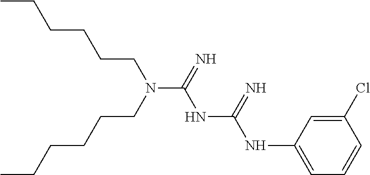 [SMILES: CCCCCCN(CCCCCC)C(=N)NC(=N)Nc1cccc(Cl)c1] | 1 | Ex_34 | 8.84 |
| 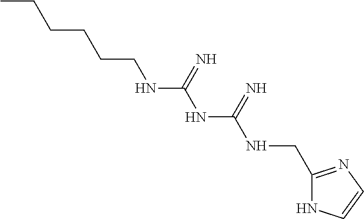 [SMILES: CCCCCCNC(=N)NC(=N)NCc1ncc[nH]1] | 2 | HIB | 7.88 |
| 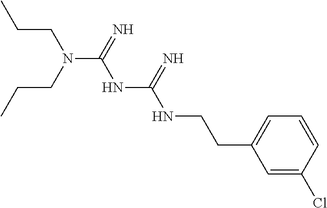 [SMILES: CCCN(CCC)C(=N)NC(=N)NCCc1cccc(Cl)c1] | 3 | Ex_38 | 7.12 |
| 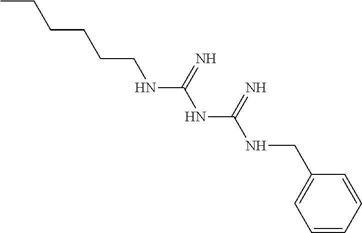 [SMILES: CCCCCCNC(=N)NC(=N)NCc1ccccc1] | 4 | HBB | 7.11 |
| 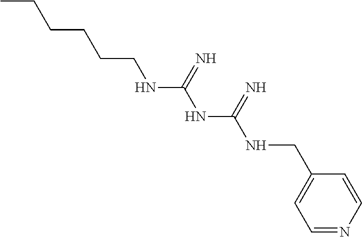 [SMILES: CCCCCCNC(=N)NC(=N)NCc1ccncc1] | 5 | HPB | 6.90 |
| 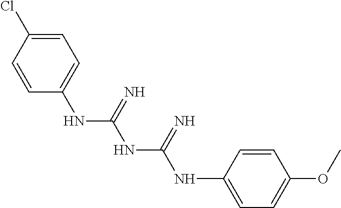 [SMILES: COc1ccc(NC(=N)NC(=N)Nc2ccc(Cl)cc2)cc1] | 6 | Ex_7 | 6.70 |
| 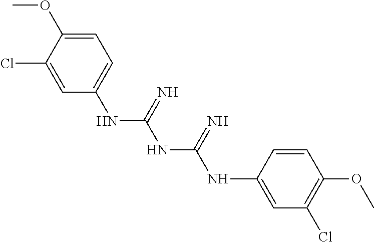 | 7 | Ex_8 | 6.61 |
| 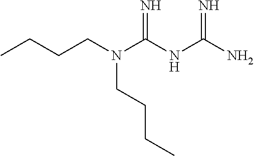 | 8 | N,N-dibutyl- biguanide | 6.53 |
| 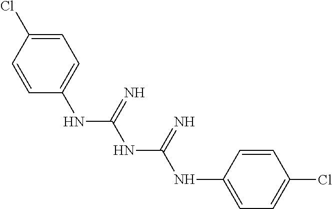 [SMILES: N=C(NC(=N)Nc1ccc(Cl)cc1)Nc1ccc(Cl)cc1] | 9 | Ex_5 | 5.48 |
| 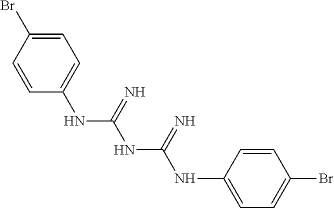 [SMILES: N=C(NC(=N)Nc1ccc(Br)cc1)Nc1ccc(Br)cc1] | 10 | Ex_11 | 5.15 |
| 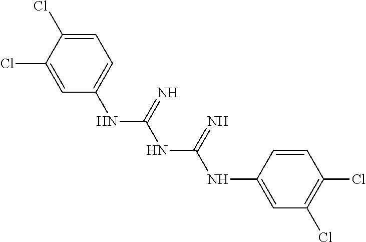 [SMILES: N=C(NC(=N)Nc1ccc(Cl)c(Cl)c1)Nc1ccc(Cl)c(Cl)c1] | 11 | Ex _9 | 5.02 |
| 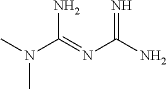 | 12 | metformin | 4.75 |
| 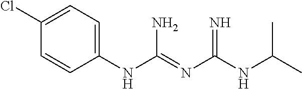 [SMILES: CC(C)NC(=N)/N=C(\N)Nc1ccc(Cl)cc1] | 13 | Proguanil | 4.73 |
| 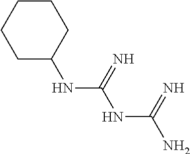 [SMILES: N=C(N)NC(=N)NC1CCCCC1] | 14 | 1- cyclohexyl- biguanide | 4.60 |
| 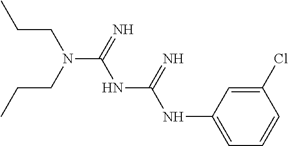 [SMILES: CCCN(CCC)C(=N)NC(=N)Nc1cccc(Cl)c1] | 15 | Ex_31 | 4.44 |
| 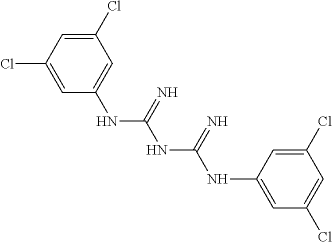 [SMILES: N=C(NC(=N)Nc1cc(Cl)cc(Cl)c1)Nc1cc(Cl)cc(Cl)c1] | 16 | Ex_10 | 4.37 |
| 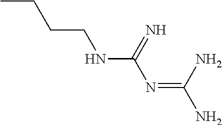 | 17 | buformin | 4.37 |
| 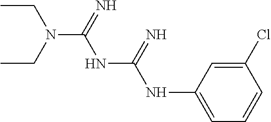 [SMILES: CCN(CC)C(=N)NC(=N)Nc1cccc(Cl)c1] | 18 | Ex_30 | 4.36 |
| 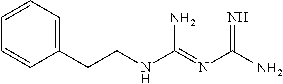 [SMILES: N=C(N)/N=C(\N)NCCc1ccccc1] | 19 | phenformin | 3.80 |

TABLE 3
Inhibition of the MCF-7 Cell Line By Biguanides

| | IC₅₀ | |
|---|---|---|
| Biguanide | MCF-7 (mM) | Dock Score |
| buformin | >10 | 4.37 |
| phenformin | 7 | 3.80 |
| metformin | 5 | 4.75 |
| HIB | 0.500 | 7.88 |
| HPB | 0.040 | 6.90 |
| HBB | 0.020 | 7.11 |

TABLE 4
HBB IC₅₀ for Breast Cancer Cell Lines
and a Non-transformed Breast Cell Line

| Cell Line | IC₅₀ (μM) | Description |
|---|---|---|
| MCF-7 | 20.1 | ER+[PR+]HER2− |
| T47D | 25.1 | ER+[PR+] |
| MDA-MB-231 | 20.5 | Triple negative |
| MDA-MB- | 12.5 | Triple negative |
| 435/LCC6 | | |
| MCF-10A | 20.5 | ER−[PR−] |

TABLE 5A
Reverse Phase Protein Microarray (RPPA) Data of
Protein Expression in MCF-7 Xenograft Endpoint
Analysis, treated with HBB 4 mg/kg/day vs. PBS

| Antibody | fold change | p-value |
|---|---|---|
| mTOR | 0.497 | 4.06E−06 |
| PKC zeta/lambda T410/403 | 0.546 | 0.00012 |
| Stat 5 Y694 | 0.558 | 0.01166 |
| Src Y527 | 0.675 | 0.01476 |
| CREB S133 | 0.626 | 0.02485 |
| AXL | 0.566 | 0.03666 |
| ATF2 T69/71 | 0.713 | 0.03727 |
| B-Raf S445 | 0.764 | 0.04438 |
| PKM2 Y105* | 2.13 | 0.04943 |
| PDK1 S241 | 0.712 | 0.05099 |
| ERK 1-2 T202/Y204 | 0.423 | 0.05208 |
| Src family Y416 | 0.471 | 0.05235 |
| AMPKbeta S108 | 0.665 | 0.05372 |
| EGFR Y992 | 0.803 | 0.05551 |
| RAS TOTAL | 1.543 | 0.05605 |
| 4EBP1 T70 | 0.595 | 0.06172 |
| PTEN | 0.726 | 0.06264 |
| FKHR T24/FKHRL1 T32 | 0.698 | 0.06369 |
| c-Abl T735 | 0.776 | 0.06543 |
| ERK TOTAL | 0.791 | 0.07624 |
| HIF-1 alpha | 2.406 | 0.08765 |
| Bax | 0.807 | 0.08937 |
| MEK 1/2 S217/221 | 0.770 | 0.08947 |
| LIMK1 T508/LIMK2 T505 | 0.795 | 0.09883 |
| Rb S780 | 0.674 | 0.09943 |
| EGFR | 0.857 | 0.10532 |
| p27 T187 | 0.685 | 0.11640 |
| Chk1 S345 | 1.290 | 0.11702 |
| XIAP | 0.835 | 0.11781 |
| ATR S428 | 0.810 | 0.12101 |
| PTEN S380 | 0.783 | 0.12454 |
| Aurora A T288/B T232/C T198 | 0.646 | 0.13373 |
| NF-kappaB p65 S536 | 0.817 | 0.13708 |
| Beclin 1 | 0.881 | 0.13771 |
| Shc Y317 | 0.792 | 0.14424 |
| PDL1 | 34.857 | 0.15527 |
| Heregulin | 20.774 | 0.15553 |
| SMAD2 S245/250/255 | 0.760 | 0.15979 |
| FADD S194 | 0.672 | 0.16000 |
| ErbB3/HER3 Y1289 | 1.268 | 0.16515 |
| SGK1 S78 | 1.318 | 0.16638 |
| PKC delta/theta S643/S676 | 0.671 | 0.16963 |
| Cl Caspase 9 D330 | 1.244 | 0.17809 |
| GRB2 | 0.830 | 0.17845 |
| Cyclin A | 1.779 | 0.18436 |
| c-ErbB2/HER2 | 1.938 | 0.18787 |
| HSP70 | 1.453 | 0.18871 |
| c-Kit Y703 | 4.550 | 0.19773 |
| E-Cadherin | 0.669 | 0.19821 |
| ER TOTAL | 1.687 | 0.19867 |
| GSK-3alpha/beta S21/9 | 0.791 | 0.20402 |
| Cyclin D1 (G124-326) | 1.277 | 0.20492 |
| AKT T308 | 0.793 | 0.20650 |
| ErbB3/HER3 Y1197 | 1.223 | 0.20996 |
| c-Met TOTAL | 0.877 | 0.21840 |
| AMPK T172* | 0.598 | 0.22491 |
| IL6 | 1.260 | 0.22733 |
| BAD | 2.424 | 0.22767 |
| Elk S383 | 1.402 | 0.23565 |
| p70 S6 Kinase S371 | 0.762 | 0.23820 |
| Cofilin S3 | 0.443 | 0.26158 |
| p70 S6 Kinase T389 | 0.773 | 0.26563 |
| BIM | 0.727 | 0.28080 |
| BAD S112 | 0.833 | 0.28336 |
| c-Abl Y245 | 0.846 | 0.29500 |
| MCSFR Y723 | 1.228 | 0.29557 |
| p70S6 Kinase | 0.674 | 0.29717 |
| Cyclin D1 T286 | 0.611 | 0.29743 |
| ErbB2/HER2 Y1248 | 1.239 | 0.30139 |
| Ret Y905 | 0.810 | 0.30393 |
| Tuberin/TSC2 Y1571 | 1.208 | 0.30664 |
| PDGFR alpha Y754 | 1.144 | 0.32948 |
| PDGFR beta | 0.705 | 0.34288 |
| Cl Caspase 9 D315 | 1.151 | 0.35224 |
| Cl Caspase 3 D175 | 1.109 | 0.35609 |
| Jak2 Y1007 | 0.881 | 0.35774 |
| EGFR Y1148 | 0.891 | 0.36017 |
| Insulin Receptor Beta | 1.261 | 0.36049 |
| 4EBP1 T37/46 | 1.261 | 0.36180 |
| Cyclin B1 V152 | 1.197 | 0.36334 |
| p90RSK T359/S363 | 0.884 | 0.36398 |
| VEGFR2 Y996 | 0.828 | 0.37812 |
| FOXO1 S256 | 0.854 | 0.37996 |
| PAK1 S199/204-PAK2 S192/197 | 1.221 | 0.38467 |
| Ephrin A3 Y799/A4 Y799/A5 Y833 | 1.149 | 0.38634 |
| AMPK total* | 1.69 | 0.38955 |
| TNF alpha | 1.186 | 0.39198 |
| Smad1 Ser463/465-Smad5 Ser463/ | 0.900 | 0.39548 |
| 465-Smad9 Ser465/467 | | |
| Ros Y2274 | 1.201 | 0.41105 |
| eIF4G S1108 | 0.791 | 0.41576 |
| BCL-2 | 0.825 | 0.42387 |
| MDM2 S166 | 0.868 | 0.43319 |
| KI67 | 0.731 | 0.43498 |
| Bak | 0.789 | 0.43906 |
| AMPKalpha S485 | 1.126 | 0.44452 |
| B-RAF | 1.130 | 0.48249 |
| Bcl_xL | 0.909 | 0.51244 |
| Histone H3 S28 | 0.898 | 0.52065 |
| Vimentin | 0.896 | 0.52069 |
| LKB1 S428 | 1.269 | 0.54363 |
| p27 Kip1 | 0.925 | 0.55258 |
| LC3B | 1.097 | 0.55276 |
| PDGFR beta Y716 | 0.887 | 0.55836 |
| eNOS/NOS III S116 | 0.902 | 0.56296 |
| IkappaB-alpha S32/36 | 1.130 | 0.56299 |
| A-Raf S299 | 0.862 | 0.60198 |
| ALK | 1.079 | 0.60202 |
| PUMA | 0.919 | 0.60825 |
| ErbB3/HER3 | 0.851 | 0.61578 |
| IGF1R Y1135/36-IR Y1150/51 | 1.101 | 0.63505 |
| pYAP S127 | 0.931 | 0.63860 |
| TGF-beta | 0.952 | 0.64319 |
| EGFR Y1068 | 1.089 | 0.66316 |
| Musashi | 0.898 | 0.66464 |
| HSP27 S82 | 0.877 | 0.68069 |
| MARCKS S152/156 | 1.071 | 0.68194 |
| c-Myc | 0.879 | 0.69037 |
| 4EBP1 S65 | 1.051 | 0.71401 |
| S6 Ribosomal Protein S240/244 | 0.817 | 0.72146 |
| Cleaved PARP D214 | 1.277 | 0.72360 |
| ErbB2/HER2 Y877 | 0.951 | 0.72915 |
| Stat 1 Y701 | 0.937 | 0.73301 |
| HSP90a T5/7 | 0.945 | 0.74763 |
| ALK Y1604 | 0.954 | 0.75487 |
| Stat3 Y705 | 0.936 | 0.75525 |
| EGFR Y1173 | 0.954 | 0.76609 |
| ER alpha S118 | 0.964 | 0.77470 |
| Acetyl-CoA Carboxylase S79 | 0.927 | 0.78476 |
| mTOR S2448 | 1.034 | 0.78872 |
| PKA C T197 | 0.907 | 0.79020 |
| CrkL Y207 | 0.962 | 0.79282 |
| BAD S136 | 0.958 | 0.80382 |
| FKHRL1 S253 | 0.960 | 0.82671 |
| Met Y1234/1235 | 1.033 | 0.83038 |
| PAK1 T423/PAK2 T402 | 1.028 | 0.85193 |
| Ras_GRF1 | 1.021 | 0.87655 |
| Actin beta | 1.000 | 0.87780 |
| SAPK/JNK T183/Y185 | 1.033 | 0.88041 |
| AKT S473 | 1.042 | 0.88276 |
| ATM S1981 | 1.034 | 0.89431 |
| STAT3 S727 | 1.040 | 0.90290 |
| PLK1 T210 | 1.018 | 0.90356 |
| FAK Y576/577 | 1.021 | 0.90772 |
| c-Raf S338 | 1.016 | 0.91193 |
| IRS-1 S612 | 0.952 | 0.91407 |
| IRS1 | 1.014 | 0.91710 |
| IGF-1R Y1131/IR Y1146 | 1.019 | 0.92565 |
| IGFBP7 | 0.992 | 0.93514 |
| p53 S15 | 0.990 | 0.94255 |
| p90RSK S380 | 1.016 | 0.95057 |
| PKM2 total* | 1.01 | 0.97680 |
| p38 MAP Kinase T180/Y182 | 1.011 | 0.97691 |
| p53 | 0.997 | 0.98379 |
| p70 S6 Kinase T412 | 0.998 | 0.99286 |
| ATP CItrate Lyase S454 | 1.000 | 0.99848 |
| Histone H3 S10 | No Data | No Data |

Results were normalized using a β-actin probe antibody.
*Indicates tested subsequently in a separate hypothesis driven RPPA analysis of the same tumor specimens and normalized to a separate β actin probe.

TABLE 5B
Reverse Phase Protein Microarray (RPPA) Data of Protein
Expression in MDA-MB-231 Xenograft Endpoint Analysis,
treated with HBB 4 mg/kg/day vs. PBS

| Antibody | fold change | p-value |
|---|---|---|
| Cl Caspase 9 D315 | 1.437 | 0.00008 |
| ALK | 1.368 | 0.00712 |
| Ephrin A3 Y799/A4 Y799/A5 Y833 | 1.553 | 0.00793 |
| Cl Caspase 9 D330 | 1.492 | 0.01260 |
| AKT T308 | 1.987 | 0.01460 |
| ERK TOTAL | 1.242 | 0.01783 |
| AMPK T172* | 3.561 | 0.01889 |
| EGFR Y1173 | 1.388 | 0.01964 |
| EGFR Y1068 | 1.557 | 0.02112 |
| NF-kappaB p65 S536 | 1.655 | 0.02204 |
| ErbB2/HER2 Y1248 | 1.717 | 0.02215 |
| FKHRL1 S253 | 1.671 | 0.02342 |
| ErbB3/HER3 Y1289 | 1.558 | 0.02449 |
| HSP90a T5/7 | 1.555 | 0.02516 |
| Bak | 1.254 | 0.02534 |
| EGFR Y992 | 1.970 | 0.02669 |
| Met Y1234/1235 | 1.556 | 0.02747 |
| p53 | 1.390 | 0.02815 |
| ErbB3/HER3 Y1197 | 1.363 | 0.02834 |
| c-Abl T735 | 1.596 | 0.02950 |
| SGK1 S78 | 1.510 | 0.03094 |
| E-Cadherin | 1.903 | 0.03501 |
| p70 S6 Kinase S371 | 1.621 | 0.03502 |
| PTEN | 1.334 | 0.03623 |
| AMPKalpha S485 | 2.359 | 0.04484 |
| Bcl_xL | 1.275 | 0.04664 |
| Ros Y2274 | 1.481 | 0.04682 |
| EGFR Y1148 | 1.407 | 0.05060 |
| Ras_GRF1 | 1.237 | 0.05305 |
| Musashi | 1.320 | 0.05571 |
| MEK 1/2 S217/221 | 1.772 | 0.05644 |
| Jak2 Y1007 | 1.647 | 0.05981 |
| mTOR | 0.679 | 0.06052 |
| BCL-2 | 1.315 | 0.06162 |
| Cyclin A | 1.513 | 0.06673 |
| Cl Caspase 3 D175 | 1.484 | 0.06774 |
| c-Met TOTAL | 1.220 | 0.06811 |
| Aurora A T288/B T232/C T198 | 1.651 | 0.07378 |
| IL6 | 1.192 | 0.07693 |
| HSP70 | 1.429 | 0.08222 |
| ATM S1981 | 1.438 | 0.09109 |
| TGF-beta | 1.188 | 0.09217 |
| RAS TOTAL | 1.486 | 0.09305 |
| PAK1 S199/204-PAK2 S192/197 | 1.852 | 0.09472 |
| p70 S6 Kinase T389 | 2.148 | 0.09631 |
| Ret Y905 | 1.885 | 0.10194 |
| FAK Y576/577 | 1.291 | 0.10519 |
| Elk S383 | 1.926 | 0.10553 |
| LIMK1 T508/LIMK2 T505 | 1.351 | 0.10813 |
| Cyclin B1 V152 | 1.294 | 0.11916 |
| XIAP | 1.311 | 0.11964 |
| BAD S136 | 1.550 | 0.12064 |
| Insulin Receptor Beta | 1.219 | 0.12478 |
| PDGFR alpha Y754 | 1.266 | 0.12540 |
| MCSFR Y723 | 1.215 | 0.12636 |
| Cyclin D1 T286 | 1.693 | 0.13377 |
| c-Raf S338 | 1.229 | 0.14714 |
| Tuberin/TSC2 Y1571 | 1.417 | 0.15390 |
| ALK Y1604 | 1.302 | 0.15741 |
| IGFBP7 | 1.180 | 0.16058 |
| ER alpha S118 | 1.293 | 0.16417 |
| PKM2 total* | 1.339 | 0.17288 |
| PAK1 T423/PAK2 T402 | 1.245 | 0.17360 |
| Shc Y317 | 1.307 | 0.17446 |
| Stat3 Y705 | 1.421 | 0.17478 |
| EGFR | 1.184 | 0.18126 |
| mTOR S2448 | 1.515 | 0.18490 |
| A-Raf S299 | 1.335 | 0.18618 |
| B-Raf S445 | 1.392 | 0.18641 |
| IGF-1R Y1131/IR Y1146 | 1.596 | 0.20059 |
| LKB1 S428 | 1.652 | 0.20214 |
| PTEN S380 | 1.440 | 0.20405 |
| IkappaB-alpha S32/36 | 1.317 | 0.20641 |
| VEGFR2 Y996 | 1.205 | 0.20760 |
| B-RAF | 1.341 | 0.21364 |
| PLK1 T210 | 1.301 | 0.21787 |
| ER TOTAL | 1.304 | 0.22013 |
| p27 Kip1 | 1.253 | 0.22567 |
| PKM2 Y105* | 1.403 | 0.22613 |
| MARCKS S152/156 | 1.421 | 0.23094 |
| c-Kit Y703 | 1.408 | 0.23440 |
| TNF alpha | 1.119 | 0.23559 |
| PDL1 | 0.548 | 0.24967 |
| SAPK/JNK T183/Y185 | 1.410 | 0.25293 |
| ATR S428 | 1.345 | 0.26032 |
| ErbB2/HER2 Y877 | 1.283 | 0.27572 |
| Rb S780 | 1.516 | 0.27903 |
| AKT S473 | 1.795 | 0.29461 |
| Beclin 1 | 1.235 | 0.29806 |
| Bax | 1.132 | 0.30553 |
| ErbB3/HER3 | 0.828 | 0.31463 |
| p53 S15 | 1.586 | 0.31482 |
| BIM | 1.211 | 0.31840 |
| FOXO1 S256 | 1.338 | 0.32018 |
| AXL | 1.199 | 0.32147 |
| Heregulin | 0.654 | 0.32482 |
| eIF4G S1108 | 1.471 | 0.32846 |
| p70 S6 Kinase T412 | 1.352 | 0.33362 |
| BAD S112 | 1.355 | 0.33540 |
| Cofilin S3 | 1.783 | 0.34545 |
| S6 Ribosomal Protein S240/244 | 0.457 | 0.35570 |
| SMAD2 S245/250/255 | 1.425 | 0.36071 |
| IGF1R Y1135/36-IR Y1150/51 | 1.118 | 0.36682 |
| c-Abl Y245 | 1.215 | 0.36882 |
| p27 T187 | 1.152 | 0.37786 |
| c-ErbB2/HER2 | 1.184 | 0.38228 |
| p90RSK T359/S363 | 1.248 | 0.38496 |
| p70S6 Kinase | 1.193 | 0.38956 |
| Src Y527 | 1.356 | 0.39408 |
| Cyclin D1 (G124-326) | 1.272 | 0.39879 |
| STAT3 S727 | 1.302 | 0.43724 |
| Histone H3 S28 | 1.222 | 0.45793 |
| GSK-3alpha/beta S21/9 | 1.294 | 0.46485 |
| Src family Y416 | 0.804 | 0.46735 |
| c-Myc | 1.078 | 0.46892 |
| PKC delta/theta S643/S676 | 1.447 | 0.47243 |
| CrkL Y207 | 1.186 | 0.47357 |
| AMPK total* | 0.555 | 0.48571 |
| BAD | 1.198 | 0.49606 |
| Acetyl-CoA Carboxylase S79 | 1.295 | 0.52475 |
| ERK 1-2 T202/Y204 | 1.337 | 0.52561 |
| LC3B | 1.109 | 0.52742 |
| ATP CItrate Lyase S454 | 1.229 | 0.55317 |
| 4EBP1 T70 | 1.117 | 0.57516 |
| Stat 1 Y701 | 1.092 | 0.63519 |
| 4EBP1 S65 | 1.143 | 0.64049 |
| PDGFR beta | 1.147 | 0.64381 |
| HSP27 S82 | 0.821 | 0.66231 |
| Cleaved PARP D214 | 1.223 | 0.68060 |
| MDM2 S166 | 1.189 | 0.68299 |
| Actin beta | 1.000 | 0.69050 |
| 4EBP1 T37/46 | 0.828 | 0.69462 |
| CREB S133 | 1.164 | 0.70658 |
| PDK1 S241 | 1.118 | 0.71192 |
| AMPKbeta S108 | 1.081 | 0.72119 |
| HIF-1 alpha | 1.096 | 0.74602 |
| Stat 5 Y694 | 0.929 | 0.76999 |
| KI67 | 1.120 | 0.78810 |
| p38 MAP Kinase T180/Y182 | 1.075 | 0.85379 |
| PKC zeta/lambda T410/403 | 0.971 | 0.85427 |
| ATF2 T69/71 | 0.959 | 0.87575 |
| PDGFR beta Y716 | 0.981 | 0.89981 |
| Vimentin | 1.021 | 0.94613 |
| FKHR T24/FKHRL1 T32 | 1.039 | 0.94659 |
| pYAP S127 | 1.022 | 0.94948 |
| GRB2 | 1.013 | 0.95614 |
| IRS1 | 0.996 | 0.98436 |
| p90RSK S380 | No Data | No Data |
| Smad1 Ser463/465-Smad5 Ser463/ | No Data | No Data |
| 465-Smad9 Ser465/467 | | |
| FADD S194 | No Data | No Data |
| Chk1 S345 | No Data | No Data |
| eNOS/NOS III S116 | No Data | No Data |
| Histone H3 S10 | No Data | No Data |
| IRS-1 S612 | No Data | No Data |
| PKA C T197 | No Data | No Data |
| PUMA | No Data | No Data |

Results were normalized using a β-actin probe antibody.
*Indicates tested subsequently in a separate hypothesis driven RPPA analysis of the same tumor specimens and normalized to a separate β actin probe.

### Example 2

### Use of CYP Gene Amplification to Direct Therapeutic Intervention in Cancer

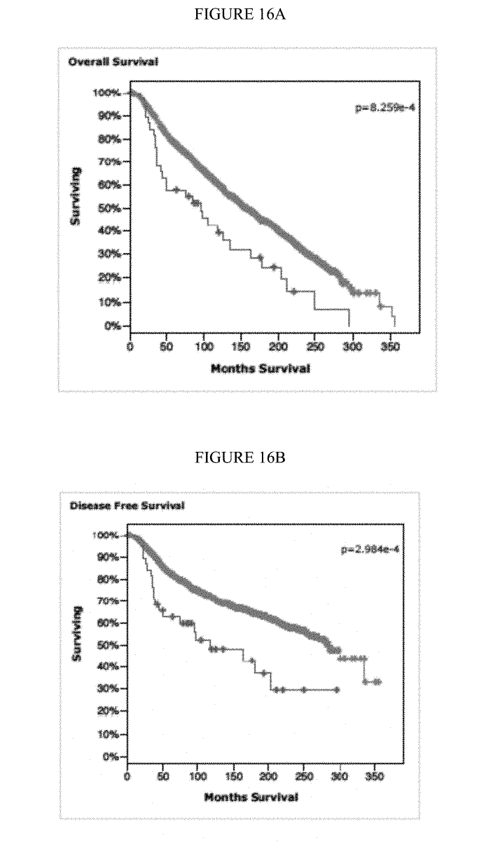

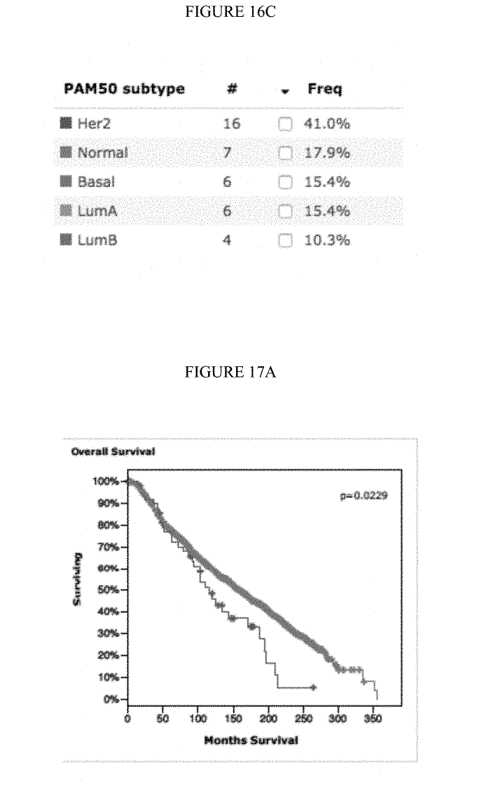

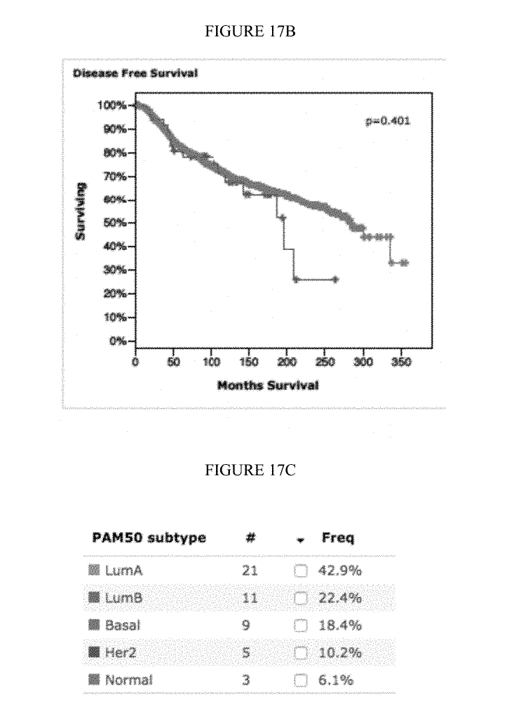

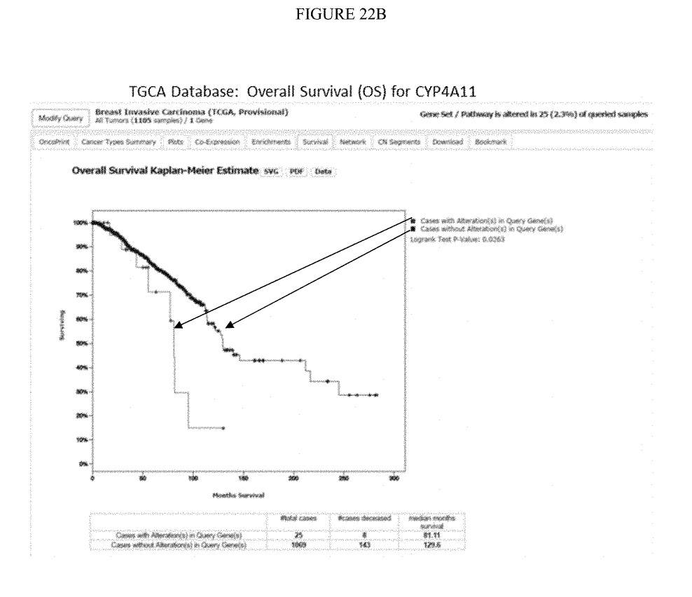

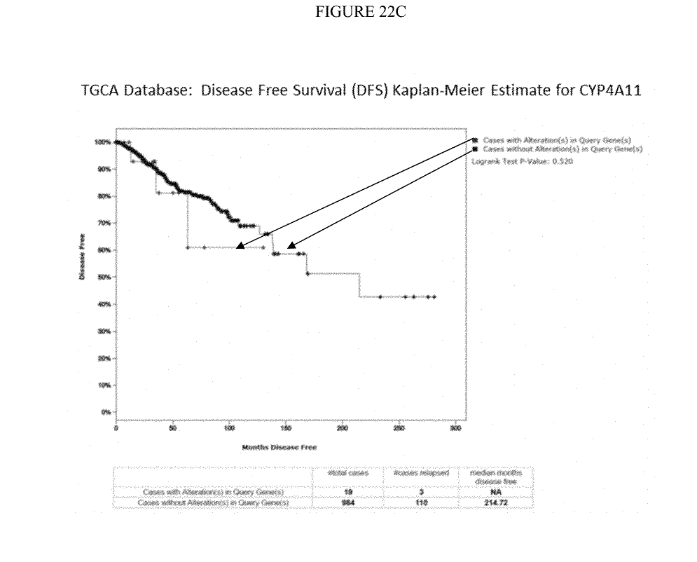

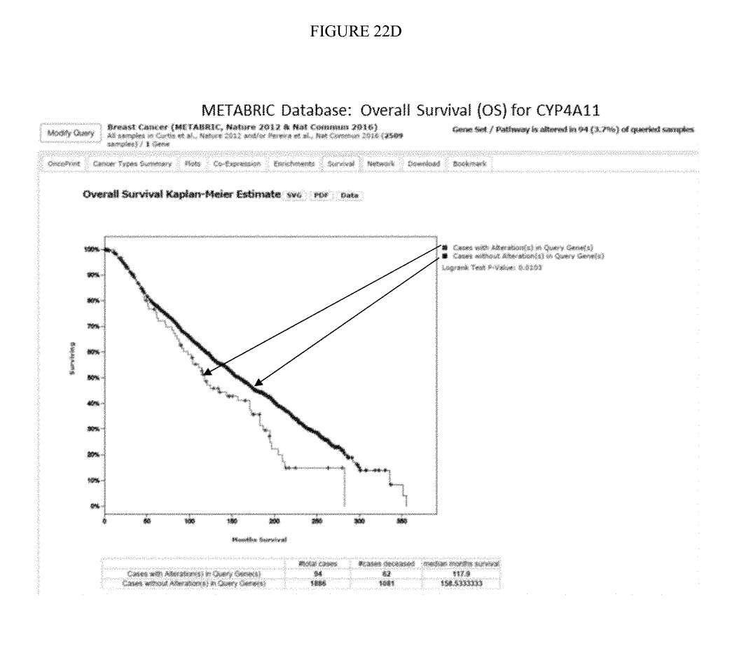

CYP monooxygenases play unexpected roles in cancer progression, but validation of cancer cell intrinsic CYPs as therapeutic targets in breast cancer remains unproven and therapeutically effective inhibitors have yet to be developed. Little is known about the cancer cell intrinsic roles of the cytochrome P450 monooxygenase/epoxygenase enzymes, such as CYPs 2J2, 3A4 and 4A11 and the roles of their epoxyeicosatrienoic acid (EET) products in breast cancer progression. As described herein, it has been discovered that CYP3A4 exhibits robust epoxygenase activity and is required for tumorigenicity of the ER+HER2− breast cancer cell line MCF-7, which exhibits CYP3A4 amplification. Unexpectedly, it has been discovered that the mechanism of metformin inhibition of breast cancer is due, in part, to disruption of CYP-mediated EETs biosynthesis. This mechanism of metformin action is supported by a metformin-CYP3A4 co-crystal and spectroscopic demonstration of metformin binding to CYP3A4 nanodiscs. Using CYP nanodiscs, it has been demonstrated that metformin binds to CYP2C8 and CYP2J2, which have been linked to cancer progression. What is not known is which breast cancers may be sensitive to biguanides. CYPs can be up-regulated in breast cancer by many mechanisms, including mRNA up-regulation, which is mutually exclusive between CYPs in each breast tumor (METABRIC and TGCA databases). As described herein, RNA profiling by U133 chip analysis may be used in existing METABRIC and TGCA-provisional breast cancer databases to further develop signatures that can be detected from formalin fixed paraffin embedded tissue or fresh biopsy specimens. The overall survival (OS) and disease free survival (DFS) associations of gene expression of the CYP monooxygenases CYP1A1, CYP2C8/9, CYP2J2, CYP3A4/5, CYP4F2/3/11, and CYP4A11 were profiled. The up-regulation of mRNA was profiled individually and then in pairs, first in METABRIC which is the largest database, and then in TGCA-provisional. The few duplicate specimens were deleted. The two CYPs that were individually significant were CYP2J2 (FIG. 16) and CYP4A11 (FIG. 17; see also, FIG. 22) and they were associated with the full range of breast cancer subtypes but with differing trends.

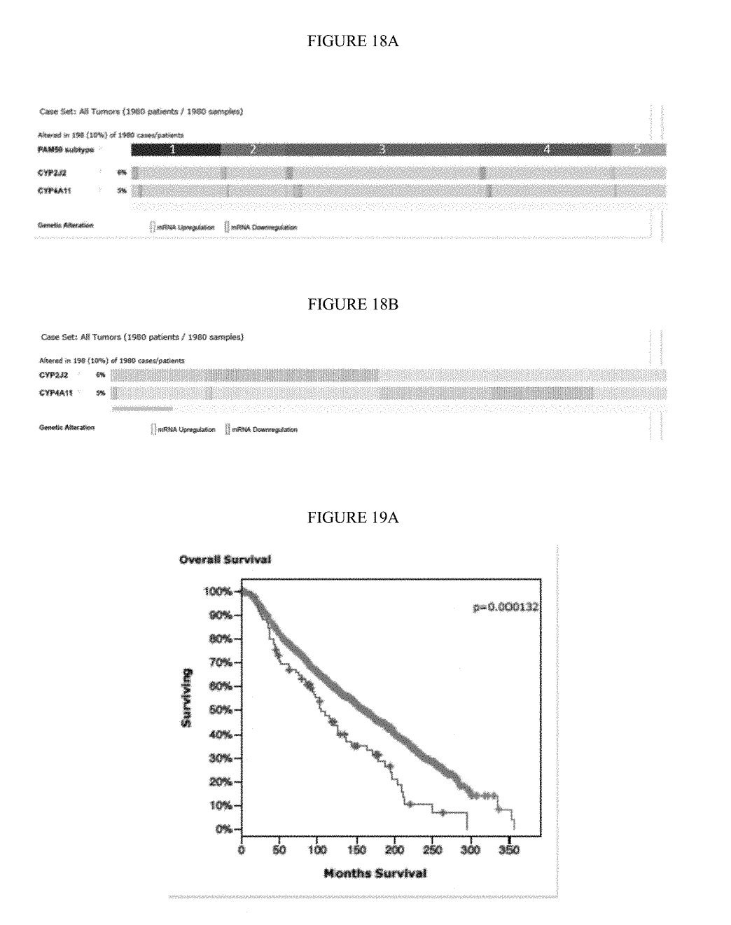

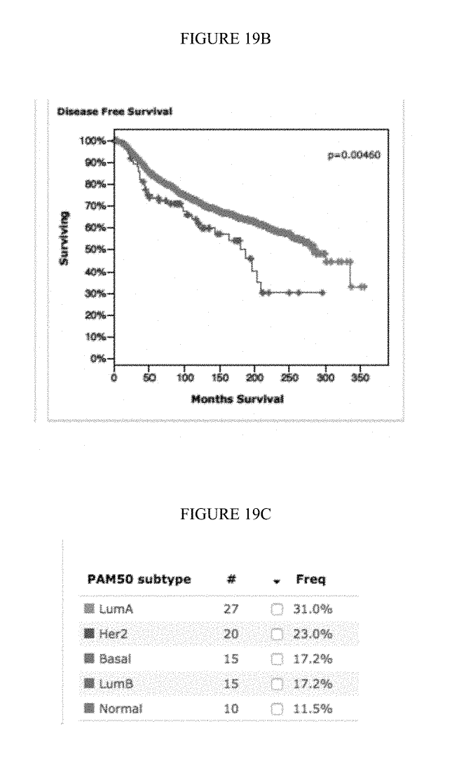

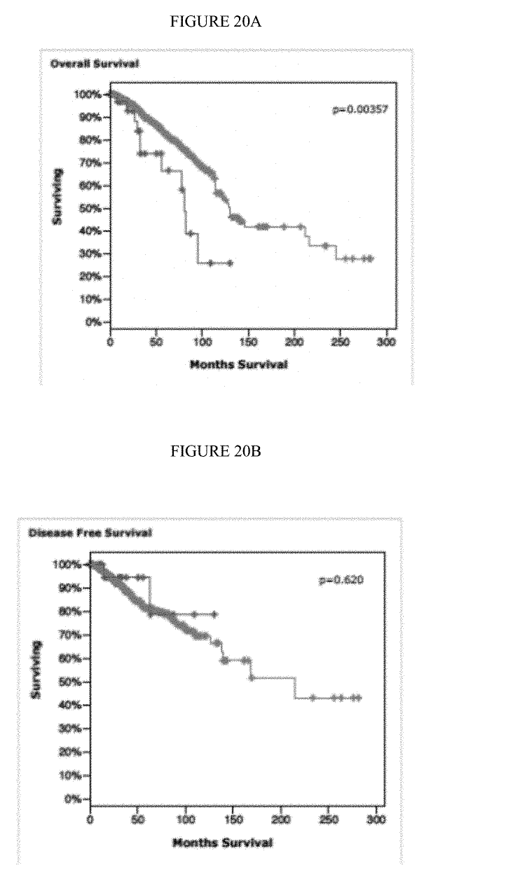

PAM50 profiling reveals expression of CYP2J2 predominantly in HER2+ and basal or normal breast cancer (FIG. 16C), while CYP4A11 is associated with luminal A/B breast cancer (FIG. 17C). CYP2J2 is the most significant CYP for survival of 10 CYPs profiled and its mRNA up-regulation is associated with decreased OS; 50% at 7.5 years (P=8.26e-4). Up-regulation of CYP4A11 mRNA is associated with luminal A/B breast cancer and is also associated with decreased OS; 50% at 9 years (P=0.0229). What is also striking about the DFS pattern is that consistent with known clinical recurrence patterns, with CYP2J2 being associated with HER2+ and basal/normal breast cancer and early recurrence (FIG. 16B), while CYP4A11, associated with luminal breast cancer, is associated with later recurrences (FIG. 17B). While expression of CYP2J2 and CYP4A11 trends with different PAM50 breast cancer subtypes (FIG. 18A) is nearly mutually exclusive (FIG. 18B), a combined predictive tool can be obtained by combining these genes (FIG. 19). Using the combined predictive tool, the presence of either CYP2J2 or CYP4A11 upregulation is associated with decreased OS (P=0.000132) and DFS (P=0.00460) in 4.4% of breast cancer in the METABRIC study of 1980 patients. When assessed in the TGCA-provisional data set, CYP2J2 exhibits a trend toward decreased OS but this result is not significant. In contrast, CYP4A11 significant association with decreased OS (P=0.00357) (FIG. 20; see also, FIG. 22).

Because personalized medicine approaches will allow us to identify patients with CYP monooxygenase up-regulation at the gene expression or protein levels, there is a therapeutic opportunity and gap in knowledge that needs to be filled by revealing which CYPs promote mammary cancer progression and which are responsive to metformin. Our hypothesis is that CYP monooxygenases are susceptible to inhibition of arachidonic acid (AA) epoxidation to epoxyeicosatrienoic acids (EETs) which promote breast cancer proliferation, survival and clonogenicity (Mitra et al., J Biol Chem. 2011 May 20; 286(20): 17543-59; Guo et al., ER+ Breast Tumor Growth is Dependent on CYP3A4 Epoxygenase Activity and Directly Suppressed by a Highly Potent Metformin-Derived Heme Binding Inhibitor. In review).

As described herein, it is proposed to perform a Nanostring or multiplexed quantitative PCR assay of the CYP2J2 and CYP4A11 genes from formalin fixed paraffin embedded breast cancer tissue. Expression can then be normalized to genes GAPDH, β-actin, RPLPO, GUS and TFRC similar to the Oncotype Dx, and use a Cox regression model to correlated score with DFS and or OS. It is proposed that this assay will be prognostic of DFS and OS, as well as predictive of breast cancer response to biguanide drugs including metformin, phenformin, buformin, and hexyl-benzyl-biguanide.

### Example 3

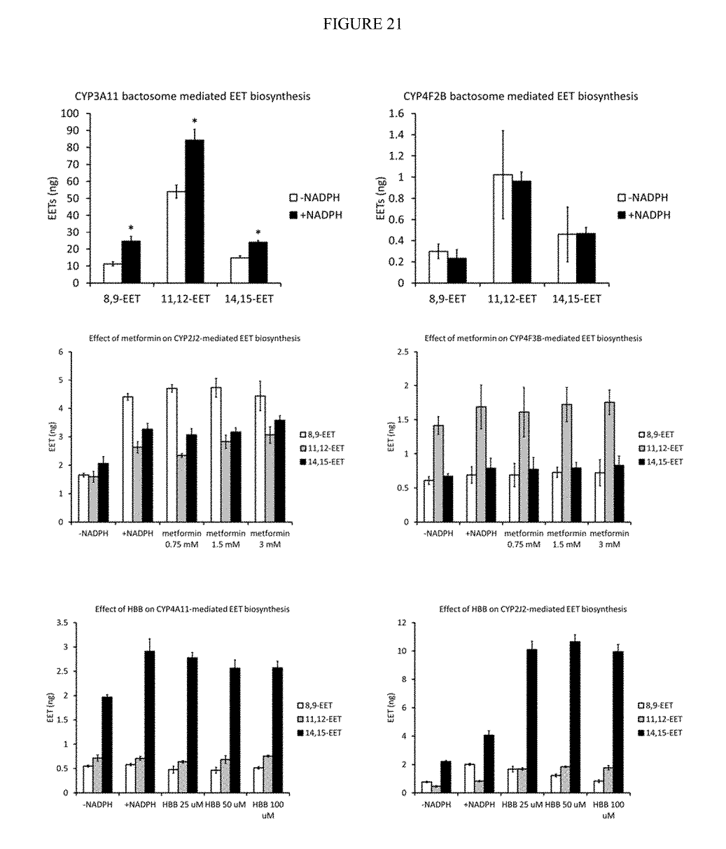

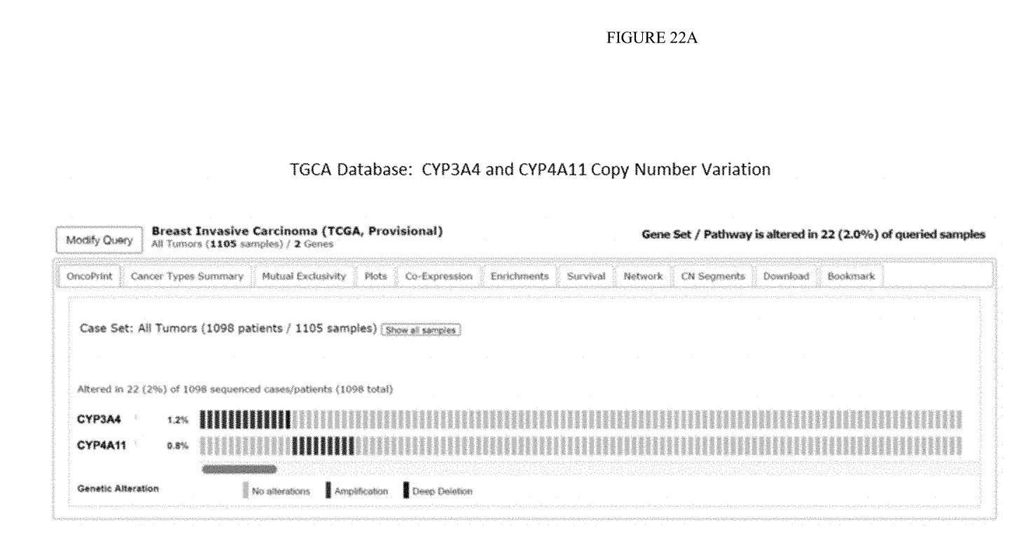

FIG. 21 shows CYP3A11 and CYP4F2B bactosome mediated EET biosynthesis (top, left and right panels). Additionally, FIG. 21 shows the effect of metformin and HBB on CYP2J2-, CYP4F3B- and CYP4A11-mediated EET biosynthesis (middle and bottom panels).

### Example 4

CYP4A11 was previously thought to be the main omega hydroxylase and was not known to be an arachidonic acid epoxygenase. The working hypothesis in the literature is that CYP4A11 is a 20 hydroxylase for arachidonic acid, promoting tumor growth through that mechanism. However, as described herein, it is proposed that that alteration of CYP4A11 mRNA correlates with decreased overall survival in the METABRIC (2059 patients) and TGCA provisional (1105 patients) databases (see, FIGS. 22A-24E). It is also proposed herein that increase of CYP4A11 mRNA correlates with decreased overall survival in both data bases. In order to confirm this correlation, a z score of ±2.0 for the METABRIC database and a z score of ±1.5 for the TGCA database was used, which has half as many patients.

### Methods

In cBioPortal, either METABRIC or Breast Cancer TGCA provisional was chosen.

### For METABRIC:

METABRIC 2059 samples; and mRNA expression Z scores by Illumina v 3 microarray Z score threshold=±2.0

Obtain an Oncoprint profile which is a colored profile of all patients with up or down regulated CYP4A11 mRNA. Then ask about overall survival and disease free survival. To isolate the up-regulated patients, obtain the sample ID's in sequence and then test this list using the “query this study” which generates an overall survival plot.

For METABRIC the results for CYP4A11 are significant with the more stringent z score±2.0. Note that this study has twice as many patients as TGCA 2059 vs. 1105 (see, e.g., FIG. 24).

### For TGCA Provisional:

TGCA Provisional 1105 Samples; mRNA Z Scores±2.0 or z Scores±1.5

Obtain an Oncoprint profile, which is a colored profile of all patients with up or down regulated CYP4A11 mRNA. Then ask about overall survival and disease free survival. To isolate the up-regulated patients, obtain the patient ID's in sequence and then test this list using the “query this study” which generates an overall survival plot.

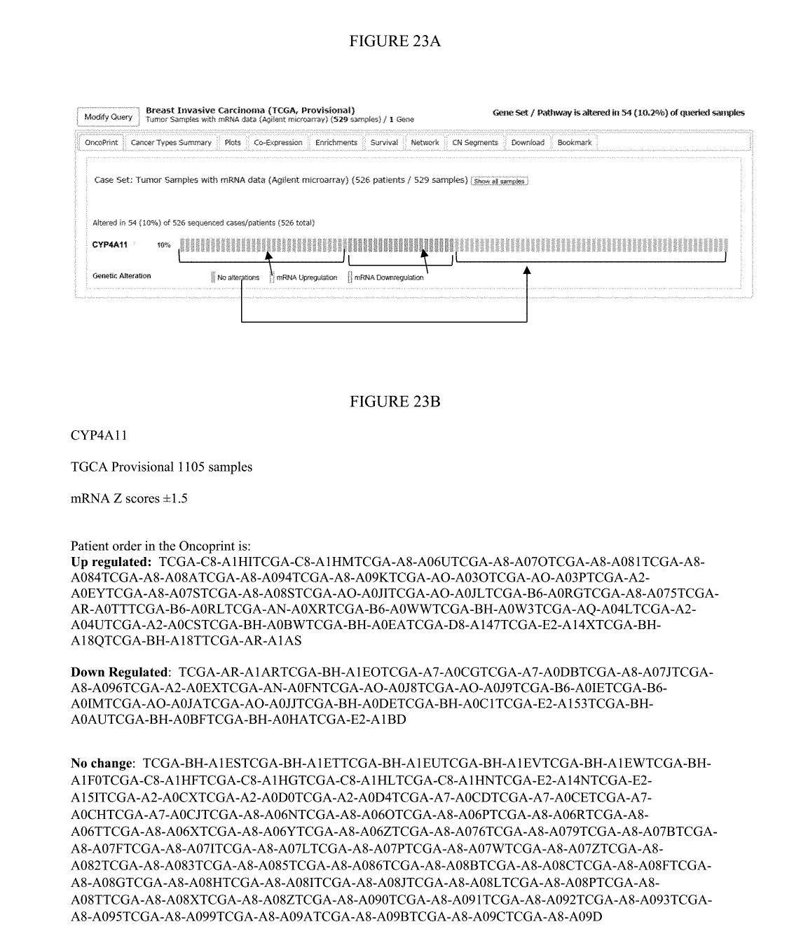

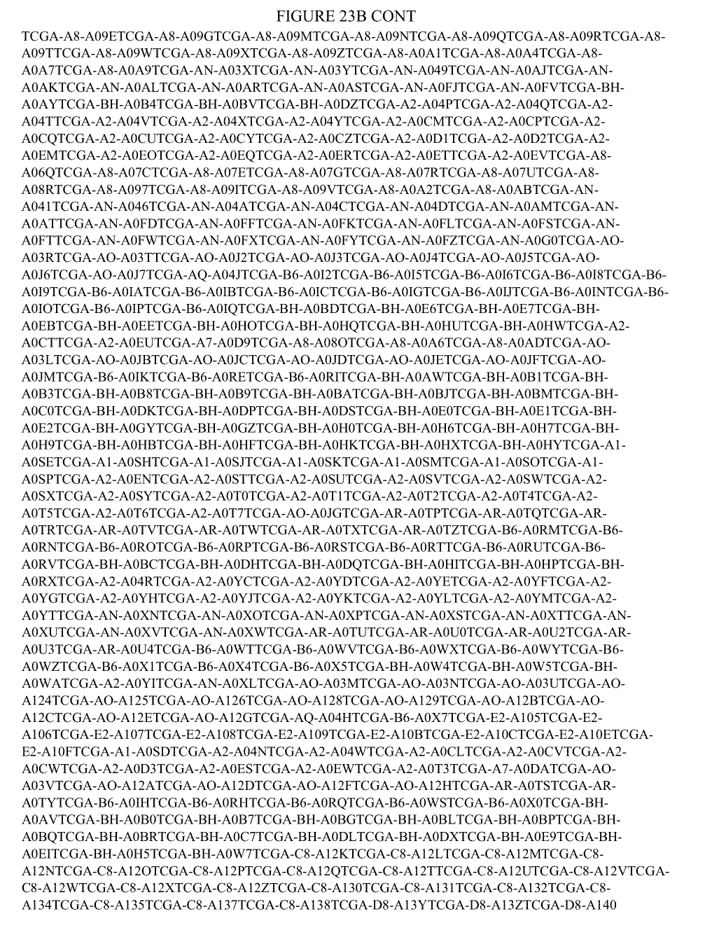

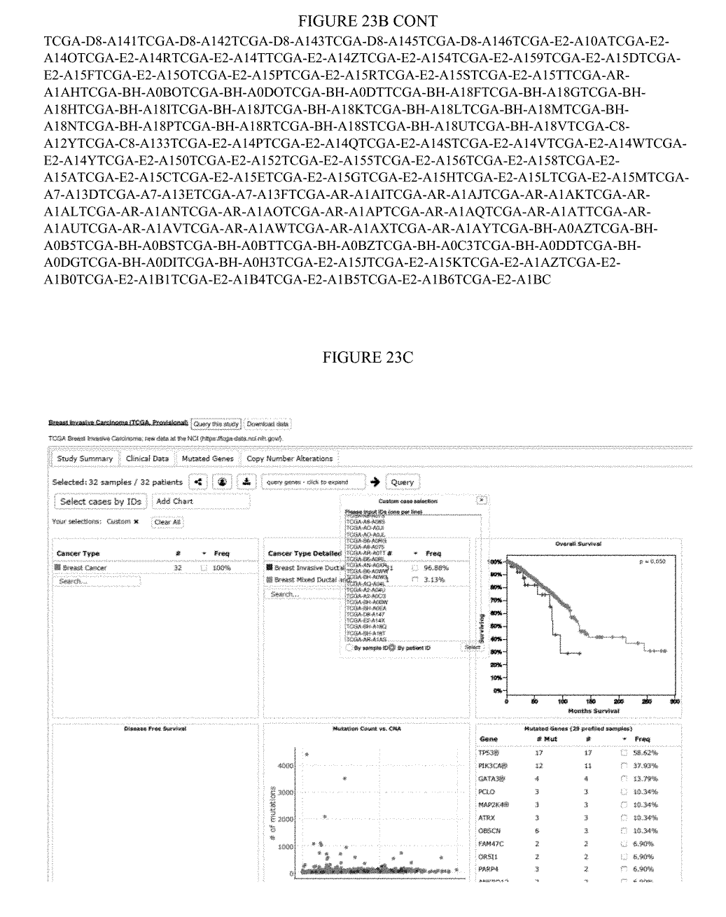

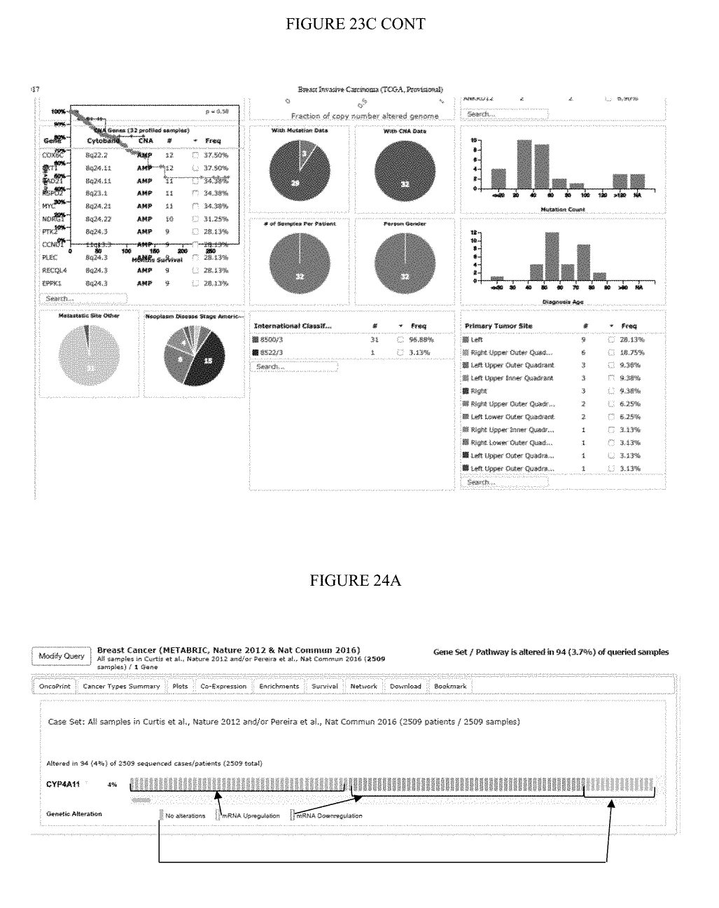

For TGCA provisional the results for CYP4A11 are significant with the less stringent z scores±1.5. Significance is reached with either CYP4A11 up-regulated vs. not altered or CYP4A11 up or down regulated vs. not altered (see, e.g., FIG. 23).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for identifying a patient having cancer that can be treated with a biguanide compound or other CYP epoxygenase inhibitor, comprising: 1) obtaining a cancer cell sample from the patient; and 2) detecting whether expression of at least one CYP is increased and/or whether expression of soluble epoxide hydrolase (EPHX2) is decreased in a cancer cell from the sample, by measuring the expression level of the at least one CYP and/or EPHX2; and 3) identifying the patient having cancer as being treatable with a biagunide compound or other CYP epoxygenase inhibitor when increased expression of at least one CYP and/or decreased expression of EPHX2 is detected, as compared to a control.

2. The method of claim 1, comprising detecting increased expression of at least one CYP.

3. The method of claim 1, wherein the at least one CYP is CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5A1, CYP7A1, CYP7B1, CYP8A1, CYP8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1 and/or CYP51A1.

4. The method of claim 1, comprising detecting decreased expression of EPHX2.

5. The method of claim 1, wherein the cancer is a solid tumor cancer selected from the group consisting of breast, ovarian, endometrial/uterine, bladder, glioma and lung adenocarcinoma cancer.

6. The method of claim 1, wherein the biguanide compound comprises structural group:

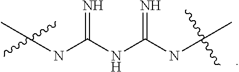 [SMILES: *NC(=N)NC(=N)N*]

7. The method of claim 1, wherein the biguanide compound is a compound of formula I: 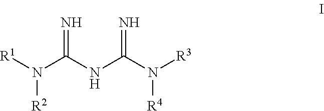
wherein:
R¹ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl or —O(C₂-C₁₂)alkynyl of R¹ is optionally substituted with one or more Z^{1a} groups and wherein any (C₃-C₈)carbocycle, 5-10 membered heteroaryl or aryl of R¹ is optionally substituted with one or more Z^{1b} groups;
R² is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl of R² is optionally substituted with one or more Z^{2a} groups and wherein any (C₃-C₈)carbocycle, 5-10 membered heteroaryl or aryl of R¹ is optionally substituted with one or more Z^{2b} groups;
R³ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl or —O(C₂-C₁₂)alkynyl of R³ is optionally substituted with one or more Z^{3a} groups and wherein any (C₃-C₈)carbocycle, 5-10 membered heteroaryl or aryl of R³ is optionally substituted with one or more Z^{3b} groups;
R⁴ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl of R⁴ is optionally substituted with one or more Z^{4a} groups and wherein any (C₃-C₈)carbocycle, 5-10 membered heteroaryl or aryl of R⁴ is optionally substituted with one or more Z^{4b} groups;
Z^{1a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{1a} is optionally substituted with one or more groups selected from (C₁-C₆)alkyl, —OH, halogen and —O(C₁-C₆)alkyl;
Z^{1b} is (C₁-C₆)alkyl, —OH, halogen or —O(C₁-C₆)alkyl;
Z^{2a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{2a} is optionally substituted with one or more groups selected from (C₁-C₆)alkyl, —OH, halogen and —O(C₁-C₆)alkyl;
Z^{2b} is (C₁-C₆)alkyl, —OH, halogen or —O(C₁-C₆)alkyl;
Z^{3a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z³a is optionally substituted with one or more groups selected from (C₁-C₆)alkyl, —OH, halogen and —O(C₁-C₆)alkyl;
Z^{3b} is (C₁-C₆)alkyl, —OH, halogen or —O(C₁-C₆)alkyl;
Z^{4a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{4a} is optionally substituted with one or more groups selected from (C₁-C₆)alkyl, —OH, halogen and —O(C₁-C₆)alkyl; and
Z^{4b} is (C₁-C₆)alkyl, —OH, halogen or —O(C₁-C₆)alkyl;
or a pharmaceutically acceptable salt thereof.

8. The method of claim 7, wherein the compound of formula I is: 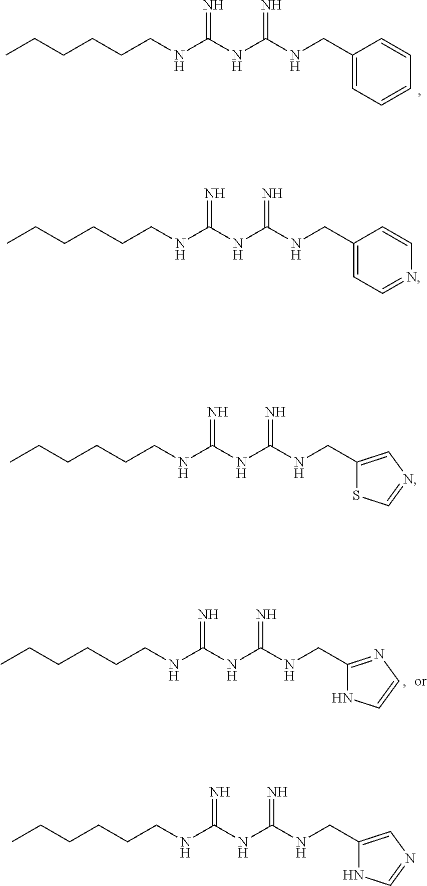 [SMILES: CCCCCCNC(=N)NC(=N)NCc1ccccc1 ; CCCCCCNC(=N)NC(=N)NCc1ccncc1 ; CCCCCCNC(=N)NC(=N)NCc1cncs1 ; CCCCCCNC(=N)NC(=N)NCc1ncc[nH]1 ; CCCCCCNC(=N)NC(=N)NCc1cnc[nH]1]
or a pharmaceutically acceptable salt thereof.

9. The method of claim 1, wherein the biguanide compound is selected from the group consisting of: 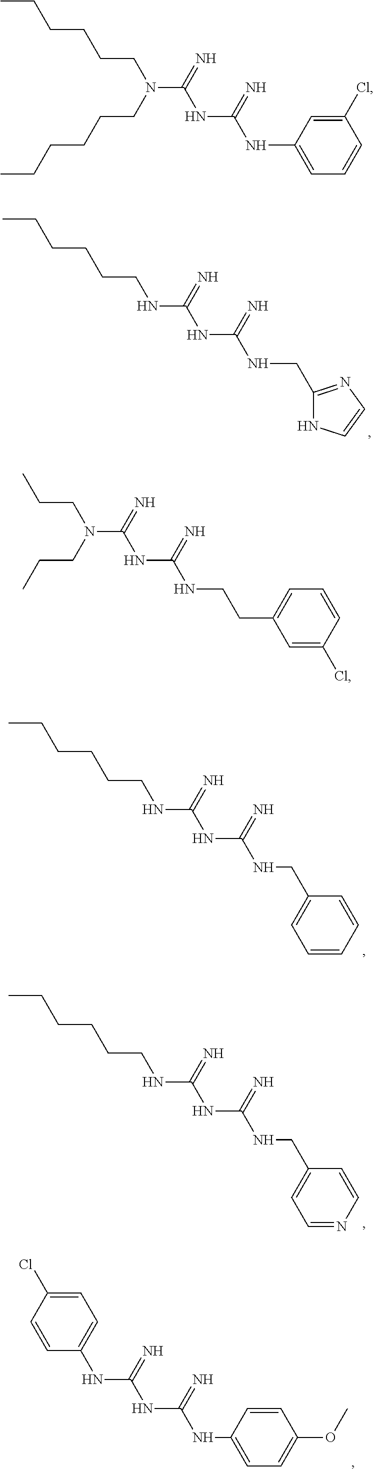 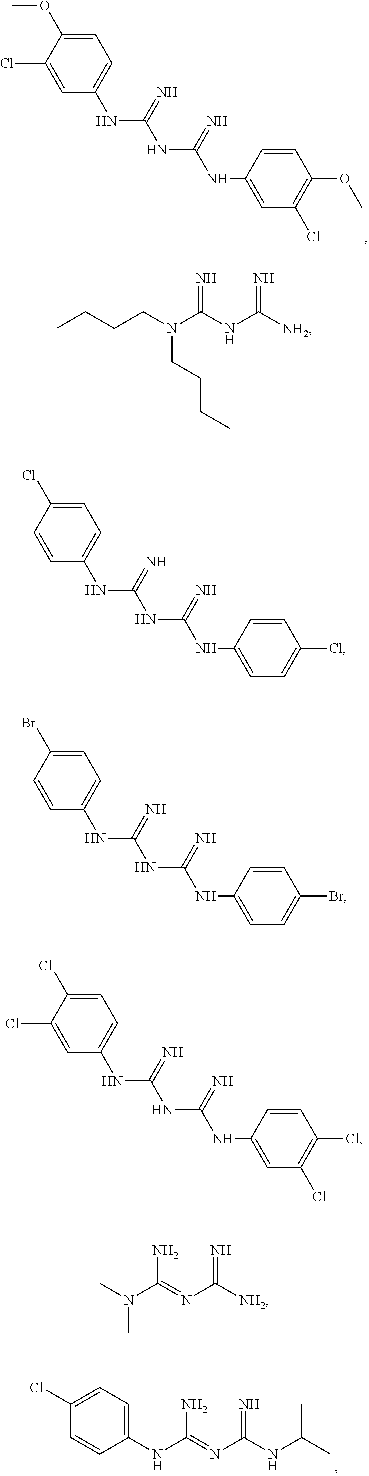 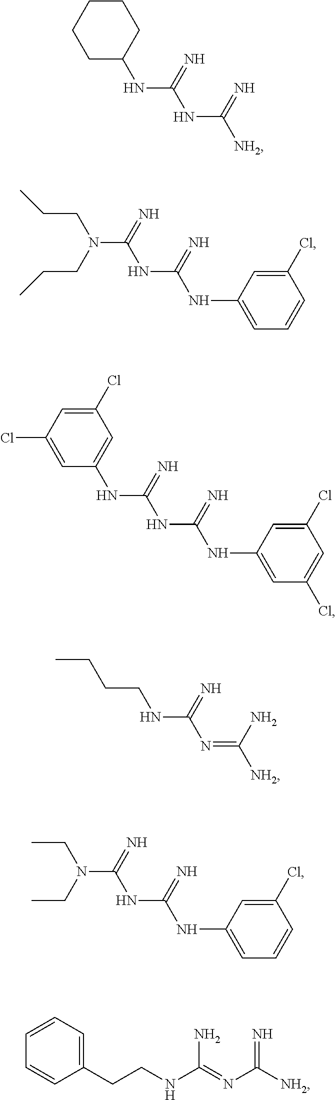 [SMILES: N=C(N)NC(=N)NC1CCCCC1 ; CCCN(CCC)C(=N)NC(=N)Nc1cccc(Cl)c1 ; N=C(NC(=N)Nc1cc(Cl)cc(Cl)c1)Nc1cc(Cl)cc(Cl)c1 ; CCCCNC(=N)N=C(N)N ; CCN(CC)C(=N)NC(=N)Nc1cccc(Cl)c1 ; N=C(N)/N=C(\N)NCCc1ccccc1]
and pharmaceutically acceptable salts thereof.

10. The method of claim 1, further comprising 4) administering an effective amount of a biguanide compound or other CYP epoxygenase inhibitor to the identified patient.

11. A method for treating cancer in a patient comprising administering an effective amount of a biguanide compound or other CYP epoxygenase inhibitor to the patient, wherein the cancer was determined to comprise increased expression of at least one CYP and/or decreased expression of soluble epoxide hydrolase (EPHX2).

12. The method of claim 11, wherein the cancer was determined to comprise increased expression of at least one CYP.

13. The method of claim 11, wherein the at least one CYP gene is CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5A1, CYP7A1, CYP7B1, CYP8A1, CYP8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1 and/or CYP51A1.

14. The method of any one of claim 11, wherein the cancer was determined to comprise decreased expression of EPHX2.

15. The method of claim 11, wherein the cancer is a solid tumor cancer selected from the group consisting of breast, ovarian, endometrial/uterine, bladder, glioma and lung adenocarcinoma cancer.

16. The method of claim 11, wherein the biguanide compound comprises structural group:

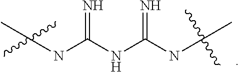 [SMILES: *NC(=N)NC(=N)N*]

17. The method of claim 11, wherein the biguanide compound is a compound of formula I: 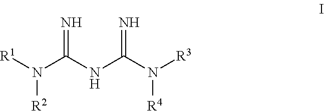
wherein:
R¹ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl or —O(C₂-C₁₂)alkynyl of R¹ is optionally substituted with one or more Z^{1a} groups and wherein any (C₃-C₈)carbocycle, 5-10 membered heteroaryl or aryl of R¹ is optionally substituted with one or more Z^{1b} groups;
R² is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl of R² is optionally substituted with one or more Z^{2a} groups and wherein any (C₃-C₈)carbocycle, 5-10 membered heteroaryl or aryl of R¹ is optionally substituted with one or more Z^{2b} groups;
R³ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl or —O(C₂-C₁₂)alkynyl of R³ is optionally substituted with one or more Z^{3a} groups and wherein any (C₃-C₈)carbocycle, 5-10 membered heteroaryl or aryl of R³ is optionally substituted with one or more Z^{3b} groups;
R⁴ is H, (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl, —OH, (C₃-C₈)carbocycle, 5-10 membered heteroaryl or aryl, wherein any (C₁-C₁₂)alkyl, (C₂-C₁₂)alkenyl, (C₂-C₁₂)alkynyl, —O(C₁-C₁₂)alkyl, —O(C₂-C₁₂)alkenyl, —O(C₂-C₁₂)alkynyl of R⁴ is optionally substituted with one or more Z^{4a} groups and wherein any (C₃-C₈)carbocycle, 5-10 membered heteroaryl or aryl of R⁴ is optionally substituted with one or more Z^{4b} groups;
Z^{1a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{1a} is optionally substituted with one or more groups selected from (C₁-C₆)alkyl, —OH, halogen and —O(C₁-C₆)alkyl;
Z^{1b} is (C₁-C₆)alkyl, —OH, halogen or —O(C₁-C₆)alkyl;
Z^{2a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z²a is optionally substituted with one or more groups selected from (C₁-C₆)alkyl, —OH, halogen and —O(C₁-C₆)alkyl;
Z^{2b} is (C₁-C₆)alkyl, —OH, halogen or —O(C₁-C₆)alkyl;
Z^{3a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z³a is optionally substituted with one or more groups selected from (C₁-C₆)alkyl, —OH, halogen and —O(C₁-C₆)alkyl;
Z^{3b} is (C₁-C₆)alkyl, —OH, halogen or —O(C₁-C₆)alkyl;
Z^{4a} is —OH, halogen, —O(C₁-C₆)alkyl, —C(═O)O(C₁-C₆)alkyl, (C₃-C₈)carbocycle, 5-10 membered heteroaryl or aryl, wherein any (C₃-C₈)carbocycle, 5-10 membered heteroaryl or aryl of Z^{4a} is optionally substituted with one or more groups selected from (C₁-C₆)alkyl, —OH, halogen and —O(C₁-C₆)alkyl; and
Z^{4b} is (C₁-C₆)alkyl, —OH, halogen or —O(C₁-C₆)alkyl;
or a pharmaceutically acceptable salt thereof.

18. The method of claim 17, wherein the compound of formula I is: 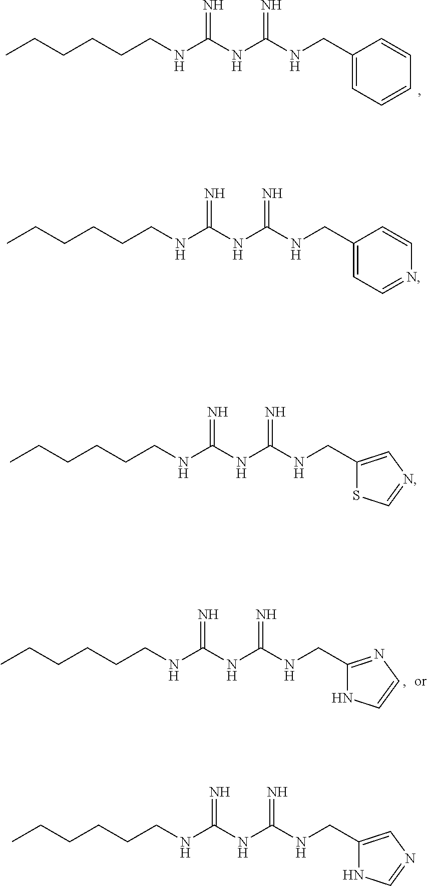 [SMILES: CCCCCCNC(=N)NC(=N)NCc1ccccc1 ; CCCCCCNC(=N)NC(=N)NCc1ccncc1 ; CCCCCCNC(=N)NC(=N)NCc1cncs1 ; CCCCCCNC(=N)NC(=N)NCc1ncc[nH]1 ; CCCCCCNC(=N)NC(=N)NCc1cnc[nH]1]
or a pharmaceutically acceptable salt thereof.

19. The method of claim 11, wherein the biguanide compound is selected from the group consisting of: 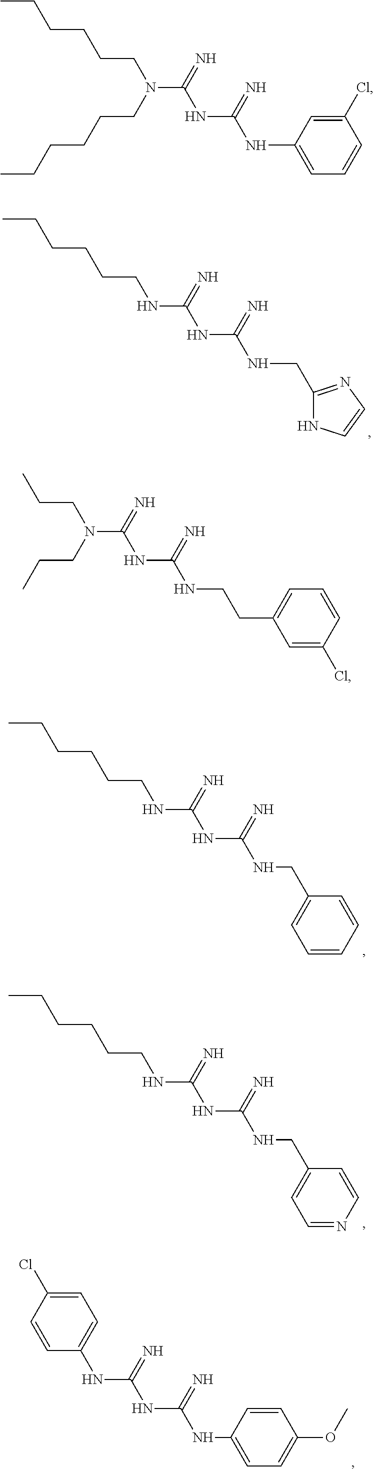 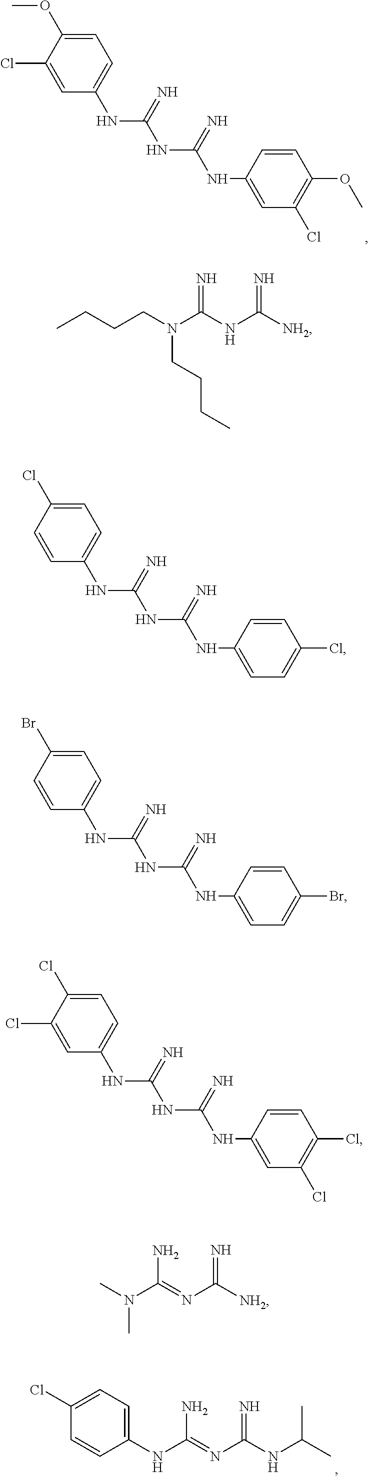 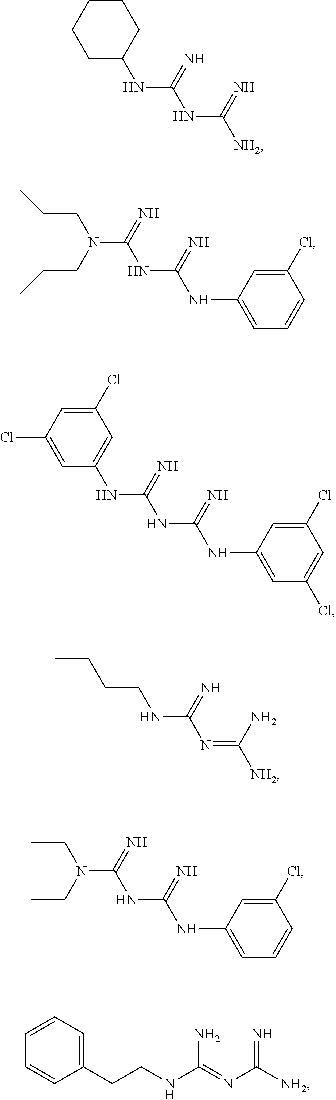 [SMILES: N=C(N)NC(=N)NC1CCCCC1 ; CCCN(CCC)C(=N)NC(=N)Nc1cccc(Cl)c1 ; N=C(NC(=N)Nc1cc(Cl)cc(Cl)c1)Nc1cc(Cl)cc(Cl)c1 ; CCCCNC(=N)N=C(N)N ; CCN(CC)C(=N)NC(=N)Nc1cccc(Cl)c1 ; N=C(N)/N=C(\N)NCCc1ccccc1]
and pharmaceutically acceptable salts thereof.

20. A method for establishing a prognosis for a patient having cancer, comprising: 1) obtaining a cancer cell sample from the patient; and 2) detecting whether expression of at least one CYP is increased and/or whether expression of soluble epoxide hydrolase (EPHX2) is decreased in a cancer cell from the sample, by measuring the expression level of the at least one CYP and/or EPHX2; and 3) establishing the prognosis is poor when increased expression of at least one CYP and/or decreased expression of EPHX2 is detected, as compared to a control.
